(19) United States
(12) Patent Application Publication
(10) Pub. No.: US 2024/0099271 A1
(43) Pub. Date: Mar. 28, 2024

(54) System And Method For Extracting Insights Through Analysis Of Behaviors Demonstrated By A Non-Human Animal

(71) Applicant: SUREFLAP LIMITED, Cambridge (GB)

(72) Inventors: Eran GENZEL, Netanya (IL); Pinhas SABO, Netanya (IL); Eden WEINBERG, Netanya (IL); Gal HAR ZION, Netanya (IL); Wibke METZ, Schwabenheim (DE); Michael Karl HINZ, Schwabenheim (DE); Inka Regine KUHLMANN, Schwabenheim (DE); Ulrich SONDERN, Schwabenheim (DE); Eva ZSCHIESCHE, Schwabenheim (DE); Alexander Patrick STEUDLE, Schwabenheim (DE); Brunhilde SCHÖLZKE, Schwabenheim (DE); Robert David ARMSTRONG, Madison, NJ (US); Robert Philip LAVAN, Rahway, NJ (US)

(21) Appl. No.: 18/066,294

(22) Filed: Dec. 15, 2022

Related U.S. Application Data
(60) Provisional application No. 63/376,664, filed on Sep. 22, 2022.

(51) Int. Cl. A01K 29/00; A01K 11/00; A01K 27/00

(52) U.S. Cl. CPC A01K 29/005; A01K 11/006; A01K 27/009

(57) ABSTRACT

A system for identifying irregularities in behaviors of a non-human animal, the system comprising a processing circuitry configured to: provide a behavioral baseline including first information on regular behaviors of the non-human animal over a given period of time when no irregularities occur; obtain data on a series of consecutively identified behaviors of the non-human animal identified over a second period of time; perform an action upon the data not complying with the behavioral baseline, thereby indicating an irregularity in the non-human animal behavior.

### TECHNICAL FIELD

The presently disclosed subject matter relates to extracting insights by analysis of behaviors demonstrated by non-human animals.

### BACKGROUND

An ongoing challenge for non-human animals' caregivers is the inability of non-human animals to communicate by explaining their feelings and/or emotions. Non-human animals cannot explain to their caregiver any details about their condition, or even point to a specific suffering for any reason, and caregivers are expected to identify when non-human animals suffer only by monitoring the non-human animals' behavior. On top of that, animals cannot be constantly monitored by their caregivers. Even if animals could communicate—in most cases it is not possible to extract insights specifically and objectively (such as one or more of: identify behavioral patterns, identify behavioral baselines, identify connections between behaviors, identify animals' conditions/health, or identify behavioral irregularities) from the animal's behavior in real time or even close to real time. Thus, for example, identification of an irregular behavior of any individual animal takes time as the caregiver is required to first observe the irregular behavior and then determine it is irregular. Even when noted, in many cases additional time will be allocated by the caregiver to ascertain that the irregular behavior is not temporary but a result of illness, infection, pruritus, or some other situation requiring intervention (examples of interventions are behavior modification, medical examination, or treatments). It can be easily appreciated that in some cases the longer it takes to identify a medical condition that requires intervention, the harder it is to treat the medical condition, and in extreme cases—the time window for treating the medical condition may close.

Looking at an example, a pet such as a dog or a cat can suffer from pruritus (itchiness) for various reasons associated with skin health (e.g. dermatologic diseases, allergies, etc.) and/or several different parasite infestations. In many cases, the pruritus may not be noted by the animal caregivers (e.g. the pet owner) as it may take time for the pet owner to recognize that their pet is exhibiting irregular or pathologic (rather than normal) behavior, such as increased or irregular scratching, shaking, barking, grooming, etc. In some cases, the pet owner may only be able to identify the pruritus after skin injury has already occurred which causes great discomfort to the animal Even when excessive scratching, shaking, barking, grooming, etc. is noted, the pet owner may decide to wait longer to make sure that the pruritus does not simply resolve without treatment. Thus, it may take considerable time until the pet owner takes the pet to the veterinarian for intervention (such as behavior modification, medical examination and/or treatment). The problem may have become much worse and even more difficult to resolve by the time treatment can be administered.

In some cases, a specific type of intervention may not solve the problem of the non-human animal. Again, due to the lack of ability to communicate with the non-human animals, and the inability of the animal caregiver to constantly monitor the animal, it may be difficult to determine the effectiveness of the intervention. Thus, an intervention may be ineffective, or only partially effective, and a long period of time may be required in order to understand that the intervention (such as behavior modification or treatment) does not solve the problem. In view of this fact, and by the time proper intervention is identified, the non-human animal may suffer for long periods of time until suitable intervention is identified and solves the problem. A related situation can occur when the caregiver does not or cannot comply with the care needs to resolve the problem. This lack of treatment adherence may not be apparent to the veterinarian unless the caregiver provides timely updates on the animal's status.

Another challenge is when a new type of intervention (such as a new type of behavior modification method or a new type of treatment) is developed and its efficacy is required to be determined. For example, when a new medication is developed, it is required to be tested for determining whether such new medication is effective, and potentially also how well it perform with respect to existing and potentially competing interventions (such as competing treatments).

One commonality to all of these situations is that measurement of the non-human animals' behaviors requires subjective observations such as documentation by the non-human animals' caregivers. Current technology enables determining animal behaviors by objective monitoring and analyzing of animal movement data (and/or other types of data, such as body position data) acquired over time, e.g. by a three-dimensional accelerometer that records specific movements made by the animal. One example is Sure Petcare's Animo® (hereinafter: “Animo”) Animo® is an activity and behavior monitor device that is attached to a dog, such as on a dog collar Animo® learns and accurately interprets the unique patterns of a dog Animo® delivers insights into a dog's activity and sleep patterns, and translates movements into named behaviors such as shaking, scratching and barking, which are potentially indicative of underlying differences from normal movement (wellbeing) Animo® measures movement of the dog in three dimensions (3D accelerometer). The dog's movement is recorded in time intervals, for each time interval, the movement data acquired during such time interval can be characterized into a named behavior. The characterized behaviors can include any one or more of the following, either alone or in combination: shaking, grooming, scratching, resting, sleeping, high-activity, medium activity, low-activity, barking, calories burned, walking, running, sitting, lying, jumping, chewing, sniffing, licking and more. Utilization of such technology can be useful in identifying valuable information (such as one or more of: behavioral patterns, behavioral baselines, connections between behaviors, animal's conditions/health, impact of treatment, or behavioral irregularities) by analyzing identified behaviors of non-human animals in an objective manner that does not depend on subjective human observation on the non-human animal's behaviors.

Animo® can be used for providing information that is useful for both research and development (e.g. for testing effects of medications, discovering new correlations between behaviors and animal's health status, etc.), and for consumers (providing pet owners with insights on their pet's wellbeing, improving communication between pet owners and veterinarians, etc.). It can be appreciated that the information can be provided in different manners for various purposes. For example, for research and development purposes, the data that can be obtained by using Animo® is substantially more granular than data that will be provided to consumers seeing the characterized named behaviors. Most consumers will not be able to understand the raw data collected by Animo® and therefore the data provided to consumers may be processed or analyzed to provide clearer insights to the consumers.

It is to be noted that devices that monitor movements of non-human animals and characterize behaviors are in use also with animals other than dogs, such as cattle. However, due to the inherent difference between different types of animals, the devices and the algorithms employed on the data collected thereby, are substantially different. Dogs have much more flexible movement than cattle for example (imagine a cow scratching in the ways that a dog can), and therefore the raw accelerometer data includes different types of physical movements than it is possible for cattle to make for example.

There is thus a need in the art for a new system and method for extracting insights (such as one or more of: identifying behavioral patterns, identifying behavioral baselines, identifying connections between behaviors, identifying animal's conditions/health, or identifying behavioral irregularities) through analysis of behaviors demonstrated by non-human animals.

### GENERAL DESCRIPTION

In accordance with a first aspect of the presently disclosed subject matter, there is provided a system for identifying irregularities in behaviors of a non-human animal, the system comprising a processing circuitry configured to: provide a behavioral baseline including first information on regular behaviors of the non-human animal over a given period of time when no irregularities occur; obtain data on a series of consecutively identified behaviors of the non-human animal identified over a second period of time; perform an action upon the data not complying with the behavioral baseline, thereby indicating an irregularity in the non-human animal behavior.

In one embodiment of the presently disclosed subject matter and/or embodiments thereof, the information on regular behaviors includes, for each regular behavior: (a) an indication of a type of behavior, and (b) one or more of: (i) regular frequency range of the behavior, (ii) regular duration range for the behavior, (iii) regular intensity range for the behavior, (iv) regular score range of a score calculated for the behavior.

In one embodiment of the presently disclosed subject matter and/or embodiments thereof, the processing circuitry is further configured to analyze the data to determine a cause for the irregularity.

In one embodiment of the presently disclosed subject matter and/or embodiments thereof, the cause is one or more of: pruritus, a cardiological problem, a neurological problem, obesity, diabetes, separation anxiety, arthritis, ear inflammation, a musculo-skeletal problems.

In one embodiment of the presently disclosed subject matter and/or embodiments thereof, the consecutively identified behaviors are determined based on analysis of three-dimensional (3D) accelerometer data acquired by a 3D accelerometer comprised in a device attached to the non-human animal.

In one embodiment of the presently disclosed subject matter and/or embodiments thereof, the behavioral baseline is an animal specific behavioral baseline determined using baseline creation data including a baseline series of consecutively identified baseline behaviors of the non-human animal identified over a third period of time in which the non-human animal is assumed to behave regularly.

In one embodiment of the presently disclosed subject matter and/or embodiments thereof, the action is triggering an alert to a caregiver of the non-human animal.

In one embodiment of the presently disclosed subject matter and/or embodiments thereof, the caregiver is an owner of the non-human animal, a veterinarian of the non-human animal, or a trainer of the non-human animal.

In one embodiment of the presently disclosed subject matter and/or embodiments thereof, the alert includes an indication of a potential cause for the irregularity.

In one embodiment of the presently disclosed subject matter and/or embodiments thereof, the regular behaviors and the consecutively identified behaviors include one or more of: shaking, grooming, scratching, resting, sleeping, high-activity, medium activity, low-activity, barking, calories burned, walking, running, sitting, lying, jumping, chewing, sniffing, or licking.

In one embodiment of the presently disclosed subject matter and/or embodiments thereof, the information on the regular behaviors includes a sleep score.

In one embodiment of the presently disclosed subject matter and/or embodiments thereof, the processing circuitry is further configured to provide one or more irregularity preventing recommendations to a caregiver of the non-human animal based on historical behavioral data associated with the non-human animal.

In accordance with a second aspect of the presently disclosed subject matter, there is provided a system for monitoring an effect of treatment of one or more causes for irregularities in behaviors of a non-human animal, the system comprising a processing circuitry configured to: provide a behavioral baseline including first information on regular behaviors of the non-human animal over a given period of time when no irregularities occur; obtain second information of a second series of consecutively identified behaviors of the non-human animal identified over a second period of time after application of the treatment to the non-human animal; perform an action upon a trend of one or more parameters calculated based on the second information not converging with the behavioral baseline.

In one embodiment of the presently disclosed subject matter and/or embodiments thereof, the first information on regular behaviors includes, for each regular behavior: (a) an indication of a type of behavior, and (b) one or more of: (i) regular frequency range of the behavior, (ii) regular duration range for the behavior, (iii) regular intensity range for the behavior, (iv) regular score range of a score calculated for the behavior.

In one embodiment of the presently disclosed subject matter and/or embodiments thereof, the consecutively identified behaviors are determined based on analysis of three-dimensional (3D) accelerometer data acquired by a 3D accelerometer comprised in a device attached to the non-human animal.

In one embodiment of the presently disclosed subject matter and/or embodiments thereof, the behavioral baseline is an animal specific behavioral baseline determined using baseline creation data including a baseline series of consecutively identified baseline behaviors of the non-human animal identified over a third period of time in which the non-human animal is assumed to behave regularly.

In one embodiment of the presently disclosed subject matter and/or embodiments thereof, the action is triggering an alert to a caregiver of the non-human animal.

In one embodiment of the presently disclosed subject matter and/or embodiments thereof, the caregiver is an owner of the non-human animal, a veterinarian of the non-human animal, or a trainer of the non-human animal.

In one embodiment of the presently disclosed subject matter and/or embodiments thereof, the regular behaviors and the consecutively identified behaviors include one or more of: shaking, grooming, scratching, resting, sleeping, high-activity, medium activity, low-activity, barking, calories burned, walking, running, sitting, lying, jumping, chewing, sniffing, or licking.

In one embodiment of the presently disclosed subject matter and/or embodiments thereof, the first information on the regular behaviors includes a sleep score.

In accordance with a third aspect of the presently disclosed subject matter, there is provided a system for monitoring an effect of treatment of one or more causes for irregularities in behaviors of a non-human animal, the system comprising a processing circuitry configured to: provide a successful treatment behavioral baseline including first information on regular behaviors of the non-human animal over a plurality of time periods following application of treatment of the one or more causes for irregularities in non-human animal behaviors; obtain second information of a series of consecutively identified behaviors of the non-human animal identified over a given time period after the application of the pruritus-causing infestations treatment to the non-human animal; perform an action upon the series of consecutively identified behaviors of the non-human animal not complying with the successful treatment behavioral baseline over a time period of the time periods, corresponding to the given time period.

In one embodiment of the presently disclosed subject matter and/or embodiments thereof, the first information on regular behaviors includes, for each regular behavior: (a) an indication of a type of behavior, and (b) one or more of: (i) regular frequency range of the behavior, (ii) regular duration range for the behavior, (iii) regular intensity range for the behavior, (iv) regular score range of a score calculated for the behavior.

In one embodiment of the presently disclosed subject matter and/or embodiments thereof, the consecutively identified behaviors are determined based on analysis of three-dimensional (3D) accelerometer data acquired by a 3D accelerometer comprised in a device attached to the non-human animal.

In one embodiment of the presently disclosed subject matter and/or embodiments thereof, the successful treatment behavioral baseline is an animal specific successful treatment behavioral baseline determined using baseline creation data including a baseline series of consecutively identified baseline behaviors of the non-human animal identified over a third period of time following application of the treatment of the one or more causes for the irregularities in the non-human animal behaviors.

In one embodiment of the presently disclosed subject matter and/or embodiments thereof, the action is triggering an alert to a caregiver of the non-human animal.

In one embodiment of the presently disclosed subject matter and/or embodiments thereof, the caregiver is an owner of the non-human animal, a veterinarian of the non-human animal, or a trainer of the non-human animal.

In one embodiment of the presently disclosed subject matter and/or embodiments thereof, the regular behaviors and the consecutively identified behaviors include one or more of: shaking, grooming, scratching, resting, sleeping, high-activity, medium activity, low-activity, barking, calories burned, walking, running, sitting, lying, jumping, chewing, sniffing, or licking.

In one embodiment of the presently disclosed subject matter and/or embodiments thereof, the first information on the regular behaviors includes a sleep score.

In accordance with a fourth aspect of the presently disclosed subject matter, there is provided a method for identifying irregularities in behaviors of a non-human animal, the method comprising: providing, by a processing circuitry, a behavioral baseline including first information on regular behaviors of the non-human animal over a given period of time when no irregularities occur; obtaining, by the processing circuitry, data on a series of consecutively identified behaviors of the non-human animal identified over a second period of time; performing, by the processing circuitry, an action upon the data not complying with the behavioral baseline, thereby indicating an irregularity in the non-human animal behavior.

In one embodiment of the presently disclosed subject matter and/or embodiments thereof, the information on regular behaviors includes, for each regular behavior: (a) an indication of a type of behavior, and (b) one or more of: (i) regular frequency range of the behavior, (ii) regular duration range for the behavior, (iii) regular intensity range for the behavior, (iv) regular score range of a score calculated for the behavior.

In one embodiment of the presently disclosed subject matter and/or embodiments thereof, the method further comprises analyzing, by the processing circuitry, the data to determine a cause for the irregularity.

In one embodiment of the presently disclosed subject matter and/or embodiments thereof, the cause is one or more of: pruritus, a cardiological problem, a neurological problem, obesity, diabetes, separation anxiety, arthritis, ear inflammation, a musculo-skeletal problems.

In one embodiment of the presently disclosed subject matter and/or embodiments thereof, the consecutively identified behaviors are determined based on analysis of three-dimensional (3D) accelerometer data acquired by a 3D accelerometer comprised in a device attached to the non-human animal.

In one embodiment of the presently disclosed subject matter and/or embodiments thereof, the behavioral baseline is an animal specific behavioral baseline determined using baseline creation data including a baseline series of consecutively identified baseline behaviors of the non-human animal identified over a third period of time in which the non-human animal is assumed to behave regularly.

In one embodiment of the presently disclosed subject matter and/or embodiments thereof, the action is triggering an alert to a caregiver of the non-human animal.

In one embodiment of the presently disclosed subject matter and/or embodiments thereof, the caregiver is an owner of the non-human animal, a veterinarian of the non-human animal, or a trainer of the non-human animal.

In one embodiment of the presently disclosed subject matter and/or embodiments thereof, the alert includes an indication of a potential cause for the irregularity.

In one embodiment of the presently disclosed subject matter and/or embodiments thereof, the regular behaviors and the consecutively identified behaviors include one or more of: shaking, grooming, scratching, resting, sleeping, high-activity, medium activity, low-activity, barking, calories burned, walking, running, sitting, lying, jumping, chewing, sniffing, or licking.

In one embodiment of the presently disclosed subject matter and/or embodiments thereof, the information on the regular behaviors includes a sleep score.

In one embodiment of the presently disclosed subject matter and/or embodiments thereof, the method further comprises providing, by the processing circuitry, one or more irregularity preventing recommendations to a caregiver of the non-human animal based on historical behavioral data associated with the non-human animal.

In accordance with a fifth aspect of the presently disclosed subject matter, there is provided a method for monitoring an effect of treatment of one or more causes for irregularities in behaviors of a non-human animal, the method comprising: providing, by a processing circuitry, a behavioral baseline including first information on regular behaviors of the non-human animal over a given period of time when no irregularities occur; obtaining, by the processing circuitry, second information of a second series of consecutively identified behaviors of the non-human animal identified over a second period of time after application of the treatment to the non-human animal; performing, by the processing circuitry, an action upon a trend of one or more parameters calculated based on the second information not converging with the behavioral baseline.

In one embodiment of the presently disclosed subject matter and/or embodiments thereof, the first information on regular behaviors includes, for each regular behavior: (a) an indication of a type of behavior, and (b) one or more of: (i) regular frequency range of the behavior, (ii) regular duration range for the behavior, (iii) regular intensity range for the behavior, (iv) regular score range of a score calculated for the behavior.

In one embodiment of the presently disclosed subject matter and/or embodiments thereof, the consecutively identified behaviors are determined based on analysis of three-dimensional (3D) accelerometer data acquired by a 3D accelerometer comprised in a device attached to the non-human animal.

In one embodiment of the presently disclosed subject matter and/or embodiments thereof, the behavioral baseline is an animal specific behavioral baseline determined using baseline creation data including a baseline series of consecutively identified baseline behaviors of the non-human animal identified over a third period of time in which the non-human animal is assumed to behave regularly.

In one embodiment of the presently disclosed subject matter and/or embodiments thereof, the action is triggering an alert to a caregiver of the non-human animal.

In one embodiment of the presently disclosed subject matter and/or embodiments thereof, the caregiver is an owner of the non-human animal, a veterinarian of the non-human animal, or a trainer of the non-human animal.

In one embodiment of the presently disclosed subject matter and/or embodiments thereof, the regular behaviors and the consecutively identified behaviors include one or more of: shaking, grooming, scratching, resting, sleeping, high-activity, medium activity, low-activity, barking, calories burned, walking, running, sitting, lying, jumping, chewing, sniffing, or licking.

In one embodiment of the presently disclosed subject matter and/or embodiments thereof, the first information on the regular behaviors includes a sleep score.

In accordance with a sixth aspect of the presently disclosed subject matter, there is provided a method for monitoring an effect of treatment of one or more causes for irregularities in behaviors of a non-human animal, the method comprising: providing, by a processing circuitry, a successful treatment behavioral baseline including first information on regular behaviors of the non-human animal over a plurality of time periods following application of treatment of the one or more causes for irregularities in non-human animal behaviors; obtaining, by the processing circuitry, second information of a series of consecutively identified behaviors of the non-human animal identified over a given time period after the application of the pruritus-causing infestations treatment to the non-human animal; performing, by the processing circuitry, an action upon the series of consecutively identified behaviors of the non-human animal not complying with the successful treatment behavioral baseline over a time period of the time periods, corresponding to the given time period.

In one embodiment of the presently disclosed subject matter and/or embodiments thereof, the first information on regular behaviors includes, for each regular behavior: (a) an indication of a type of behavior, and (b) one or more of: (i) regular frequency range of the behavior, (ii) regular duration range for the behavior, (iii) regular intensity range for the behavior, (iv) regular score range of a score calculated for the behavior.

In one embodiment of the presently disclosed subject matter and/or embodiments thereof, the consecutively identified behaviors are determined based on analysis of three-dimensional (3D) accelerometer data acquired by a 3D accelerometer comprised in a device attached to the non-human animal.

In one embodiment of the presently disclosed subject matter and/or embodiments thereof, the successful treatment behavioral baseline is an animal specific successful treatment behavioral baseline determined using baseline creation data including a baseline series of consecutively identified baseline behaviors of the non-human animal identified over a third period of time following application of the treatment of the one or more causes for the irregularities in the non-human animal behaviors.

In one embodiment of the presently disclosed subject matter and/or embodiments thereof, the action is triggering an alert to a caregiver of the non-human animal.

In one embodiment of the presently disclosed subject matter and/or embodiments thereof, the caregiver is an owner of the non-human animal, a veterinarian of the non-human animal, or a trainer of the non-human animal.

In one embodiment of the presently disclosed subject matter and/or embodiments thereof, the regular behaviors and the consecutively identified behaviors include one or more of: shaking, grooming, scratching, resting, sleeping, high-activity, medium activity, low-activity, barking, calories burned, walking, running, sitting, lying, jumping, chewing, sniffing, or licking.

In one embodiment of the presently disclosed subject matter and/or embodiments thereof, the first information on the regular behaviors includes a sleep score.

In accordance with a seventh aspect of the presently disclosed subject matter, there is provided a non-transitory computer readable storage medium having computer readable program code embodied therewith, the computer readable program code, executable by at least one processing circuitry of a computer to perform a method for identifying irregularities in behaviors of a non-human animal, the method comprising: providing, by the processing circuitry, a behavioral baseline including first information on regular behaviors of the non-human animal over a given period of time when no irregularities occur; obtaining, by the processing circuitry, data on a series of consecutively identified behaviors of the non-human animal identified over a second period of time; performing, by the processing circuitry, an action upon the data not complying with the behavioral baseline, thereby indicating an irregularity in the non-human animal behavior.

In accordance with a eighth aspect of the presently disclosed subject matter, there is provided a non-transitory computer readable storage medium having computer readable program code embodied therewith, the computer readable program code, executable by at least one processing circuitry of a computer to perform a method for monitoring an effect of treatment of one or more causes for irregularities in behaviors of a non-human animal, the method comprising: providing, by the processing circuitry, a behavioral baseline including first information on regular behaviors of the non-human animal over a given period of time when no irregularities occur; obtaining, by the processing circuitry, second information of a second series of consecutively identified behaviors of the non-human animal identified over a second period of time after application of the treatment to the non-human animal; performing, by the processing circuitry, an action upon a trend of one or more parameters calculated based on the second information not converging with the behavioral baseline.

In accordance with a nineth aspect of the presently disclosed subject matter, there is provided a non-transitory computer readable storage medium having computer readable program code embodied therewith, the computer readable program code, executable by at least one processing circuitry of a computer to perform a method for monitoring an effect of treatment of one or more causes for irregularities in behaviors of a non-human animal, the method comprising: providing, by the processing circuitry, a successful treatment behavioral baseline including first information on regular behaviors of the non-human animal over a plurality of time periods following application of treatment of the one or more causes for irregularities in non-human animal behaviors; obtaining, by the processing circuitry, second information of a series of consecutively identified behaviors of the non-human animal identified over a given time period after the application of the pruritus-causing infestations treatment to the non-human animal; performing, by the processing circuitry, an action upon the series of consecutively identified behaviors of the non-human animal not complying with the successful treatment behavioral baseline over a time period of the time periods, corresponding to the given time period.

### BRIEF DESCRIPTION OF THE DRAWINGS

In order to understand the presently disclosed subject matter and to see how it may be carried out in practice, the subject matter will now be described, by way of non-limiting examples only, with reference to the accompanying drawings, in which:

FIG. 1, a schematic illustration of an operational environment of a system for extracting insights from behaviors of non-human animals, in accordance with the presently disclosed subject matter;

FIG. 2 is a block diagram schematically illustrating one example of a system, in accordance with the presently disclosed subject matter;

FIG. 3 is a flowchart illustrating one example of a sequence of operations carried out for extracting insights from behaviors of non-human animals, in accordance with the presently disclosed subject matter;

FIG. 4 is a flowchart illustrating one example of a sequence of operations carried out for monitoring effects of intervention performed on non-human animals, in accordance with the presently disclosed subject matter;

FIG. 5 is another flowchart illustrating another example of a sequence of operations carried out for monitoring effects of intervention performed on non-human animals, in accordance with the presently disclosed subject matter;

FIGS 6a-9e show an exemplary Graphical User Interface (GUI) shown to dogs' caregivers, in accordance with the presently disclosed subject matter:

FIG. 6a shows a GUI indicating that a dog is suffering from excessive shaking;

FIG. 6b shows a GUI showing the shaking measurements of the dog of FIG. 7a throughout a period of one month, during which the dog was treated;

FIGS. 7a and 7b show a GUI indicating that a dog is suffering from excessive shaking and scratching prior to intervention and the effects of the intervention;

FIGS. 8a-8e show a GUI indicating that a dog is suffering from excessive shaking and reduced sleep quality prior to intervention and the effects of the intervention;

FIGS. 9a-9e show a GUI indicating that a dog is suffering from excessive shaking prior to intervention and the effects of the intervention;

FIGS. 10a-10g each shows a graph illustrating values of a parameter determined during a trial during which animals have been monitored in an infested period and a non infested period during the day and during night;

FIGS. 11a-11d each shows a graph illustrating the course of the mean number of events per hour between Day 8 and Day 24 of an exemplary study discussed herein;

FIG. 12 shows a graph illustrating the scratching minutes per month by dermatologic diagnosis as observed in an exemplary study discussed herein;

FIG. 13 shows a graph illustrating the average scratching minutes per day by dermatologic diagnosis as observed in an exemplary study discussed herein;

FIG. 14 shows a graph illustrating the average shaking minutes per day by dermatologic diagnosis as observed in an exemplary study discussed herein;

FIG. 15 shows a graph illustrating the average grooming minutes per day by dermatologic diagnosis as observed in an exemplary study discussed herein;

FIG. 16 shows a graph illustrating the average night rest minutes per night by dermatologic diagnosis as observed in an exemplary study discussed herein;

FIG. 17 shows a graph illustrating the average sleep ratio per night by dermatologic diagnosis as observed in an exemplary study discussed herein;

FIG. 18 shows a graph illustrating the average scratching minutes per day as observed in an exemplary study discussed herein;

FIG. 19 shows a graph illustrating the average shaking minutes per day as observed in an exemplary study discussed herein;

FIG. 20 shows a graph illustrating the average grooming minutes per day as observed in an exemplary study discussed herein; and

FIG. 21 shows a graph illustrating the average night rest minutes per night as observed in an exemplary study discussed herein.

### DETAILED DESCRIPTION

In the following detailed description, numerous specific details are set forth in order to provide a thorough understanding of the presently disclosed subject matter. However, it will be understood by those skilled in the art that the presently disclosed subject matter may be practiced without these specific details. In other instances, well-known methods, procedures, and components have not been described in detail so as not to obscure the presently disclosed subject matter.

In the drawings and descriptions set forth, identical reference numerals indicate those components that are common to different embodiments or configurations.

Unless specifically stated otherwise, as apparent from the following discussions, it is appreciated that throughout the specification discussions utilizing terms such as “providing”, “obtaining”, “performing”, “analyzing” or the like, include action and/or processes of a computer that manipulate and/or transform data into other data, said data represented as physical quantities, e.g. such as electronic quantities, and/or said data representing the physical objects. The terms “computer”, “processor”, “processing circuitry” and “controller” should be expansively construed to cover any kind of electronic device with data processing capabilities, including, by way of non-limiting example, a personal desktop/laptop computer, a server, a computing system, a communication device, a smartphone, a tablet computer, a smart television, a processor (e.g. digital signal processor (DSP), a microcontroller, a field programmable gate array (FPGA), an application specific integrated circuit (ASIC), etc.), a group of multiple physical machines sharing performance of various tasks, virtual servers co-residing on a single physical machine, any other electronic computing device, and/or any combination thereof.

The operations in accordance with the teachings herein may be performed by a computer specially constructed for the desired purposes or by a general-purpose computer specially configured for the desired purpose by a computer program stored in a non-transitory computer readable storage medium. The term “non-transitory” is used herein to exclude transitory, propagating signals, but to otherwise include any volatile or non-volatile computer memory technology suitable to the application.

As used herein, the phrase “for example,” “such as”, “for instance” and variants thereof describe non-limiting embodiments of the presently disclosed subject matter. Reference in the specification to “one case”, “some cases”, “other cases” or variants thereof means that a particular feature, structure or characteristic described in connection with the embodiment(s) is included in at least one embodiment of the presently disclosed subject matter. Thus, the appearance of the phrase “one case”, “some cases”, “other cases” or variants thereof does not necessarily refer to the same embodiment(s).

It is appreciated that, unless specifically stated otherwise, certain features of the presently disclosed subject matter, which are, for clarity, described in the context of separate embodiments, may also be provided in combination in a single embodiment. Conversely, various features of the presently disclosed subject matter, which are, for brevity, described in the context of a single embodiment, may also be provided separately or in any suitable sub-combination.

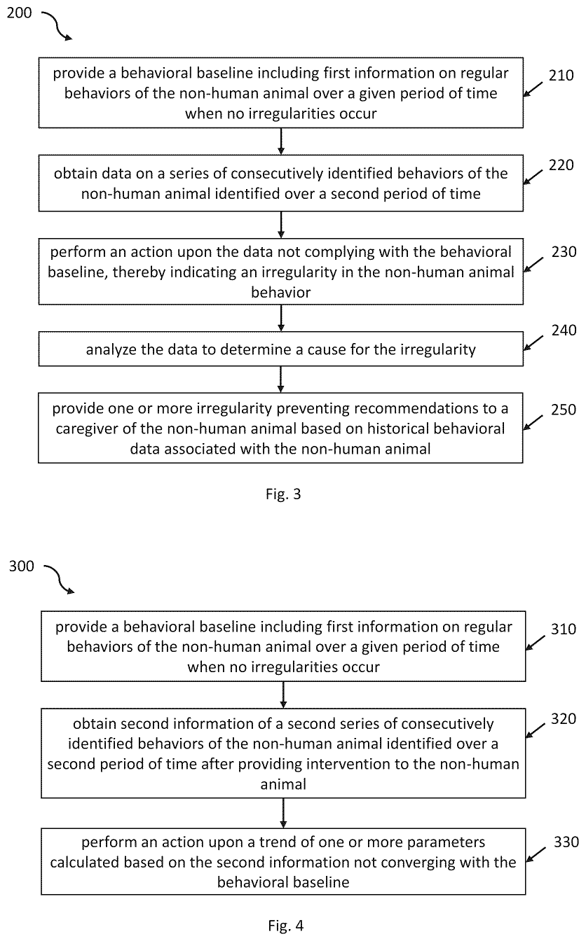

In embodiments of the presently disclosed subject matter, fewer, more and/or different stages than those shown in FIGS. 3-5 may be executed. In embodiments of the presently disclosed subject matter one or more stages illustrated in FIGS. 3-5 may be executed in a different order and/or one or more groups of stages may be executed simultaneously. FIG. 2 illustrates a general schematic of the system architecture in accordance with an embodiment of the presently disclosed subject matter. Each module in FIG. 2 can be made up of any combination of software, hardware and/or firmware that performs the functions as defined and explained herein. The modules in FIG. 2 may be centralized in one location or dispersed over more than one location, as detailed herein.

In other embodiments of the presently disclosed subject matter, the system may comprise fewer, more, and/or different modules than those shown in FIG. 2.

Any reference in the specification to a method should be applied mutatis mutandis to a system capable of executing the method and should be applied mutatis mutandis to a non-transitory computer readable medium that stores instructions that once executed by a computer result in the execution of the method.

Any reference in the specification to a system should be applied mutatis mutandis to a method that may be executed by the system and should be applied mutatis mutandis to a non-transitory computer readable medium that stores instructions that may be executed by the system.

Any reference in the specification to a non-transitory computer readable medium should be applied mutatis mutandis to a system capable of executing the instructions stored in the non-transitory computer readable medium and should be applied mutatis mutandis to method that may be executed by a computer that reads the instructions stored in the non-transitory computer readable medium.

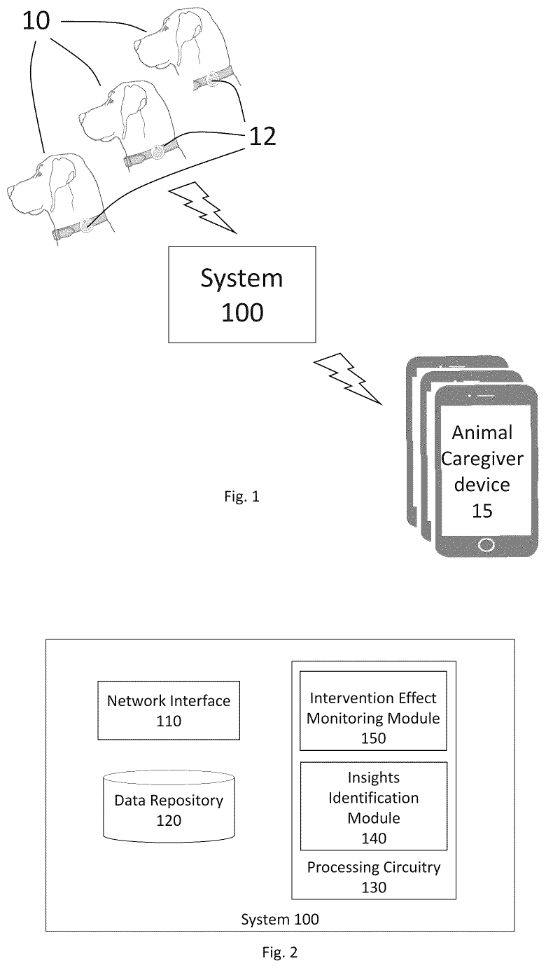

Bearing this in mind, attention is drawn to FIG. 1, a schematic illustration of an operational environment of a system for extracting insights from behaviors of non-human animals, in accordance with the presently disclosed subject matter.

In accordance with the presently disclosed subject matter, non-human animals 10 (such as pets, including dogs and cats, however not thus limited) are monitored using animal monitoring devices 12 such as Sure Petcare' s Animo® (hereinafter: “Animo”). The animal monitoring devices 12 can be attached, for example, to the animal's collar (as shown in the illustration), or in any other manner that enables the animal monitoring devices 12 to collect data enabling characterizing the behaviors of the non-human animal to which they are attached over time. It is to be noted that although in the illustration the animal monitoring devices 12 are attached to the non-human animals 10, in some cases, the animal monitoring devices 12 can be devices that do not require attachment to the non-human animal 10. One example is a monitoring device that utilizes an external camera that can monitor the animal's environment, in order to identify the animal and characterize its behaviors over time. Other examples of monitoring device that are not wearable include connected feeding and drinking stations, microphones (that can identify sounds made by the non-human animal 10 such as barking, yowling, etc.), weight scales, doorways, etc., each of which can be used in order to characterize behaviors of the non-human animals 10. It is to be noted that in some cases the behaviors can be determined using a combination of wearable and non-wearable devices.

As indicated herein, the animal monitoring devices 12 are configured to characterize the behaviors of the non-human animals 10 monitored thereby over time. Some exemplary behaviors that the animal monitoring devices 12 can identify include: shaking, grooming, scratching, resting, sleeping, high-activity, medium activity, low-activity, barking, calories burned, walking, running, sitting, lying, jumping, chewing, sniffing, licking, etc. and combinations thereof. However, it is to be noted that the animal monitoring devices 12 can be configured to identify only part of these behaviors, identify additional behaviors on top of the behaviors listed above, or combinations thereof.

In some cases, the animal monitoring device 12 can comprise a three-dimensional (3D) accelerometer, and in such cases, the behaviors can be identified by analyzing the data acquired by the 3D accelerometer, as each behavior has a corresponding manifestation in the acquired acceleration data.

Animal monitoring device 12 can optionally further include a location determination device (not shown), such as a Global Navigation Satellite System (GNSS) receiver, or any other device that can enable determining the geographical location of the animal monitoring device 12. Using the location determination device, the animal monitoring device 12 can track the location of the non-human animal 10 monitored thereby over time.

In some cases, the animal monitoring devices 12 can also measure (using various sensors), and optionally monitor, biometric and other information associated with the non-human animals 10 such as temperature, hormone levels, blood oxygen, electrocardiogram (ECG) data, food and/or water intake, urine production, blood pressure, other blood chemistries such as electrolytes, respiratory rate, heartbeat rate, Deoxyribonucleic acid (DNA) data, face recognition data, etc. It is to be noted that in some alternative or complementary cases, all, or part, of the biometric materials can be measured and monitored by sensors comprised within another device (not shown in the figure), other than the animal monitoring device 12. Such other device can also optionally be attached to the non-human animal 10 by various means (e.g. attached to the non-human animal's 10 collar).

In some cases, animal monitoring devices 12 are external to system 100 which is an independent entity which is communicatively connected to the animal monitoring devices. In such cases, the information related to the behaviors of each non-human animal 10, and optionally the location information determined using the locating determination device (referred to hereinafter as: “location information”), is transmitted to system 100. However, in some cases system 100 can be part of the animal monitoring devices 12 so that each, or some, of the animal monitoring devices 12 is a standalone unit that is capable of performing the operations of system 100, as detailed herein.

In those cases where the animal monitoring devices 12 are external to system 100, the transmission can be direct from the animal monitoring devices 12 to system 100 (e.g. when the animal monitoring devices 12 have Internet connectivity). Alternatively, the information of the behaviors of each non-human animal 10, and optionally the location information, can be transmitted from the animal monitoring devices 12 to system 100 indirectly. In these cases, the information of the behaviors of each non-human animal 10, and optionally the location information, can be transmitted from the animal monitoring devices 12 to an intermediary device (e.g. any device that has Internet connectivity, including, for example, a smartphone) from which the information is transmitted to system 100. The information of the behaviors of each non-human animal 10, and optionally the location information, can be transmitted from the animal monitoring devices 12 to the intermediary device via a short-range connection such as a Bluetooth Low Energy (BLE) connection. This configuration supports lower energy consumption of the animal monitoring device 12, which in turn enable longer recharging cycles thereof.

System 100 is configured to extract insights (such as one or more of: identifying behavioral patterns, identifying behavioral baselines, identifying connections between behaviors, identifying animal's conditions/health, or identifying behavioral irregularities) from the behaviors of the non-human animals 10 by analyzing the behaviors of the non-human animals 10 as determined by the animal monitoring devices 12, optionally along with the location information, and as further detailed herein, inter alia with reference to FIG. 3. Once an insight requires an action (such as when an irregularity is identified in the non-human animal's 10 behavior), system 100 can be configured to perform an action, such as triggering an alert to a caregiver (such as its owner and/or its veterinarian and/or its trainer) of the non-human animal 10 in question. The alert can be provided to the caregiver via an animal caregiver device 15, such as a smartphone, a personal computer, a laptop computer, a smartwatch, or any other type of device through which the alert can be provided to the non-human animal's caregiver.

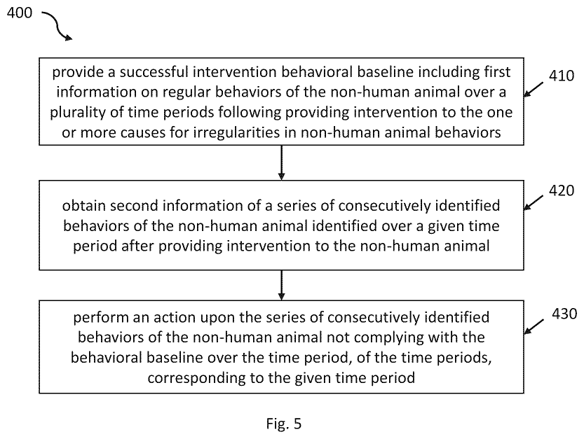

Additionally, or alternatively, system 100 can be configured to monitor an effect of intervention (such as behavior modification or treatment), e.g. in response to a health condition (noting that a health condition can be a physical health condition such as pruritus, etc., or a mental health condition, such as anxiety) or irregular behavior of the non-human animal 10, as further detailed herein, inter alia with reference to FIGS. 4-5. For example, when an irregularity is identified (e.g. based on analysis of the behaviors of a non-human animal 10), in many cases the irregularity has a cause. The cause for an irregularity can be, for example, pruritus, a cardiological problem, a neurological problem, obesity, diabetes, separation anxiety, arthritis, a musculo-skeletal problem, etc. When the cause for the irregularity is identified, in many cases the non-human animal 10 receives treatment, such as medicine, antiparasitic products (e.g. a Fluralaner formulation such as Merck Animal Health's Bravecto®), or other types of intervention. Such intervention (such as behavior modification or treatment) should gradually, or in some cases immediately, stop the irregularity in the non-human animal's 10 behaviors. System 100 can analyze the behaviors of the non-human animal 10 after the intervention, and verify that the non-human animal's 10 behaviors after the intervention (e.g. after treatment is provided or at least prescribed), are gradually decreasing, or immediately stopping, the irregularities

In some cases, upon the intervention not demonstrating an expected effect (e.g. gradually decreasing, or immediately stopping, the irregularities), system 100 can perform an action, such as triggering an alert to a caregiver of the non-human animal 102 (such as its owner and/or its veterinarian and/or its trainer) whose intervention does not yield the expected, or desired, results. The alert can be provided to the caregiver via an animal caregiver device 15, such as a smartphone, a personal computer, a laptop computer, a smartwatch, or any other type of device through which the alert can be provided to the non-human animal's 10 caregiver.

In some cases, system 100 can enable communication between the non-human animal's 10 owner and other entities such as the non-human animal's 10 veterinarian or veterinary clinic staff. For example, the non-human animal's 10 owner can add notes via a suitable device (e.g. a smartphone, a personal computer, a laptop computer, a smartwatch, or any other type of device on which an application is installed which supports uploading such notes). Such notes can be sent to the non-human animal's 10 veterinarian via a suitable device (e.g. a smartphone, a personal computer, a laptop computer, a smartwatch, or any other type of device on which an application is installed which supports receiving such notes). Additionally, or alternatively, the non-human animal's 10 veterinarian can send information to the non-human animal's 10 owner, such as notes, prescriptions, reminders, etc. In a more general sense, system 100 can enable communication between various animal caregiver devices 15 (e.g. animal caregiver devices 15 of veterinarians or veterinarians clinic staff of the non-human animals 10, and animal caregiver devices 15 of the non-human animals 10 owners).

System 100 can additionally, or alternatively, generate reports, and various recommendations, such as training or diet recommendations. The system 100 can optionally utilize additional devices for these purposes, including, for example, non-human animal's 10 bowl/water consumption information obtained from suitable non-human animal's 10 bowls that can monitor consumption of food and/or water. System 100 can optionally also help non-human animal's 10 trainer in learning the historical behavior of the non-human animal 10 and optionally creating a recommendation/training program for the non-human animal 10. In some cases, system 100 can also track the results and outcomes of the training program and optionally adjust it accordingly.

Having described the operational environment of the system 10, attention is drawn to FIG. 2, a block diagram schematically illustrating one example of a system for extracting insights from behaviors of non-human animals, in accordance with the presently disclosed subject matter.

According to the presently disclosed subject matter, system 100 comprises a processing circuitry 130. Processing circuitry 130 can be one or more processing units (e.g. central processing units), microprocessors, microcontrollers (e.g. microcontroller units (MCUs)) or any other computing devices or modules, including multiple and/or parallel and/or distributed processing units, which are adapted to independently or cooperatively process data for controlling relevant system 100 resources and for enabling operations related to system's 100 resources.

Processing circuitry 100 can comprise an insights identification module 140 and/or an intervention effect monitoring module 150. Insights identification module 140 is configured to extract insights from non-human animal's (such as pets, including dogs, cats, etc.) behaviors, as further detailed herein, inter alia with reference to FIG. 2. Intervention effect monitoring module 150 is configured to monitor effects of intervention on non-human animal's behaviors, as further detailed herein, inter alia with reference to FIGS. 3 and 4.

System 100 can further comprise a network interface 110 (e.g. a network card, a WiFi client, a LiFi client, 3G/4G client, or any other component), enabling system 100 to communicate over a network with various systems, such as external systems that can provide system 100 with behavioral data characterizing behaviors of one or more non-human animals over time, and optionally with location data indicative of the location of the non-human animals over time. One example of such external system is animal monitoring devices 12 such as Sure Petcare's Animo®. It is to be noted that in some cases the animal monitoring devices 12 (such as Sure Petcare's Animo®) may provide the behavioral data, and optionally the location data, to system 100 directly (via a network connection such as WiFi or cellular communication) or indirectly via user devices (such as smartphones, laptops, smart speakers, smartwatches, etc.) to which the animal monitoring devices 12 can connect via a relatively short-range connection such as Bluetooth Low Energy (BLE). On the other hand, in other cases, system 100 can be independent of any external systems, as it can be incorporated into an animal monitoring device 12 such as Sure Petcare's Animo® (and in such cases it may not require a network interface, or it may suffice having a relatively short-range connection such as Bluetooth Low Energy (BLE)).

System 100 can further comprise, or be otherwise associated with, a data repository 120 (e.g. a database, a storage system, a memory including Read Only Memory—ROM, Random Access Memory—RAM, or any other type of memory, etc.) configured to store data, optionally including, inter alia, for each non-human animal 10 being monitored: the non-human animal's 10 breed, the non-human animal's 10 age, the non-human animal's 10 gender, the non-human animal's 10 medical information (past diseases, medications received, etc.), special notes related to the non-human animal's 10, dislike, the non-human animal's 10 name, reports generated for the non-human animal's 10, information of a monitoring device attached to the non-human animal's 10 (such as the device's hardware/firmware/software version, etc.), information of past behaviors of the non-human animal 10 (such as shaking, grooming, scratching, resting, sleeping, high-activity, medium activity, low-activity, barking, calories burned, walking, running, sitting, lying, jumping, chewing, sniffing, licking, etc.), a behavioral baseline defining expected regular behaviors of the non-human animal 10, information of caregivers of the non-human animal 10 (e.g. the animal's owner, the animal's veterinarian, the animal's trainer, etc.), information of medical conditions of the non-human animal 10 (allergies, cardiac problems, neurological problems, diabetes, obesity, a musculo-skeletal problem, etc.), a successful intervention behavioral baseline defining expected changes in behaviors of the non-human animal 10 over time after providing intervention (such as behavior modification or treatment) when behavioral irregularities (e.g. an irregular pattern of behaviors, an irregular combination of behaviors, etc.) are identified, location information indicative of locations of the non-human animal 10 over time, etc. Data repository 120 can be further configured to enable retrieval and/or update and/or deletion of the stored data. It is to be noted that in some cases, data repository 120 can be distributed, while the system 100 has access to the information stored thereon, e.g. via a wired or wireless network to which system 100 is able to connect (e.g. via its network interface 110).

Attention is now drawn to FIG. 3, a flowchart illustrating one example of a sequence of operations carried out for extracting insights from behaviors of non-human animals, in accordance with the presently disclosed subject matter.

According to certain examples of the presently disclosed subject matter, system 100 can be configured to perform an insights identification process 200, e.g. utilizing the insights identification module 140.

For this purpose, system 100 can be configured to provide a behavioral baseline including information on regular behaviors of a non-human animal 10 over a given period of time when no irregularities occur (block 210).

As indicated herein, the animal monitoring devices 12 are configured to characterize the behaviors of the non-human animals 10 monitored thereby over time. For example, 3D accelerometer data can be continuously acquired, and analyzed in time windows (e.g. every three/five/ten seconds) in order to determine a behavior of the animal during the analyzed time period. Some exemplary behaviors that the animal monitoring devices 12 can identify include: shaking, grooming, scratching, resting, sleeping, high-activity, medium activity, low-activity, barking, calories burned, walking, running, sitting, lying, jumping, chewing, sniffing, licking, etc. This information can be used in order to create a behavioral baseline, e.g. as further detailed herein (while noting that the behavioral baseline can alternatively be determined in other manners, or it can be received from another system).

It is to be noted that in some cases the behavioral baseline can be associated with a subset of one or more behaviors out of the available behaviors (available to system 100), or with a combination of some, or all, of the available behaviors. Thus, the baseline can be provided for a single behavior (e.g. a sleeping baseline (sleeping can be defined, for example, by a sleep score), a scratching baseline, a shaking baseline, etc.), or for a combination of behaviors (e.g. a sleeping and shaking baseline, a sleeping and scratching baseline, etc.). It is to be noted that in such cases, the behavioral baseline will be built based on information associated with such subset of behaviors when no irregularities that may have an effect on those behaviors occur (while other irregularities that do not have an effect on those behaviors can occur as they do not have an impact on the baseline which is based on behaviors that are not effected by such irregularities).

It is to be further noted that the baseline can vary by time of day, season, types of activity the animal is engaged in (sleeping, barking, running), different periods of time covered by the baseline (e.g. 1 hour, 12 hours, 24 hours, one week, one month, one year, etc.). In some cases, multiple baselines can exist and be used according to time of day (e.g. a daytime baseline and a nighttime baseline), season (a summer baseline, a winter baseline, a spring baseline and an autumn baseline), types of activity the animal is engaged in (a barking baseline, a sleeping baseline, a running baseline, etc.), etc.

The behavioral baseline can be an animal specific behavioral baseline determined using baseline creation data including a baseline series of consecutively identified baseline behaviors of the non-human animal identified over a period of time in which the non-human animal is assumed to behave regularly (i.e. it is not sick, not suffering from pruritus, etc.). Put in other words, the behavioral baseline can be determined by system 100 using the information of the non-human animals' 10 behaviors over a given period of time as determined using the information acquired by the animal monitoring devices 12. As indicated herein, it is to be noted that the behavioral baseline can be determined in other manners (for example, the behavioral baseline can be determined based on human observations on the animal), or a combination of data from animal monitoring devices 12 and human observation can be used to obtain, and or refine, a baseline.

It is to be noted that in some cases the behavioral baseline can be a general baseline that is determined for a group of non-human animals 10 and not for a specific animal The group can be of non-human animals of the same breed/type/size/etc.

Furthermore, in some cases system 100 can use a general baseline for a newly monitored non-human animal 10, and such baseline can be improved when specific behavioral data is collected by the animal monitoring device 12 attached to the newly monitored non-human animal 10. In a more general sense, it is to be noted that in some cases, the behavioral baseline can be updated or refined over time as more data is collected by the animal monitoring device/s 12. It is to be noted that this may be required as non-human animal's behavioral patterns may change between seasons, as they get older, due to injuries, etc.

It is to be noted that in some cases various parameters can have an effect on an expected behavioral baseline, such as the location (city/urban/rural/village) of the non-human animal 10, age of the non-human animal 10, age of the non-human animal's 10 owner, etc. In such cases, a plurality of general baseline (that are not animal specific) can exist, and a specific non-human animal 10 can be associated with a selected baseline based on the relevant parameters.

The information on regular behaviors which is included in the behavioral baseline can include, for each regular behavior: (a) an indication of a type of behavior (e g a name of the behavior, a graph indicative of the type of behavior, a digital signal characterizing the type of behavior, or any other data that enables differentiating between the behavior and other behaviors), and (b) one or more of: (i) regular frequency range of the behavior (e.g. how many times can one expect to see the behavior during a given time period), (ii) regular duration range for the behavior (e.g. how long can one expect the behavior to last), (iii) regular intensity range for the behavior (e.g. how intense can one expect the behavior to be), (iv) regular score range of a score calculated for the behavior (e.g. a score range of a sleep score calculated for the non-human animal 10), (v) regular time windows during which the behavior is expected to occur, etc.

Looking at a specific example, a behavioral baseline can define that a certain non-human animal 10 is expected to sleep between 8-11 hours, rest between 4-6 hours, groom between 15-45 minutes, shake between 10-20 minutes, scratch between 20-40 minutes, be in high activity between 30-60 minutes, be in medium activity between 45-90 minutes, be in low activity between 1-3 hours, eat between 10-30 minutes, all during a time period of 24 hours. As a further example, the behavioral baseline can also define a regular grooming and scratching intensity range, frequency, duration.

For example, the behavioral baseline can also define a regular duration for each occurrence of one or more of the behaviors, such as scratching. Accordingly, the baseline can define that during a time period of 24 hours scratching occurs between 20-40 minutes, however each single scratching behavior that occur during the 24 hours' time period occurs between 5-45 seconds consecutively.

The behavioral baseline can also define that a range of a sleep score calculated for the sleeping behavior is between 75-85 out of 100. It is to be noted that the sleep score can be determined based on a measurement of the duration and frequency of behaviors that can be associated with interruption of sleep and on a measurement of the duration of uninterrupted sleep (so that, in general, the longer the duration of uninterrupted sleep is—the higher the sleep score is). It is to be noted in this respect that the sleep score enables highlighting irregularities as the non-human animal's 10 behaviors are expected to change less frequently than during the daytime when the non-human animal 10 is more active, assuming that the non-human animal 10 does not have a medical condition that requires intervention. In case the non-human animal 10 does not have a medical condition that requires intervention, it is expected to be less active during the sleeping times and it's behavior is expected to be quiet or less interrupted (in the sense that it is not expected to have many interferences caused by grooming or shaking), and if during these times the non-human animal 10 is more active than usual (e.g. shaking and/or grooming more than usual), it can enable identification of the medical condition that requires intervention.

System 100 is further configured to obtain data on a series of consecutively identified behaviors of the non-human animal 10 identified over a second period of time, other than the one based on which the behavioral baseline was determined (if so determined) (block 220). The consecutively identified behaviors can be determined based on analysis of three-dimensional (3D) accelerometer data acquired by a 3D accelerometer comprised in an animal monitoring device 12 attached to the non-human animal 10.

The series of consecutively identified behaviors can be analyzed in order to determine if the consecutively identified behaviors of the series comply with the behavioral baseline, and in case they do not comply with the behavioral baseline, system 100 is configured to perform an action, thereby providing an insight associated with the non-human animal's behavior (e.g. indicating an irregularity in the non-human animal 10 behavior) (block 230). The action can be triggering an alert to a caregiver of the non-human animal 10, such as the non-human animal's 10 owner and/or a veterinarian of the non-human animal 10 and/or a trainer of the non-human animal 10.

In some cases, the alert can be provided to the non-human animal's 10 owner recommending a visit of the veterinarian. In some cases, the owner or caregiver of the non-human animal can contact the veterinarian based upon the owner's review of the alerts from the system. Using the system 100, the veterinarian can look at the objectively monitored data instead of the (subjective) description of the non-human animal's 10 owner only, which can be very helpful for the veterinarian.

It is to be noted, in this respect, that currently veterinarians do not have the ability to identify irregularities in behaviors of non-human animal 10, aside from being notified of it by the non-human animal's 10 owner, e.g. through traditional written or verbal sources (or in those cases where the non-human animal 10 can be observed by the veterinarian). Having the ability to be provided with information of irregularities in behaviors of non-human animal 10 that is treated by the veterinarian (noting that when reference is made herein to a veterinarian, it is not necessarily limited to a specific veterinarian, and it can be any veterinarian from a clinic in which the non-human animal 10 is treated) is extremely important, and it can result in saving non-human animal 10 lives, preventing (or at least reducing) undue suffering of the non-human animal 10, etc. This is emphasized in view of the fact that irregularities in behaviors of non-human animal 10 can be identified even before they are noticed by the non-human animal 10 owner. It should also be noted that providing the veterinary clinic and/or the veterinarian/s with such valuable information that can facilitate proactivity by the veterinary clinic and/or the veterinarians (e.g. by inviting the non-human animal 10 owner for a checkup of the non-human animal 10, etc.), provides the veterinary clinic and/or the veterinarians with a perceived added value by their clients (the non-human animals 10 owners).

In addition, and although not shown in the figure, a veterinary clinic in which one or more veterinarians are providing treatment to a plurality of non-human animals 10, can find great value in having the ability to obtain detailed reports about the behaviors of the non-human animal/s 10 treated in the veterinary clinic. Having such reports can enable better monitoring of the medical condition of the non-human animal/s 10, it can enable the clinic to proactively identify irregularities which require intervention, etc. The reports can be provided for parts of the non-human animal/s 10, e.g. for non-human animal/s 10 treated by a specific veterinarian, for non-human animal/s 10 the meet a certain condition (such as an age, sex, location, etc.), for non-human animal/s 10 that demonstrate certain behavior or combination of behaviors, etc. In some cases, the reports can be generated based on data acquired during a selected time period (e.g. during the last 24 hours, during the last week, during the last month, etc.), and in some cases they can show trends in various behavior/s over such time period. It can be appreciated that system 100 can be configured to generate any of the above-mentioned reports, or any other report, based on data acquired by the animal monitoring device 12 attached to the non-human animal 10 (including location data) and/or data acquired by other devices (such as non-human animal's 10 bowls that can monitor consumption of food and/or water, tracking devices that monitor an animal's location, implants that monitor a animal's biometric data, animal data from a database or similar, etc.).

It is to be noted that such reports may be provided through communication linkages between the system's 100 output and the veterinary practice management software and hardware. This can link the output to the medical record of the non-human animal 10 at the veterinary practice and facilitate communication with the veterinary practice personnel.

It is to be further noted that some reports can enable various comparisons between different non-human animal 10 populations, such as between a population of “normal” non-human animals 10 (that are not diagnosed as having any sickness) and an population of non-human animals 10 that are suffering from itches due to various reasons, or between a population of non-human animals 10 that have a specific disease and a population of non-human animals 10 that are suffering from itches due to various reasons.

Returning to, and continuing the example provided herein, in order to determine compliance of the series of consecutively identified behaviors of the non-human animal 10 with the behavioral baseline, the time spent by the given animal behaving in each type of behavior during the second period of time (as indicated by the series of consecutively 20 identified behaviors obtained at block 220) can be aggregated and compared with the corresponding expected range. If the time spent by the animal behaving in each type of behavior is not within the expected range according to the behavioral baseline, an irregularity is identified. Similarly, for each behavior that is associated with an expected intensity range by the behavioral baseline, any deviation from the expected intensity range can be identified as an irregularity. Furthermore, when the baseline defines a sleep score range, the actual sleep score calculated for the non-human animal 10 can be compared thereto, and upon the sleep score calculated for the non-human animal 10 deviating from the sleep score range an irregularity is identified.

It is to be noted that using the insights identification process 200 enables reducing the time required in order to identify insights, including early detection of health conditions/irregularities in the non-human animal's 10 behavior, as in many cases non-human animal's 10 caregiver is unable to note the behavioral changes until the behavioral changes are much more prominent than those that can be identified as insights (e.g. irregularities) by system 100.

In a specific study, staff of a veterinary clinic chose to call dog owners after multiple days observing unusual scratch or shake alerts (provided to the staff via system 100). This enables the staff to arrive at insights that may have been missed without this basic information being provided to them via system 100.

In some cases, system 100 is further configured to analyze the data obtained at block 220 to determine a cause for the insight (e.g. a cause for an identified irregularity) (block 240). Some exemplary causes for an irregularity that was identified as an insight can include one or more of: dermatological problems such as allergies/atopic dermatitis, otitis, parasite infestations (scratching, grooming, shaking, reduced sleeping score), a, obesity (objective control of activity and feeding), diabetes (reduced sleeping score), separation anxiety (increased barking), arthritis and other problems of the musculo-sceletal system (changes in activity pattern), or any pathological changes to any physiological activity affecting the non-human animal 10.

It can be appreciated that different causes for an irregularity will be manifested differently in the non-human animal's 10 behavioral data. For example, pruritus will cause a certain type of irregularity (such as lower than expected sleep score, excessive scratching, increased nighttime grooming, some combination, etc.), whereas arthritis will cause another type of irregularity (such as lower than expected high activity). The type and nature of the identified pruritus or arthritis may further help the veterinarian to assess potential underlying causes of the pathological condition(s).

In those cases where a factor or potential underlying reason contributing to the insight (e.g. a cause for an identified irregularity) is determined by system 100, an alert provided to the non-human animal's 10 caregiver can include an indication of the cause for the insight, which can be used by the caregiver in order to monitor the non-human animal 10, treat the non-human animal 10, consult with a veterinarian, etc.

In some cases, system 100 can utilize the location information acquired by the animal monitoring device 12 (and more specifically by the location determination device) in order to provide evidence for the underlying cause for the insight. For example, if the non-human animal 10 was located at a known flea-risk area, the cause for the insight can be identified as becoming flea infested. As another example, if the owner of the non-human animal 10 did not take it out for a walk, or changed its walking routine, the cause for the irregularity can be identified as missing a walk or changing the walking routine. It is to be noted that these are mere examples and the location can be used to determine the cause for the insight in other manners as well.

It is to be further noted that in some cases, if the location information can be used to explain an insight, system 100 can be configured not to perform the action at block 230, as there may be no need to provide an alert to a caregiver of the non-human animal 10. For example, if the non-human animal 10 had a longer walk than usual, which caused an irregularity in the non-human animal's 10 behavior (e.g. excessive resting during a certain time period after such a walk)—there may be no need to alert the caregiver of the irregularity, as it can be explained by the longer than usual walk.

In some cases, system 100 can be further configured to provide one or more irregularity preventing recommendations to a caregiver of the non-human animal 10 based on historical behavioral data associated with the non-human animal 10 (block 250). Accordingly, if analysis of past behavioral data of the non-human animal 10 indicates that at certain times of the year (e.g. during spring), the non-human animal 10 has allergies, system 100 can provide the caregiver with an irregularity preventing recommendation to start preventative intervention (such as behavior modification or treatment) in order to prevent allergies at the appropriate time (e.g. the beginning of the spring). It is to be noted that in some cases block 250 can be performed by itself, irrespective of the insights identification process 200.

In some cases, the historical behavioral data associated with the non-human animal 10 can be used in order to adapt the behavioral baseline to past changes in the non-human animal's 10 behaviors over time. For example, if the non-human animal 10 sleeps more during the winter, the baseline can be adjusted to reflect the fact that the non-human animal 10 sleeps more during winter than during summer, for example.

It is to be still further noted that, with reference to FIG. 3, some of the blocks can be integrated into a consolidated block or can be broken down to a few blocks and/or other blocks may be added. Furthermore, in some cases, the blocks can be performed in a different order than described herein. It is to be further noted that some of the blocks are optional. It should be also noted that whilst the flow diagram is described also with reference to the system elements that realizes them, this is by no means binding, and the blocks can be performed by elements other than those described herein.

Turning to FIG. 4, there is shown a flowchart illustrating one example of a sequence of operations carried out for monitoring effects of intervention (such as behavior modification or treatment) on non-human animals, in accordance with the presently disclosed subject matter.

According to certain examples of the presently disclosed subject matter, system 100 can be configured to perform an intervention (such as behavior modification or treatment including medical treatment and/or changed diet, and/or more exercise, etc.) effect monitoring process 300, e.g. utilizing the intervention effect monitoring module 150.

For this purpose, system 100 can be configured to provide a behavioral baseline including information on regular behaviors of a non-human animal 10 over a given period of time when no irregularities occur in the non-human animal's 10 behavior (block 310), similarly to the behavioral baseline discussed with reference to block 210.

System 100 is further configured to obtain information of a series of consecutively identified behaviors of the non-human animal 10 identified over a second period of time after providing intervention to the non-human animal 10 (block 320), similarly to the information of a series of consecutively identified behaviors of the non-human animal 10 discussed with reference to block 220, except that the series of consecutively identified behaviors of the non-human animal 10 in block 320 is identified over a time period after providing intervention to the non-human animal 10.

As can be appreciated, when a certain non-human animal 10 is provided with intervention, it is expected to have a positive effect on the irregularities in the behaviors of the non-human animal's 10. Accordingly, system 100 can be configured to perform an action upon a trend of one or more parameters calculated based on the information obtained at block 320 not converging with the behavioral baseline provided at block 310 (block 330). The action can be triggering an alert to a caregiver of the non-human animal 10, such as the non-human animal's 10 owner and/or a veterinarian of the non-human animal 10, and/or a trainer of the non-human animal 10). Such alert can enable adjusting the intervention or providing an additional or an alternative intervention (e.g. an alternative treatment) to the non-human animal 10, if required.

It is to be noted that the behavioral baseline can be animal specific, or it can be a generic behavioral baseline that can optionally be determined using experts estimates. In some cases, various generic baselines can exist, each of which can be associated with a set of parameters, such as specific breed/s, specific age range/s, specific animal size/s, specific medical diagnoses, etc. In such cases, a specific non-human animal 10 can be associated with a selected generic baseline, selected out of the collection of available baselines, based on a matching between the specific non-human animal 10 parameters and the parameters of the baselines in the collection.

It is to be noted that in some cases an animal specific baseline may not be available (e.g. for an animal that just started using Animo) In such cases, the intervention effect monitoring process 300 can still be performed, using a generic behavioral baseline, or even without using a baseline, but simply by making sure there is a positive trend that can be observed from the information obtained at block 320.

It is to be still further noted that, with reference to FIG. 4, some of the blocks can be integrated into a consolidated block or can be broken down to a few blocks and/or other blocks may be added. It should be also noted that whilst the flow diagram is described also with reference to the system elements that realizes them, this is by no means binding, and the blocks can be performed by elements other than those described herein.

FIG. 5 is another flowchart illustrating another example of a sequence of operations carried out for monitoring effects of intervention on non-human animals, in accordance with the presently disclosed subject matter.

According to certain examples of the presently disclosed subject matter, system 100 can be configured to perform another intervention effect monitoring process 400, e.g. utilizing the intervention effect monitoring module 150. The veterinarian or animal health provider would determine the specific details of the intervention and then the implementation and outcome of this intervention can be measured objectively by system 100.

For this purpose, system 100 can be configured to provide a successful intervention behavioral baseline including information on regular behaviors of the non-human animal 10 over a plurality of time periods following providing intervention (such as behavior modification or treatment including medical treatment and/or changed diet, and/or more exercise, etc.) to deal with the one or more causes for irregularities in non-human animal 10 behaviors (block 410). It is to be noted in this respect that successful intervention is expected to result in a progressive improvement in the non-human animal's condition over time, until full recovery and return to normalcy. Accordingly, the successful intervention behavioral baseline can define an expected improvement pattern of behavioral changes that the non-human animal is expected to demonstrate when a successful intervention is provided thereto.

It is to be noted that in some cases, a plurality of successful intervention behavioral baselines can exist, each associated with a specific cause for irregularities in the non-human anima's 10 behavior. For example, a first successful intervention behavioral baseline can be associated with pruritus, and a second successful intervention behavioral baseline can be associated with arthritis.

It is to be noted that different animals may respond differently to the same treatment. Thus, the successful intervention behavioral baseline can be an animal specific successful intervention behavioral baseline determined using baseline creation data including a baseline series of consecutively identified baseline behaviors of the non-human animal 10 identified over the plurality of time periods following providing intervention (such as behavior modification or treatment) to deal with the one or more causes for irregularities in non-human animal 10. Put in other words, the successful intervention behavioral baseline can be determined by system 100 using the information of the non-human animals' 10 behaviors over each time period of the plurality of time periods as determined using the information acquired by the animal monitoring devices 12. As indicated herein, it is to be noted that the successful intervention behavioral baseline can be determined in other manners (for example, the successful intervention behavioral baseline can be determined based on human observations on the non-human animal 10, or a combination of human observations and monitoring data).

It is to be noted that in some cases the successful intervention behavioral baseline can be a general baseline that is determined for a group of non-human animals 10 and not for a specific animal The group can be of non-human animals of the same breed/type/size/etc.

Furthermore, in some cases system 100 can use a general successful intervention behavioral baseline for a newly monitored non-human animal 10, and such baseline can be improved when specific behavioral data is collected by the animal monitoring device 12 attached to the newly monitored non-human animal 10 after providing intervention (such as behavior modification or treatment) to deal with the one or more causes for irregularities in non-human animal 10 behaviors, if the intervention was successful (as the successful treatment behavioral baseline is required to reflect improvement in behaviors when a successful intervention is provided).

The information on regular behaviors which is included in the successful intervention behavioral baseline can include, for each regular behavior at each time period: (a) an indication of a type of behavior (e g a name of the behavior, a description of the behavior, a graph indicative of the type of behavior, a digital signal characterizing the type of behavior, or any other data that enables differentiating between the behavior and other behaviors), and (b) one or more of: (i) regular frequency range of the behavior during the respective time period (e.g. how many times can one expect to see the behavior during the respective time period after intervention), (ii) regular duration range for the behavior during the respective time period (e.g. how long can one expect the behavior to last during the respective time period), (iii) regular intensity range for the behavior during the respective time period (e.g. how intense can one expect the behavior to be during the respective time period), (iv) regular score range of a score calculated for the behavior for the respective time period (e.g. a score range of a calculated sleep score calculated for the non-human animal 10 for the respective time period).

Looking at a specific example, a successful intervention behavioral baseline can define that a certain non-human animal 10, in the first 24 hours after the intervention, is expected to sleep between 6-9 hours, rest between 3-4 hours, groom between 1-2 hours, shake between 1-2 hours, scratch between 1-2 hours, be in high activity between 20-40 minutes, be in medium activity between 1-2 hours, be in low activity between 2-4 hours, eat between 10-20 minutes, all during the first 24 hours after the intervention. The successful intervention behavioral baseline can also define a regular grooming and scratching intensity range for those 24 hours.

The successful intervention behavioral baseline can also define a regular duration for each occurrence of one or more of the behaviors, such as scratching. Accordingly, the successful intervention behavioral baseline can define that during a time period of 24 hours after the intervention scratching occurs between 1-2 hours, however each single scratching behavior that occur during the 24 hours' time period occurs between 10-90 seconds consecutively.

The successful intervention behavioral baseline can also define that a range of a sleep score calculated for the sleeping behavior during the first 24 hours after the intervention, such as, between 60-75 out of 100. It is to be noted that the sleep score can be determined based on a measurement of the duration and frequency of behaviors that can be associated with interruption of sleep and on a measurement of the duration of uninterrupted sleep (so that the longer the duration of uninterrupted sleep is—the higher the sleep score is).

Continuing the example, the successful intervention behavioral baseline can define that a certain non-human animal 10, in the next 24 hours after the intervention (i.e. 24 hours to 48 hours after the intervention), is expected to exhibit improvement in its behaviors compared to the first 24 hours after the intervention. It can be expected to sleep between 8-11 hours, rest between 4-6 hours, groom between 15-45 minutes, shake between 10-20 minutes, scratch between 20-40 minutes, be in high activity between 30-60 minutes, be in medium activity between 45-90 minutes, be in low activity between 1-3 hours, eat between 10-30 minutes, all during the first 24 hours after the intervention. The successful intervention behavioral baseline can also define a regular grooming and scratching intensity range for those next 24 hours, which can be expected to be lower than the regular grooming and scratching intensity range for the first 24 hours. Additionally, the successful intervention behavioral baseline can define that during those next 24 hours (i.e. 24 hours to 48 hours after the intervention) scratching occurs between 0.5-1.5 hours, however each single scratching behavior that occur during this time period occurs between 5-60 seconds consecutively. The successful intervention behavioral baseline can also define that the range of a sleep score calculated for the sleeping behavior during the next 24 hours (i.e. 24 hours to 48 hours after the intervention), is expected to be between 70-85 out of 100.

It is to be noted that although in the example above only two time periods are provided for the successful intervention behavioral baseline, this is by no means limiting and the successful intervention behavioral baseline can include more than two time periods.

System 100 is further configured to obtain information of a series of consecutively identified behaviors of the non-human animal identified over a given time period after the pruritus-causing resolution-related intervention (such as behavior modification or treatment) being provided to the non-human animal (block 420). The consecutively identified behaviors can be determined based on analysis of three-dimensional (3D) accelerometer data acquired by a 3D accelerometer comprised in an animal monitoring device 12 attached to the non-human animal 10.

The series of consecutively identified behaviors can be analyzed in order to determine if the series of consecutively identified behaviors of the non-human animal does not comply with the successful intervention behavioral baseline over the time period, of the time periods, corresponding to the given time period, and in case they do not comply with the successful intervention behavioral baseline, system 100 is configured to perform an action, thereby indicating that the intervention is not performing as expected (block 430). The action can be triggering an alert to a caregiver of the non-human animal 10, such as the non-human animal's 10 owner and/or a veterinarian of the non-human animal 10, and/or a trainer of the non-human animal 10.

Continuing the example provided herein, in order to determine compliance of the series of consecutively identified behaviors of the non-human animal 10 with the successful intervention behavioral baseline, the time spent by the given animal behaving in each type of behavior during each of the plurality of periods of time (as indicated by the series of consecutively identified behaviors obtained at block 220) can be aggregated and compared with the corresponding expected range for the respective time period. If the time spent by the animal in each related behavioral classification is not within the expected range at the respective period of time, then the intervention is not performing as expected. Similarly, for each behavior that is associated with an expected intensity range by the successful intervention behavioral baseline, any deviation from the expected intensity range at the respective period of time can be indicative of the fact that the intervention is not performing as expected. Furthermore, when the successful intervention behavioral baseline defines a sleep score range for each time period, the actual sleep score calculated for the non-human animal 10 for the respective period can be compared thereto, and upon the sleep score calculated for the non-human animal 10 deviating from the sleep score range an insight is identified indicating an irregularity and potential pathologic situation.

The successful intervention behavioral baseline can be animal specific, or it can be a generic successful intervention behavioral baseline that can optionally be determined using experts estimates on expected successful intervention results. In some cases, various generic successful intervention behavioral baselines can exist, each of which can be associated with a set of parameters, such as specific breed/s, specific age range/s, specific animal size/s, specific medical diagnoses, etc. In such cases, a specific non-human animal 10 can be associated with a selected generic successful intervention behavioral baseline, selected out of the collection of available successful intervention behavioral baselines, based on a matching between the specific non-human animal 10 parameters and the parameters of the successful intervention behavioral baselines in the collection.

It is to be noted that in some cases an animal specific baseline may not be available (e.g. for an animal that just started using Animo) In such cases, the intervention effect monitoring process 400 can still be performed, using a generic successful intervention behavioral baseline, or even without using a baseline, but simply by making sure there is a positive trend that can be observed from the information obtained at block 420.

It is to be noted that using the intervention effect monitoring process 300 and/or the another intervention effect monitoring process 400 enables reducing the time required following treatment initiation in order to identify that intervention provided to the non-human animal 10 behavior is not performing as expected, as in many cases non-human animal's 10 caregiver is unable to note the behavioral changes that are expected to occur when the intervention is successful until the behavioral changes are much more prominent than those that can be identified as irregularities by system 100.

It is to be noted that when reference is made herein to any type of baseline, the baseline can be a dynamic baseline that can be calculated based on a certain period (e.g. a certain number of days/weeks/months/etc.), that can optionally be a rolling time window, during which the non-human animal's 10 behaved regularly.

It is to be still further noted that, with reference to FIG. 5, some of the blocks can be integrated into a consolidated block or can be broken down to a few blocks and/or other blocks may be added. It should be also noted that whilst the flow diagram is described also with reference to the system elements that realizes them, this is by no means binding, and the blocks can be performed by elements other than those described herein.

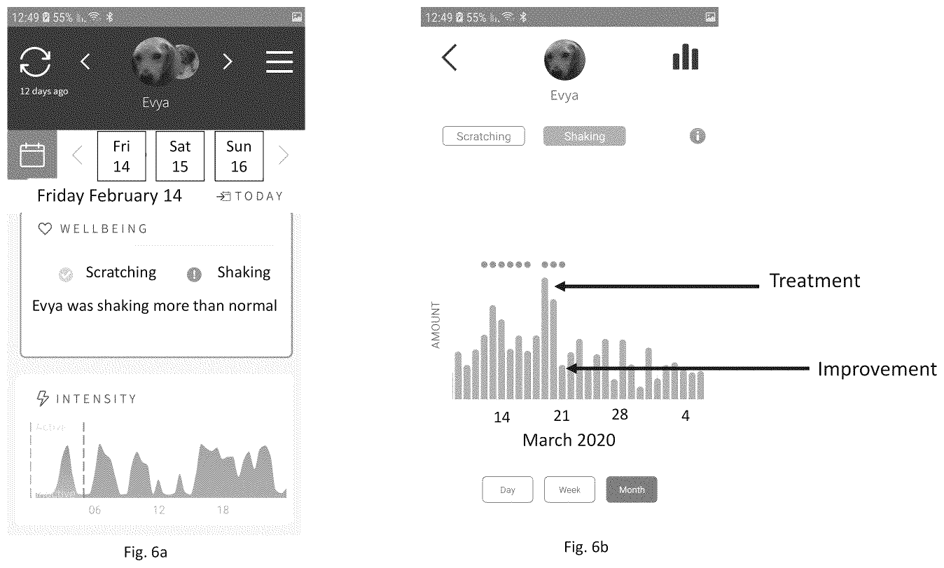

FIGS. 6-9 show an exemplary Graphical User Interface (GUI) shown to dogs' caregivers, in accordance with the presently disclosed subject matter. FIG. 6a shows a GUI indicating that a dog named “Evya” is suffering from excessive shaking (based on information acquired by an Animo® device attached to Evya's collar). Evya is a 16-year-old mixed dog, suffering from recurring otitis externa (ear inflammation). FIG. 6b shows a GUI showing the shaking measurements of Evya (based on information acquired by an Animo® device attached to Evya' s collar) throughout a period of one month, during which Evya was treated. As can be seen, Evya started getting an intervention (medical treatment) on Mar. 19, 2020, and by Mar. 21, 2020, a substantial improvement was recorded indicating that the shaking amount returned to a normal level for Evya at that period of time. Prior to the improvement, for a period of 9 days (marked by dots above the measured amount in the figure) Evya demonstrated excessive shaking, indicating that she was bothered by the ear inflammation. This example demonstrates, among other things, that the systems and methods disclosed herein can be used both to alert a caregiver to activity that might warrant an intervention and also track the intervention to see if it is successful.

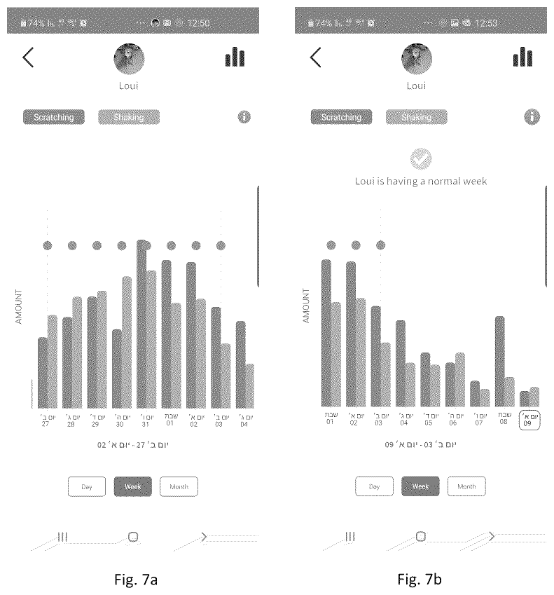

FIGS. 7a and 7b show a GUI indicating that a dog is suffering from excessive shaking and scratching prior to intervention, and the effects of the intervention. The information presented is associated with a dog named “Loui”, a 5-year-old beagle. Loui was brought in for surgery due to lip fold dermatitis on Jan. 26, 2020. The operation went well and Loui was dismissed wearing an Elizabethan Collar (cone shaped). Following the operation and due to the presence of the Elizabethan collar, Loui shook and scratched intensely for 8 days (as shown in FIGS. 7a and 7b showing information acquired by an Animo® device attached to Loui's collar). With the Elizabethan collar Loui could not bite her paws and it caused her much discomfort, as can be seen in FIGS. 7a and 7b, in which the dots indicate days in which Loui scratched more than usual. After the operation (Jan. 26, 2020), Loui didn't sleep well, and spent a lot of time scratching and shaking (but not grooming—as the Elizabethan collar prevents grooming). On Jul. 2, 2020, the Elizabethan collar was removed and Loui demonstrated excessive grooming (compensatory grooming). Loui was treated with steroids and antihistamine to prevent intense scratching and grooming, and on Jul. 4, 2020 Loui's shaking and scratching behaviors (based on information acquired by an Animo® device attached to Loui's collar) returned to normal levels, as can be seen in FIG. 7b.

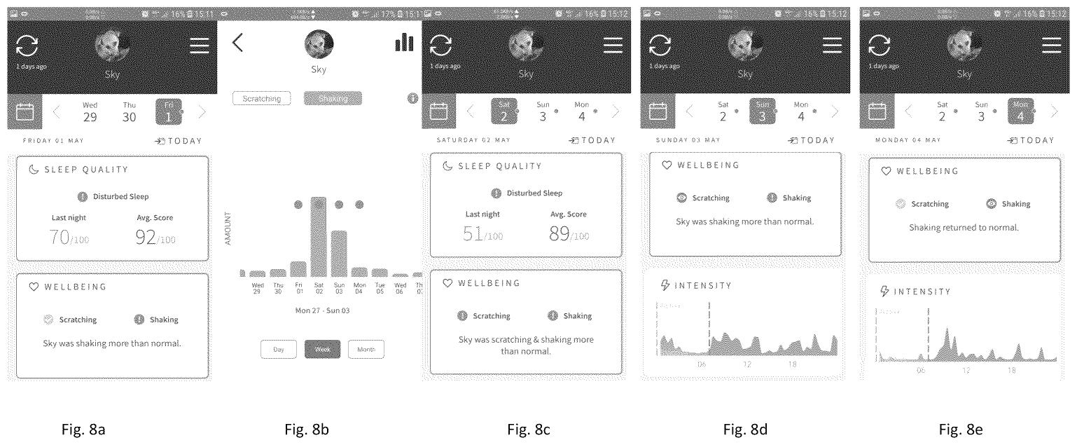

FIGS. 8a-8e show a GUI indicating that a dog is suffering from excessive shaking and reduced sleep quality prior to intervention and the effects of the intervention. The information presented is associated with a dog named “Sky”, a female mixed dog. Sky had an ear inflammation starting on May 1, 2020, and received intervention (medical treatment) for the infection on May 4, 2020. As can be seen in FIGS. 8a, on May 1, 2020, Sky's sleep score (based on information acquired by an Animo® device attached to sky's collar) dropped to 70 out of 100, compared to an average sleep score of 92 out of 100. In addition, as can be seen on FIG. 8b, Sky also started shaking more than usual on the same day (based on information acquired by an Animo® device attached to sky's collar). As can be seen in FIG. 8c, on the next day (May 2, 2020), Sky's sleep score further decreased to 51 out of 100, and it also demonstrated excessive shaking and scratching (based on information acquired by an Animo® device attached to sky's collar). As can be seen in FIG. 8d, on the next day (May 3, 2020), Sky's scratching values returned to the normal range, whereas Sky was still shaking more than usual. As can be seen in FIG. 8e, On May 4, 2020, Sky's behaviors (based on information acquired by an Animo® device attached to sky's collar) returned to normalcy.

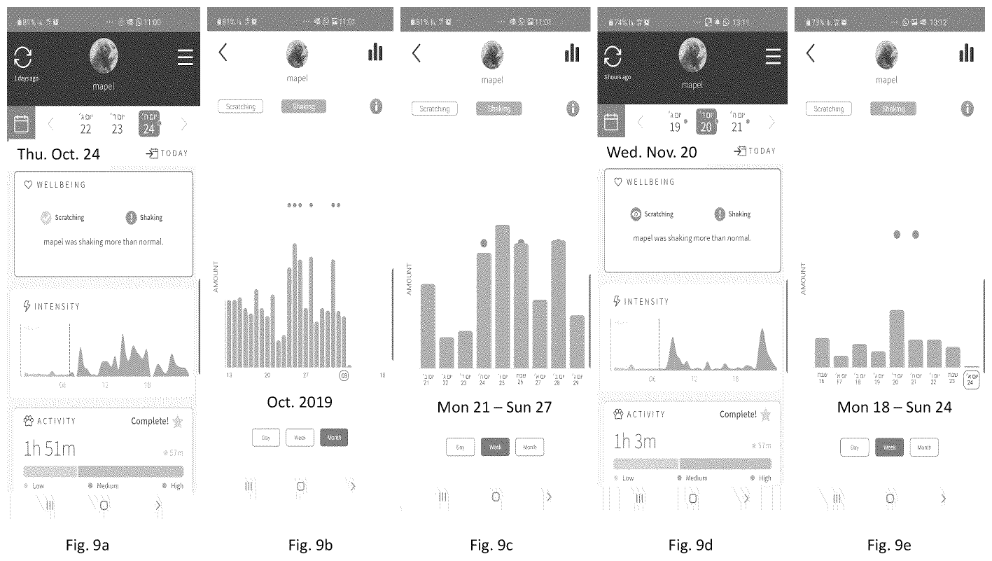

FIGS. 9a-9e show a GUI indicating that a dog is suffering from excessive shaking prior to intervention and the effects of the intervention. The information presented is associated with a dog named “Maple”, a 4-year-old cocker spaniel suffering from recurring Otitis Externa (Ear inflammation). On Oct. 31, 2019 Maple's owner complained of suspicion of ear inflammation in a visit to the veterinarian. As can be seen in FIG. 9a, on Oct. 24, 2019 Maple started shaking more than normal (based on information acquired by an Animo® device attached to Maple's collar). FIGS. 9b and 9c show that the excessive shaking took place also on Oct. 25, 26 and 28, 2019, prior to the visit to the veterinarian (based on information acquired by an Animo® device attached to Maple's collar). After the visit and beginning of intervention (medical treatment) for ear inflammation, the shaking decreased on Nov. 2, 2019 and returned to normal levels.

As can be seen in FIGS. 9d and 9e, ear inflammation reappeared which caused Maple to excessively shake on Nov. 20, 2019 and on Nov. 21, 2019. on Nov. 21, 2019 Maple was prescribed a different treatment which quickly reduced the excessive shaking, so that on Nov. 22, 2019, no excessive shaking was identified, indicating that the intervention was successful.

Before continuing with the figures, attention is drawn to another exemplary case study made in accordance with the teachings herein. The case study was made using a three-dimensional accelerometer, cloud data recording, and data presenting app (Animo® app, however not thus limited) to assist with medical management and early flare-up detection related to chronic dermatologic disease in a dog. Medical management of chronic canine pruritic dermatologic conditions is challenging and often frustrating. Getting early warning of flare ups, obtaining dog owner adherence to recommended treatment protocols, and maintaining close patient condition monitoring are factors that can dramatically improve the outcome. Movement monitoring using an accelerometer with cloud data recording, data analysis, and data presentation on a smartphone-based app (Animo® app, however not thus limited), optionally combined with automatic real time communication with the veterinary practice, can help to address these challenges and provide an opportunity for improved medical management.

In this case study, a male neutered 9-year-old 6 kg Pug cross dog, participating in a larger clinical study evaluating accelerometer technology with the owner's informed consent, was under medical management of skin disease. The dog was previously diagnosed with a chronic pruritic condition with previous flare ups and was referred to a veterinary dermatologist. The dog's pruritus was suspected to be atopic dermatitis associated with hypersensitivity to environmental allergens. The dog was also known to have previous episodes of otitis externa that resolved with treatment. Analyzed accelerometer data provided warning of a flare up in his pruritic condition before noticeable clinical signs were observed by the dog's owner. Based on this alert, communication was initiated between the veterinarian and dog owner that led to the modification of the pruritus management protocol and improvement in clinical signs.

To conclude, analyzed accelerometer data combined with data communication are valuable adjuncts for ongoing medical management of chronic pruritic skin disease of dogs.

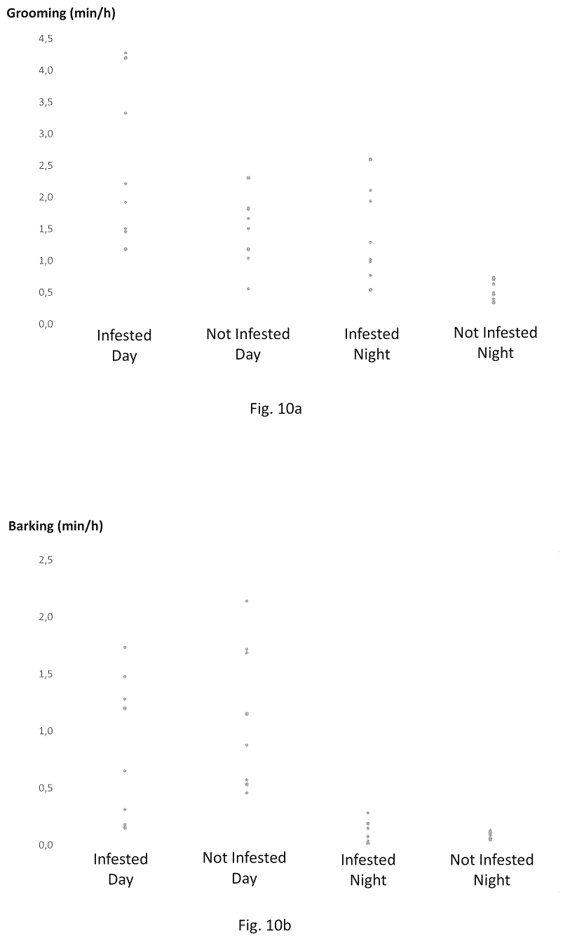

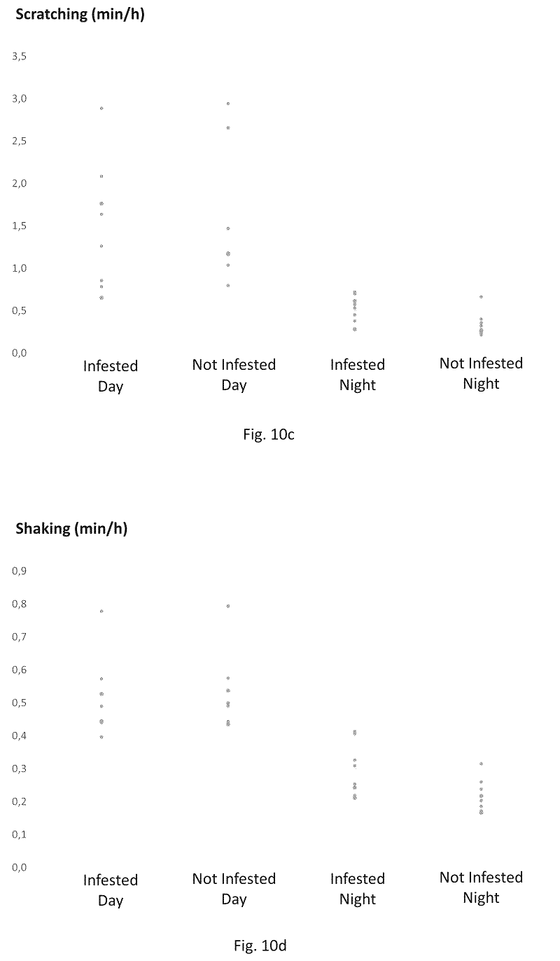

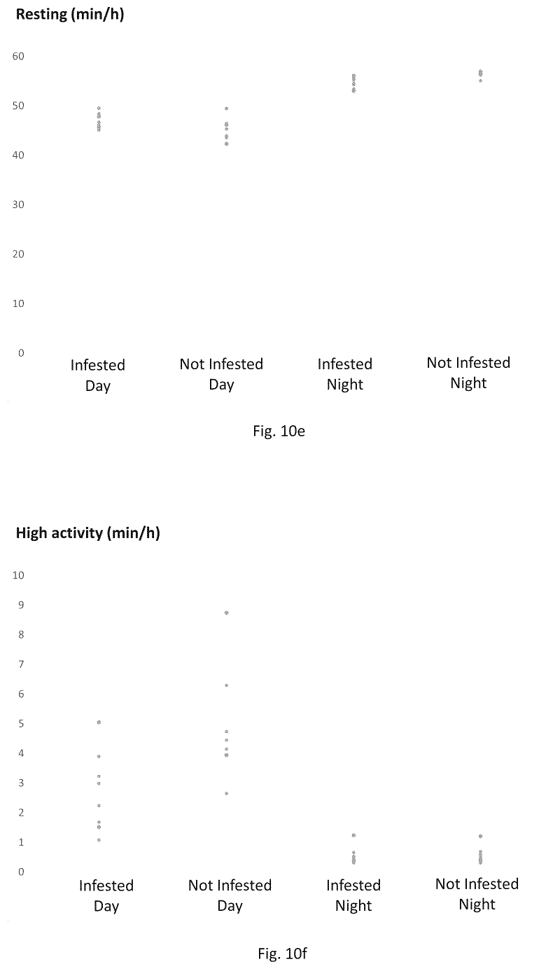

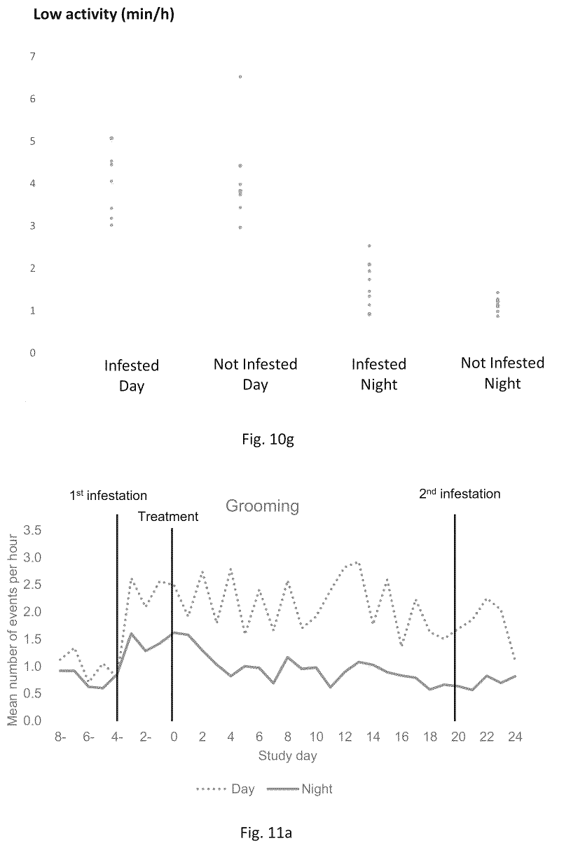

Returning to the figures, FIGS. 10a-10g each shows a graph illustrating values of a parameter determined during a trial during which animals have been monitored in a flea infested period and a flea non infested period during the day and during night. A more detailed explanation about the trial is provided hereinbelow. The information displayed in each figure is based on analysis of behaviors of eight dogs in an infested period, in which they are infested with Ctenocephalides felis (C. felis) fleas, and in a not-infested period in which they are not infested with Ctenocephalides felis (C. felis) fleas.

FIG. 10a shows an amount of grooming (minutes per hour) during: (a) daytime when the dogs are not infested with Ctenocephalides felis (C. felis) fleas, (b) nighttime when the dogs are not infested with Ctenocephalides felis (C. felis) fleas, (c) daytime when the dogs are infested with Ctenocephalides felis (C. felis) fleas, and (d) nighttime when the dogs are infested with Ctenocephalides felis (C. felis) fleas. Looking at the information, it can be appreciated that when infested with Ctenocephalides felis (C. felis) fleas, the dogs are involved in Grooming for longer times, both at daytime and nighttime, compared to the amount of grooming when the dogs are not infested with Ctenocephalides felis (C. felis) fleas. It can also be appreciated that the differences are much more significant during nighttime. At nighttime, when the dogs are not infested, they groom less than 1 minute every hour. When the dogs are infested, most dogs groom substantially more.

FIG. 10b shows an amount of barking (minutes per hour) during: (a) daytime when the dogs are not infested with Ctenocephalides felis (C. felis) fleas, (b) nighttime when the dogs are not infested with Ctenocephalides felis (C. felis) fleas, (c) daytime when the dogs are infested with Ctenocephalides felis (C. felis) fleas, and (d) nighttime when the dogs are infested with Ctenocephalides felis (C. felis) fleas. Looking at the information, it can be appreciated that when infested with Ctenocephalides felis (C. felis) fleas, the dogs are barking less during daytime and barking more during nighttime compared to the amount of barking when the dogs are not infested with Ctenocephalides felis (C. felis) fleas.

FIG. 10c shows an amount of scratching (minutes per hour) during: (a) daytime when the dogs are not infested with Ctenocephalides felis (C. felis) fleas, (b) nighttime when the dogs are not infested with Ctenocephalides felis (C. felis) fleas, (c) daytime when the dogs are infested with Ctenocephalides felis (C. felis) fleas, and (d) nighttime when the dogs are infested with Ctenocephalides felis (C. felis) fleas. Accordingly, it can be seen that scratching at night is lower in non-flea infested dogs.

FIG. 10d shows an amount of shaking (minutes per hour) during: (a) daytime when the dogs are not infested with Ctenocephalides felis (C. felis) fleas, (b) nighttime when the dogs are not infested with Ctenocephalides felis (C. felis) fleas, (c) daytime when the dogs are infested with Ctenocephalides felis (C. felis) fleas, and (d) nighttime when the dogs are infested with Ctenocephalides felis (C. felis) fleas. Looking at the information, it can be appreciated that when infested with Ctenocephalides felis (C. felis) fleas, the dogs are involved in shaking for longer times at nighttime, compared to the amount of shaking when the dogs are not infested with Ctenocephalides felis (C. felis) fleas during nighttime.

FIG. 10e shows an amount of resting (minutes per hour) during: (a) daytime when the dogs are not infested with Ctenocephalides felis (C. felis) fleas, (b) nighttime when the dogs are not infested with Ctenocephalides felis (C. felis) fleas, (c) daytime when the dogs are infested with Ctenocephalides felis (C. felis) fleas, and (d) nighttime when the dogs are infested with Ctenocephalides felis (C. felis) fleas. Accordingly, it can be seen that resting at night is higher in non-flea infested dogs.

FIG. 10f shows an amount of high activity (minutes per hour) during: (a) daytime when the dogs are not infested with Ctenocephalides felis (C. felis) fleas, (b) nighttime when the dogs are not infested with Ctenocephalides felis (C. felis) fleas, (c) daytime when the dogs are infested with Ctenocephalides felis (C. felis) fleas, and (d) nighttime when the dogs are infested with Ctenocephalides felis (C. felis) fleas. Looking at the information, it can be appreciated that when infested with Ctenocephalides felis (C. felis) fleas, the dogs are less involved in high activity during daytime, compared to the amount of high activity when the dogs are not infested with Ctenocephalides felis (C. felis) fleas during daytime.

FIG. 10g shows an amount of low activity (minutes per hour) during: (a) daytime when the dogs are not infested with Ctenocephalides felis (C. felis) fleas, (b) nighttime when the dogs are not infested with Ctenocephalides felis (C. felis) fleas, (c) daytime when the dogs are infested with Ctenocephalides felis (C. felis) fleas, and (d) nighttime when the dogs are infested with Ctenocephalides felis (C. felis) fleas. Looking at the information, it can be appreciated that when infested with Ctenocephalides felis (C. felis) fleas, the dogs are more involved in low activity during nighttime, compared to the amount of low activity when the dogs are not infested with Ctenocephalides felis (C. felis) fleas during nighttime. This reduced rest could further contribute to the reduced high activity time in flea-infested dogs in the daytime (refer to FIG. 10f).

Having described the figures, attention is now drawn to some studies that have been conducted in relation with the presently disclosed subject matter. It is to be noted that these are mere examples and should not be used to limit the scope of other parts of the detailed description.

### Example Study 1

### List of Abbreviations

| | |
|---|---|
| am | At morning |
| BW | Body weight |
| CAS | Chemical Abstracts Service |
| D | Study day |
| n | Number |
| NA | Not applicable |
| pm | Past midday |
| C. felis | Ctenocephalides felis |

### SUMMARY

This study evaluated the suitability of the Animo® device (Animo®) for monitoring the wellbeing of dogs by using a model (experimental infestation with Ctenocephalides felis (C. felis) fleas).

Materials and Methods: A total of 8 healthy dogs carrying the Animo® device were included in the study. Following 17 days of acclimatisation to establish a behavioural baseline with respect to behavioural parameters like activity, resting, scratching, grooming and shaking, all dogs were infested with 80 C. felis. Four days after infestation, fleas were removed and counted. A second infestation with 100 fleas was performed 2 weeks after the first infestation, followed by the removal and counting of fleas. Behavioral parameters during the days of infestation were compared to the established behavioural baseline. A third infestation with 120 fleas was planned in case an infestation with 100 fleas was not sufficient to lead to significant behavioral changes in comparison to the behavioural baseline.

Following the first infestation with 80 fleas, the number of fleas at assessment and changes in behavioral parameters were not as noticeable in all dogs. Following the second infestation with 100 C. felis, some behaviors differed clearly from the established baseline. Significant changes were obtained in paired t-tests for grooming and resting (day and night), barking and high activity (day), scratching, shaking and low activity (night). Therefore, a third infestation with 120 fleas was omitted.

All dogs remained in good clinical health throughout the study.

As more fully described herein, at least an n infestation with 100 fleas provoked significant changes in behavioral parameters (for example grooming and resting at day and night).

Test item (test article): Animo® is an activity and behavior monitor device which learns and accurately interprets the unique patterns of a dog Animo® delivers insights into a dog's activity and sleep patterns, as well as behaviors such as shaking, scratching and barking, which are indicative of underlying problems (wellbeing). From the moment Animo® was attached to a dog's collar, its suite of adaptive algorithms began to learn the animal's unique patterns of movement that are specific for each dog; accurately interpreting them and reporting their corresponding activity and behavior types, e.g. to the SURE Petcare—Animo® smartphone app (hereinafter: “app”), or via any other output device through which notifications can be provided to the animal caregiver/vetrinarian.

### Test System:

| | |
|---|---|
| Species/breed: | Dog, Beagle. |
| Justification: | Dogs are the intended species for the use of the Animo ® |
| | device. |
| Origin: | Permanent dog colony of the study site. |
| Number of | 8 dogs, plus 2 reserve. |
| animals: | |
| Sex: | Male and female, neutered. |
| Body weight: | 10.4-14.9 kg on the day of inclusion. |
| | Body weights were rounded half up to the nearest 0.1 kg. |
| Age: | Adult, 2 to 3.5 years. |
| Identification: | Dogs were uniquely identified by individual transponders |
| | and names. |
| Requirements: | Clinically healthy animals as determined by the clinical |
| | examination prior to inclusion into the study on study |
| | day 1. |

### Study Procedures

### Animal Management, Feed and Water:

The dogs were kept fed, with toys, water, appropriate temperature and lighting conditions, socially stable groups of dogs were maintained.

### Animal Health

### Clinical Examination:

On the day of inclusion (study day 1) and on the last day of the recovery period (study day 26), all dogs were clinically examined by a veterinarian. The clinical examination included the measurement of the rectal body temperature and the assessment of the cardiovascular system (auscultation, capillary refill), respiratory system (auscultation), superficial lymph nodes (e.g. Lnn mandibulares) and signs of lameness or discomfort. Special attention was laid on skin and fur (e.g. alopecia, hair loss).

### General Health Observations (GHO):

From the start of acclimatization until the end of the animal phase, general health observations (general condition and appetite) were performed twice daily, in the morning and in addition to the study plan a second time in the afternoon.

Any abnormal observation was documented. Animals suffering ill-health or discomfort were clinically examined and treated upon decision of the study supervisor.

### Observation of Abnormal Reactions Towards Test Item:

During general health observations, the animals were also inspected for appropriate placement of collars and devices and possible reactions towards the test item or its “attachment”.

### Observation of Abnormal Reactions After Flea Infestation:

Following each infestation with fleas, the animals were continuously monitored for the first hour. For the following 4 days dogs were inspected for signs associated with local irritation and/or reaction, including erythema, flaking/scaling, dry skin, cracked skin, edema, alopecia, blistering, oozing, hives, and wheals. For each time point, each assessment parameter was scored as not existent (A), slight change (B), moderate change (C), or severe change (D).

### Group Allocation

From 10 dogs starting into the acclimatization phase (see table 1), 8 dogs were selected on the basis of clinical health, data recordings during the acclimatization phase (baseline) and behavior. Two dogs (“Flash” and “Pablo”) were identified as reserve animals, as they were more nervous in character as the other dogs. All 8 dogs participating in the study formed the study population. There was no differentiation into study groups. Thus, a randomization was not applicable.

### Administration of Test Item

All Animo® devices were administered to the collar of the individual animal according to the requirements of the manufacturer. The devices were administered to all dogs selected for the acclimatization period, 17 days prior to the first infestation (study day 0).

TABLE 1
Assignment of test items to study animals (n = 10)
Animal Name

Anton
Falk
Flash¹
Josie
Lolly
Pablo¹
Paul
Mable
Maggie
Zuendie
¹Reserve animals

### Infestation with Fleas

The first flea infestation was conducted on study day 0 (n=80 fleas). Based on the results of this first infestation, a second infestation was conducted on study day 14 with 100 fleas. A third infestation with 120 fleas was omitted.

All dogs were infested with parasites of the following kind: C. felis: vital, unfed, male and female adult fleas, age ≤4 weeks. Fleas were directly applied to the fur of each dog along the backline, body side and/or head.

### Assessment of Parasite Burden

96 hours after each infestation, fleas were removed and the number of fleas per dog was counted and recorded (i.e. parasite count).

For the parasite count, the whole body of each dog was carefully examined, and fleas were collected by combing dogs with a flea comb. Removed fleas were counted. The dogs were assessed according to non-systematic order as they “came to hand”.

### Data Collection and Processing

The data were collected by using the Animo® device (for allocation of animals and devices see Table 1). It is to be noted that other behavior characterizing devices, other than Animo® could be used, mutatis mutandis.

From the moment the Animo® was attached to a dog's collar, the Animo® continuously recorded the acceleration data. Each Animo® included a 3 axis accelerometer sensor and an integral non-volatile memory. The memory of the Animo® was able to store data of a period up to 2 weeks. Furthermore, each Animo® consisted of an implemented suite of algorithms that began to learn the animal's unique patterns of movement; accurately interpreting them into dog states (e.g. shaking, grooming, scratching, resting, sleeping, high-activity, medium activity, low-activity, barking, calories burned, walking, running, sitting, lying, jumping, chewing, sniffing, licking, etc.) and reporting their corresponding activity and behavior types to the smartphone (Apple iPhone) application (App) Animo®. Animo® connected to the SURE Petcare Animo® app via Bluetooth Low Energy (BLE), however this is not limiting and the connection can be established in other manners. Via the SURE Petcare app individual activity and behavior profiles were generated.

A period of at least 7 days was used for Animo® to learn the dog's “normal” levels of activity (its behavioral baseline), but other amounts of time could be used for collecting behavioral baseline data.

The data collection was a continuous process once the Animo was attached to the dog. The data collected at each meaningful time period (e.g. 10 seconds, 15 seconds, 30 seconds, 1 minute, etc.) was analyzed and classified as behaviors.

During the acclimatization period in this example the data of the Animo® were collected for 18 days and transferred to a data cloud environment. On basis of these data the normal behavior pattern, also referred to herein as “behavioral baseline” of each dog was defined.

During the animal phase, the data of time intervals, of continuously collected data, following infestation were selected for data evaluation in segments of 24 hours (09:00 am-09:00 on the following day) up to 4 days (24 hours). On basis of these data the behavioral pattern following infestation was determined (Infestation 1, Infestation 2).

### Animal Welfare Parameter

Depending on the data packages following the infestations the most suitable parameter to monitor the animal wellbeing were identified.

Key features of Animo® and the Sure Petcare app included:
Activity: Set and monitor daily activity goals and view activity reports by day, week, month and year.
Calories: Tracking of calories burnt by the dog and comparison to a recommended daily target based on the breed, age and weight
Sleep quality: Hour-by-hour sleep quality report throughout the night; a poor night's sleep can be an indication of stress, discomfort or illness.
Behavior tracking: Displays incidents of increased barking, scratching or shaking

### Activity

Animo® tracked the total time in hours and minutes that the dog was active each day. Activity was categorized as walking, running or any other movement, such as shaking.

### Calories

Animo® calories calculation was based on an industry standard calculation that takes into account the dog's weight. The calories burnt were tracked against each movement type of the dog. Beside body weight the app also took into account a neutered status and the age.

### Sleep Quality

Poor-quality sleep may be a sign of stress, discomfort, need for intervention, or illness. The app recognized if the dog's sleep quality was much lower than normal last night, or previous nights, and compared sleep quality data with the dog's average. The dog's sleeping hours were individually defined within the app (e.g. 0 pm-5 am). An individual dog's sleeping hours can be adjusted based on that dog's typical sleeping patterns and timing. As an alternative, a dog's sleeping hours can be determined, at least in part, based on the data collected by the Animo

### Tracking

Animo® accurately detected when the dog barked, scratched or shook. The amount of these behaviors was tracked by the device and compared with the normal or typical behavior (e.g. the baseline). Resting times of the dog were continuously tracked by the App during the day.

The following parameters were selected for characterization of the wellbeing of the dogs (wellbeing): resting, barking, grooming, scratching, shaking, low activity, mid activity, and high activity.

### Statistical Analysis

### Assessment of Adequacy of Infestation

Each dog participating in the study was considered to be adequately infested when about 50% of the number of fleas used for infestation were retrieved 4 days after infestation.

### Analysis of Animo® Data

The evaluation of the data for each parameter was performed on the basis of events per day.

The interpreted data from each infestation (“infested”) was compared to the behavioral baseline (“not infested”). After the first infestation with 80 fleas, smaller differences were detected compared to the data based on the 100 flea infestation.

The implemented algorithm of each Animo® continuously tracks a variety of behavioral states (e.g. shaking, grooming, scratching, resting, sleeping, high-activity, medium activity, low-activity, barking, calories burned, walking, running, sitting, lying, jumping, chewing, sniffing, licking, etc.). The following parameters were used for the statistical analysis of event numbers:
Rest low: The absolute number (accuracy of 0.01) and continuous duration of sleeping periods per 24 hours.
Rest high: The absolute number of events where the dogs head is not touching the ground.
Resting: Sum of rest high and rest low.
Barking: The absolute number of Events per 24 hours (09:00 am to 09:00 pm).
Grooming: The absolute number of Events per 24 hours (09:00 am to 09:00 pm), The time of the longest grooming period (Tmax), and the number of continuous grooming phases per 24 hours.
Scratching: The absolute number of Events per 24 hours (09:00 am to 09:00 pm), The time of the longest scratching period (Tmax), and the number of continuous scratching phases per 24 hours.
Shaking: The absolute number (accuracy of 0.01) of shaking periods (Events) per 24 hours (e.g. 09:00 am to 09:00 am).
Low activity: The absolute number of Events per 24 hours (09:00 am to 09:00 pm), The time of the longest period with low activity (Tmax), and the number of continuous low activity phases per 24 hours.
High activity: The absolute number of Events per 24 hours (09:00 am to 09:00 pm), The time of the longest period with high activity (Tmax), and the number of continuous high activity phases per 24 hours.
Times of day were defined as follows:
Day is the time from 7 a.m. until before 7 p.m.
Night is the time from 7 p.m. until before 7 a.m.

The observation period started on study day −16 and was stopped on study day 18 (9 a.m.), infestations were conducted on study day 0 (between 9 a.m. and 10 a.m.) and on study day 14 (between 9 a.m. and 10 a.m.). Fleas were removed after the first infestation

The days on which infestations were conducted or fleas were removed were not considered for the statistical analysis.
Infestation status was therefore defined as follows:
Not infested: study day −15 until study day −1, study day 5 until study day 13
Infested: study day 1 until study day 3, study day 15 until study day 17

Mean numbers of events per hour were determined for each dog, parameter, day, time of day and infestation status.

A generalized linear model was applied to investigate the following effects (α=0.05) on the mean numbers of events per hour:
Status (Infested/Not infested)
Time of day (Day/Night)
Status and time of day interaction

Additionally, mean numbers of events per hour were determined for each dog, parameter, time of day and infestation status.

A separate analysis was conducted for each time of day to investigate a possible influence of infestation status. A two-sided t-test for paired samples per dog (α=0.05) was used to compare the mean number of events during infested periods with the mean number of events during not infested periods.

### Results

Animal health

### Clinical Examination

At clinical examination on study day 1 and at the end of the animal phase (study day 26) no abnormalities were detected in any of the dogs (8 plus 2 reserve animals at inclusion).

### General Health Observations

General behavior and appetite were normal in all animals throughout the animal phase of the study.

### Observation of Abnormal Reactions After Flea Infestation

Four days after the first flea infestation (n=80 fleas), slight erythema was observed in three dogs (Anton, Mable, Paul) which remained until the day before the second infestation (n=100 fleas). For 2 days, the third and fourth days after the second infestation, slight erythema was observed in three dogs (Lolly, Mable, Maggie). Pruritus was observed in Lolly and Mable. Mable also showed local loss of hair four days after the second infestation (study day 18).

### Description of Study Population

### Determination of Bodyweights

The body weights of the 8 dogs included in the study were between 10.4 kg and 14.9 kg at study day 1 and between 10.0 kg and 14.4 kg at the end of the animal phase, with a loss in body weight of up to 5% in all but one dog.

TABLE 2
Individual body weights and summary of body weights

| | Body weight (kg) | Body weight (kg) |
|---|---|---|
| Animal Name | study day −1 | study day 26 |
| Anton | 14.3 | 13.9 |
| Falk | 11.9 | 11.7 |
| Flash | 12.2 | n.a. (reserve animal) |
| Josie | 10.4 | 10.5 |
| Lolly | 10.5 | 10.0 |
| Pablo | 13.7 | n.a. (reserve animal) |
| Paul | 14.9 | 14.4 |
| Mable | 12.5 | 12.1 |
| Maggie | 12.5 | 12.1 |
| Zuendie | 11.8 | 11.7 |
| Mean | 12.47 | 12.05 |
| Standard deviation | 1.48 | 1.50 |
| Minimum | 10.4 | 10.0 |
| Maximum | 14.9 | 14.4 |

### Distribution of Age and Sex

For the distribution of age and sex refer to Table 3.

TABLE 3
Individual age and sex of study animals

| | Age [years, | | |
|---|---|---|---|
| | rounded to | | |
| Animal Name | 0.5] | Date of birth | Sex |
| Anton | 2 | 26 Jun. 2018 | mn |
| Falk | 2 | 4 Jun. 2018 | mn |
| Josie | 3.5 | 13 Sep. 2016 | fn |
| Lolly | 2 | 3 May 2018 | fn |
| Paul | 2 | 5 Mar. 2018 | mr |
| Mable | 3.5 | 12 Sep. 2016 | fn |
| Maggie | 3.5 | 7 Sep. 2016 | fn |
| Zuendie | 3.5 | 24 Aug. 2016 | fn |
| Mean | 2.75 | | No. of females: |
| Standard deviation | 0.80 | | N = 05 |
| Minimum | 2 | | Number of males: |
| Maximum | 3.5 | | N = 03 |

mn: male neutered,
fn: fenmale neutered,
N: number

### Adequacy of Infection

Infestation was considered adequate, if 50% of the fleas were retrieved 4 days after infestation. Due to pair housing during the first infestation, the distribution of fleas became uneven within the two dogs until assessment. Thus, infestation was not sufficient in several dogs. Furthermore, the data collected of the animals for the animal welfare parameter, indicated no behavioral changes, that could be evaluated statistically.

During the second infestation period, the dogs were housed individually and the data collected were considered appropriate for statistical evaluation.

The individual numbers of fleas retrieved 4 days after the respective infestation and the individual dogs are given in Table 4.

TABLE 04
Numbers of fleas counted at assessment

| | No. of fleas | No. of fleas |
|---|---|---|
| Animal Name | (1^{st} infestation, n = 80) | (2^{nd} infestation, n = 100) |
| Anton | 82 | 46 |
| Falk | 15 | 58^{a} |
| Josie | 112 | 76 |
| Lolly | 80 | 96 |
| Paul | 107 | 71 |
| Mable | 6 | 54 |
| Maggie | 42 | 59 |
| Zuendie | 28 | 57 |

The flea number recollected from one animal (Anton) was slightly below the threshold of 50%. As the actual number of 46 fleas was only slightly below (<10%) the anticipated minimum number of 50 fleas in one individual animal, infestation was considered to be adequate for the study objective. It was decided to not exclude the data of Anton from further statistical evaluation.

### Animal Welfare Parameters

The mean of events per hour are summarized for each parameter and each combination of time of day and infestation status (after infestation with 100 fleas) in Table 5, the results for effects investigated with the generalized linear model are summarized in Table 6. The results for the paired t-tests conducted separately for each time of day are summarized in Table 7.

TABLE 5
Mean and standard deviations of mean events per hour

| | Day | Day | Night | Night |
|---|---|---|---|---|
| Parameter | Infested | Not infested | Infested | Not infested |
| Grooming | 2.51 ± 1.25 | 1.49 ± 0.55 | 1.41 ± 0.72 | 0.56 ± 0.16 |
| Barking | 0.87 ± 0.63 | 1.14 ± 0.64 | 0.10 ± 0.09 | 0.09 ± 0.03 |
| Scratching | 1.49 ± 0.76 | 1.74 ± 0.86 | 0.53 ± 0.15 | 0.34 ± 0.15 |
| Shaking | 0.51 ± 0.12 | 0.53 ± 0.11 | 0.30 ± 0.08 | 0.22 ± 0.05 |
| Resting | 46.98 ± 1.50 | 45.12 ± 2.22 | 54.70 ± 1.09 | 56.49 ± 0.62 |
| High activity | 2.71 ± 1.34 | 4.86 ± 1.87 | 0.55 ± 0.30 | 0.56 ± 0.30 |
| Low activity | 3.80 ± 0.85 | 4.08 ± 1.07 | 1.64 ± 0.53 | 1.16 ± 0.18 |

TABLE 6
Generalized linear model effects

| | Status | | |
|---|---|---|---|
| | (Infested/Not | Time of Day | Interaction |
| Parameter | infested) | (Day/Night) | Status × Time of Day |
| Grooming | p < 0.0001 | p < 0.0001 | p = 0.4464 |
| Barking | p = 0.1662 | p < 0.0001 | p = 0.1182 |
| Scratching | p = 0.7687 | p < 0.0001 | p = 0.0194 |
| Shaking | p = 0.1605 | p < 0.0001 | p = 0.0102 |
| Resting | p = 0.1060 | p < 0.0001 | p < 0.0001 |
| High activity | p < 0.0001 | p < 0.0001 | p < 0.0001 |
| Low activity | p = 0.0130 | p < 0.0001 | p = 0.0042 |

TABLE 7
Results of paired t-tests for each day time

| | Day | Night |
|---|---|---|
| Parameter | Infested vs. not infested | Infested vs. not infested |
| Grooming | p = 0.0017 | p = 0.0069 |
| Barking | p = 0.0017 | p = 0.5912 |
| Scratching | p = 0.3185 | p = 0.0066 |
| Shaking | p = 0.3305 | p = 0.0098 |
| Resting | p = 0.0289 | p = 0.0017 |
| High activity | p = 0.0004 | p = 0.6968 |
| Low activity | p = 0.5202 | p = 0.0247 |

The generalized linear model revealed a highly significant difference between day and nighttime observations for all parameters (p<0.0001, written in bold).

For grooming, a significant difference between infested and not-infested study periods was observed (p<0.0001), this difference was similar at day and nighttime, hence no significant interaction was observed. The separate analysis for day and nighttime events confirms this observation (Day: p=0.0017, Night: p=0.0069). A graphical display of the observed mean number of events is given in FIG. 10a discussed above.

The difference between infested and not-infested study periods was not significant for barking and no significant interaction between status and time of day was observed. However, the separate analysis of day and nighttime events revealed a significant difference between infested and not infested study periods at daytime (p=0.0017). A graphical display of the observed mean number of events is given in FIG. 10b discussed above.

The difference between infested and not-infested study periods was not significant for scratching, but due to slightly more scratching in day time when not infested and slightly more scratching at night time when infested, the interaction between status and time of day was significant (p=0.0194). The separate analysis of day and nighttime events confirmed that the difference at nighttime was significant (p=0.0066). A graphical display of the observed mean number of events is given in FIG. 10c discussed above.

The difference between infested and not-infested study periods was not significant for shaking, but due to no difference at daytime, but slightly more shaking at nighttime when infested, the interaction between status and time of day was significant (p=0.0102). The separate analysis of day and nighttime events confirmed that the difference at nighttime was significant (p=0.0098). A graphical display of the observed mean number of events is given in FIG. 10d discussed above.

The difference between infested and not-infested study periods was not significant for resting, but due to slightly more resting in daytime when infested and slightly less resting at nighttime when infested, the interaction between status and time of day was significant (p<0.0001). When analyzing the day and nighttime events separately, a significant difference between infested and not-infested study periods is observed at day and night (Day: p=0.0298, Night: p=0.0017). A graphical display of the observed mean number of events is given in FIG. 10e discussed above.

For high activity, a significant difference between infested and not-infested study periods was observed (p<0.0001), this difference was very large at day time (more high activity when not infested), but not notable at night time, hence the interaction was also significant (p<0.0001). The separate analysis of day and nighttime events confirmed that the difference at day time was significant (p=0.0004). A graphical display of the observed mean number of events is given in FIG. 10f discussed above.

For low activity, a significant difference between infested and not-infested study periods was observed (p=0.0130). At daytime, more low activity was observed when not infested, at nighttime, more low activity was observed when infested, this interaction was also significant (p=0.0042). The separate analysis of day and nighttime events revealed no significant differences at day time, but a significant difference at night time (p=0.0247). A graphical display of the observed mean number of events is given in FIG. 10g discussed above.

### Discussion and Conclusions

As illustrated in this example study, recognizable changes in behavior of dogs, as monitored by the Animo device, can be provoked with the infestation of C. felis fleas. Changes become evident in grooming behavior, resting and activity patterns, especially at night. For example, an infestation dose of 100 fleas produced the trackable differences discussed above. Thus, the behaviors monitored by the Animo device may be used as surrogates for possible changes in the wellbeing of dogs.

### Example Study 2

Glossary of abbreviations and definition of terms

| | |
|---|---|
| am | At morning |
| Animo | Animo ® device (Animo ®) by Sure Petcare |
| BW | Body weight |
| CAS | Chemical Abstracts Service |
| C. felis | Ctenocephalides felis |
| D | Study day |
| Events | Distinguishable activities that are typically significantly shorter |
| | than a meaningful time period (e.g. 10 seconds, 15 seconds, 30 |
| | seconds, 1 minute, etc.) such as shaking, barking, scratching, |
| | jumping etc. |
| N | number |
| NA | Not applicable |
| pm | Past midday |
| Vs | Versus |

### Summary

This study evaluated a treatment effect of Bravecto® chewable tablets for dogs (Fluralaner) on the well-being of dogs, artificially infested with Ctenocephalides felis (C. felis) fleas (n=100).

Materials and Methods: A total of 12 healthy dogs carrying an Animo® device (Animo®) were included in the study. The dogs wore the Animo for 4 days— partly to establish baseline data. All dogs were infested with 100 C. felis.

Four days (96 hours) after infestation, all dogs were orally treated with the commercially available antiparasitic product Bravecto® chewable tablet for dogs (Fluralaner). The day of treatment was defined as study day 0. Seven days following treatment, the therapeutic treatment effect was assessed by removing all fleas from the animals and counting them.

For the assessment of the prophylactic treatment effect of Bravecto®, a second infestation with 100 C. felis was performed on study day 20. Before each infestation (study day −1 and study day 20) and for a period of four days following each infestation, skin and fur were examined for alterations/abnormal reactions. Behavioural parameters with respect to welfare like grooming, activity level, scratching, and shaking were assessed continuously by the Animo® from the beginning of acclimatisation until the end of the animal phase.

Statistical evaluations were performed on the following parameters: grooming, high activity, mid activity, low activity, resting, scratching, and shaking, comparing the periods: “untreated/baseline” (study day −8 to study day −4), “infested” (study day −4 to study day 0), “treated” (study day 0 to study day 4), and “protected” (study day 20 to study day 24).

### Results

All animals were healthy throughout the whole study. No adverse reactions were observed in all dogs after administration of the test item (Bravecto® chewable tablets for dogs (Fluralaner)) nor after administration of the Animo® Device (Animo)

Therapeutic efficacy against C. felis, assessed seven days after treatment, was 100%. Prophylactic efficacy against C. felis, assessed 24 days after treatment and 4 days after re-infestation with fleas was 100%.

The flea infestation had an impact on dog behaviour, including grooming at night time (p=0.0167) and resting at night time (p=0.0303), grooming at day time (p=0.1095), low activity at night time (p=0.0755) and scratching at night time (p=0.1242).

An immediate treatment effect on dog behaviour could not be observed, but a tendency was visible for low activity at night time (p=0.1343), resting at day time (p=0.1260) and resting at night time (p=0.0869).

A long-term treatment effect on dog behaviour was observed for grooming at night time (p=0.0161), low activity at night time (p=0.0412) and resting at night time (p=0.0001). These results illustrate that Bravecto® chewable tablets for dogs (Fluralaner) were well tolerated in all dogs participating in this study. When administered to dogs, artificially infested with Ctenocephalides felis (C. felis) fleas (n=100), (prophylactic) administration has a significant effect on grooming, resting, and scratching behavior as monitored by the Animo device. Differences in behavior as monitored by the Animo device are more distinct with prophylactic (as shown by “protected” period) than therapeutic (as shown by “treated” period) administration.

### Test Item (Test Article)

| | |
|---|---|
| Name: | Bravecto ® chewable tablet for dogs |
| Active | Fluralaner (AH252723). |
| ingredient: | |
| CAS no. | 864731-61-3. |
| (active | |
| ingredient): | |
| Formulation | Soft chew for medium size dogs (>10-20 kg BW). |
| details: | |
| Manufacturer: | Intervet GesmbH, Siemensstr. 107, 1210 Vienna, |
| | Austria. |
| Content of | 500 mg per soft chew. |
| active | |
| ingredient: | |
| Dose: | 25-50 mg Fluralaner/kg BW (500 mg per tablet). |
| Characterization | Soft chew for oral administration. The product |
| of test item: | information is included in the study file. |
| Storage | Keep medicines inaccessible for children. |
| conditions: | The test item will be stored at room temperature |
| | (i.e. 15-25° C.), dry and protected from light. |
| | The test item was a commercially available products |
| | that was tored according to legal and manufacturer |
| | requirements at the animal health service of MSD |
| | Animal Health Innovation GmbH until the day of |
| | treatment. |

### Test System

| | |
|---|---|
| Species/breed: | Dog/Beagle. |
| Justification: | Dogs are the intended species for treatment with |
| | Bravecto ® chewable tablet for dogs. |
| Origin: | Permanent dog colony of the study site. |
| Number of animals: | 12 dogs were included into the study. |
| Sex: | Male and female, neutered. |
| Body weight: | 10.0-16.5 kg on the day of inclusion (study day −6). |
| | Body weights were rounded to the nearest 0.1 kg. |
| Age: | Adult, 1.9 to 4.8 years. |
| Identification: | Dogs were uniquely identified by individual |
| | transponders and names. |
| Requirements: | Clinically healthy animals as determined by the |
| | clinical examination prior to inclusion into the |
| | study on study day −6. |
| Disposition/ | After the animal phase, dogs were returned to the |
| fate of animals: | resident dog colony |

### Study Animals

12 out of 15 animals wearing an Animo® were selected for the study on the day of the start of the acclimatization (study day −8).

TABLE 8
Animal details

| | | | Body weight on |
|---|---|---|---|
| | | | study day −6 |
| Animal Name | Sex | Date of birth | [kg] |
| Anton | Mn | 26 Jun. 2018 | 13.6 |
| Falk | Mn | 4 Jun. 2018 | 11.5 |
| Josie | Fn | 13 Sep. 2016 | 10.4 |
| Lolly | Fn | 3 May 2018 | 10.0 |
| Paul | Mn | 5 Mar. 2018 | 14.1 |
| Mable | Fn | 12 Sep. 2016 | 12.0 |
| Maggie | Fn | 7 Sep. 2016 | 12.3 |
| Sergeant Pepper | Mn | 28 Jul. 2015 | 15.3 |
| Rowdy | Mn | 28 Jul. 2015 | 14.6 |
| Buddha | Mn | 13 Sep. 2016 | 16.5 |
| Dumpty | Mn | 15 Mar. 2016 | 13.7 |
| Zuendie | Fn | 24 Aug. 2016 | 11.7 |

No.: Number,
ID: Identification,
D: Study day,
Mn: Male neutered,
Fn Female neutered

### Study Procedures

### Animal Management, Feed and Water

The dogs were kept fed, with toys, water, appropriate temperature and lighting conditions, socially stable groups of dogs were maintained.

### Animal Health

### Clinical Examination

On study day −6 and on the last day of the recovery period (study day 38), all dogs were clinically examined The clinical examination included the measurement of the rectal body temperature and the assessment of abnormalities of the cardiovascular system (auscultation, capillary refill), respiratory system (breathing quality). superficial lymph nodes (Lnn. mandibulares) and signs of lameness and discomfort. Special attention was laid on skin/fur (alopecia, hair loss). The examinations were performed by a veterinarian.

### General Health Observations (GHO)

General health observations (general condition and appetite) were performed twice daily from start of acclimatization until the end of the animal phase.

### Observation of Abnormal Reactions After Test Item Administration

Following administration of the test item and during general health observations, the animals were inspected for abnormal reactions or signs of illness (remaining of feed after feeding times).

### Observation of Abnormal Reactions After Flea Infestation

Following each infestation with fleas (study day 0 and study day 20), the animals were continuously monitored for the first hour and for the following 4 days, i.e. 24, 48, 72, and 96 hours after treatment. All dogs were inspected for signs associated with local irritation and/or reaction, including erythema, flaking/scaling, dry skin, cracked skin, edema, alopecia, blistering, oozing, hives, and wheals. For each time point, each assessment parameter was scored as not existent (A), slight change (B), moderate change (C), or severe change (D).

### Concomitant Medication

From the beginning of the study (study day −8) until the termination of the animal phase (study day 38), all dogs received no medications that might have interfered with the aim of the study. No animal had to be removed from the study, unexpectedly died or was euthanized during the study.

### Determination of Body Weights

Body weights were determined on the day of inclusion (study day −6), as well as on the day of the determination of the individual flea burden following the second infestation (study day 24). The weighing was performed after feeding and after the determination of the individual flea burden.

### Group Allocation

12 dogs, out of 15 dogs wearing an Animo®, were included into the study, selected on the basis of clinical health, previous study experiences and behavior within their social groups. All animals that participated in the study formed the study population. There was no differentiation into study groups. A randomization was not performed. All animals were uniquely identified by microchip number

### Administration of Test Item

| | |
|---|---|
| Administration | On the day of treatment (study day 0) all dogs received |
| (route of | one Bravecto ® chewable tablet for middle sized dogs |
| administration | (10.0-20.0 kg). |
| and procedure): | The soft chew was offered to the individual dog for |
| | voluntary uptake but the chewable tablet was not |
| | voluntarily taken up by the dogs. Therefore, it was |
| | directly placed into the mouth. |
| | The administration was performed prior to feeding. |
| Justification | The oral route is the intended route for the administration |
| for the route of | of the Bravecto ® chewable tablet. |
| administration: | |
| Dose: | 1 tablet (500 mg Fluralaner) per dog. |
| Administration | Once. |
| frequency: | |
| Administration | study day 0. |
| date: | |

TABLE 9
Individual doses

| | | | Dose | |
|---|---|---|---|---|
| | BW study | Dose | administered | Dose |
| Animal | day −6 | nominal | per dog | individual |
| Name | [kg] | [mg/kg BW] | [mg] | [mg/kg BW] |
| Anton | 13.6 | 25.0-50.0 | 500 | 36.8 |
| Falk | 11.6 | 25.0-50.0 | 500 | 43.1 |
| Josie | 10.4 | 25.0-50.0 | 500 | 48.1 |
| Lolly | 10.0 | 25.0-50.0 | 500 | 50.0 |
| Paul | 14.1 | 25.0-50.0 | 500 | 35.5 |
| Mable | 12.0 | 25.0-50.0 | 500 | 41.7 |
| Maggie | 12.3 | 25.0-50.0 | 500 | 40.7 |
| Sergeant | 15.3 | 25.0-50.0 | 500 | 32.7 |
| Pepper | | | | |
| Rowdy | 14.6 | 25.0-50.0 | 500 | 34.2 |
| Buddha | 16.5 | 25.0-50.0 | 500 | 30.3 |
| Dumpty | 13.7 | 25.0-50.0 | 500 | 36.5 |
| Zuendie | 11.7 | 25.0-50.0 | 500 | 42.7 |

### Infestation with Fleas

All dogs were infested twice with C. felis (n=100). The first infestation was performed on study day −4 and the second infestation on study day 20. All dogs were infested with parasites of the following kind: C. felis (isolate SHM 19): vital, unfed, male and female adult fleas, age ≤4 weeks. Fleas were directly applied to the fur of each unaffected, awake dog along the backline.

### Assessment of Flea Burden

Following each infestation, the individual flea burden of all dogs was determined.

The assessment following the first infestation was performed seven days after treatment (study day 7) to assess the therapeutic treatment effect of Bravecto®. The assessment following the second infestation was performed 96 h after the second infestation on study day 24, to assess the prophylactic treatment effect of Bravecto®. At these time points fleas were removed and the number of alive fleas per dog was counted and recorded (i.e. parasite count). For the parasite count, the whole body of each dog was carefully examined, and fleas were collected by combing dogs with a flea comb. Removed fleas were counted. The dogs were assessed according the non-systematic order as they came to hand. For the calculation of efficacy, the number of dead fleas was neglected.

### Data Collection and Processing

All data were collected by using the Animo® device. As discussed herein, Animo® is an activity and behavior monitor device, which learns and accurately interprets the unique patterns of a dog Animo® delivers insights into a dog's activity and sleep patterns, as well as behaviors such as shaking, scratching and barking, which may be indicative of underlying problems (wellbeing).

In this example, the data collection may be a continuous process once the device is attached to the collar. The data collection may also be near-continuous. Each interval of a meaningful time period (such as 10 seconds, 15 seconds, 30 seconds, 1 minute, etc.) is classified as a behavior.

Animo® can send alerts if the dog begins to show significant changes in behavior including barking, scratching, grooming, and/or shaking. Also, both long term and short term changes in the dog's sleep can be monitored. A decrease in quality may be a sign of a disease or other environmental factor which is disturbing the dog at night (wellbeing).

In this example, during the animal phase, the following time intervals, of continuously collected data, are predetermined for evaluation: Infestation 1, following the infestation up to treatment (study day −4 09:00 am — study day 0 09:00 am), Treatment 1 (therapeutic) (study day 0 09:00 am-study day 4 09:00 am), and Treatment 2 (prophylactic) (study day 20 09:00 am-study day 24 09:00 am).

### Animal Welfare Parameter

The following features of Animo® and the Sure Petcare app were included in those used to monitoring animal wellbeing following artificial flea infestation:

Activity: Set and monitor daily activity goals and view activity reports by day, week, month, and year. Behavior tracking: Displays incidents of increased grooming.

### Activity

Animo® tracked the total time in hours and minutes that the dogs were active each day, so it could be derived whether the animal got enough exercise to lead a healthy lifestyle. Activity was categorized as walking, running or any other movement, such as shaking.

### Behavior Tracking

Animo® accurately detected when the dog barked, scratched or shook. Significant increase in any of these behaviors was tracked by the device and compared with the normal behavior (also referred to herein as “behavioral baseline”). Resting times of the dog were continuously tracked by the App during the day.

In this example, the following parameters, alone or in combination, were selected for characterization of the wellbeing of the dogs: grooming, high activity, low activity, resting, scratching, shaking, and mid activity.

Additional parameters were not chosen in this example, but others could be used.

### Statistical Analysis

### Justification of Sample Size

The number of animals included into the study was 12 dogs. Ten dogs are sufficient to detect a difference in means of 0.5, assuming a standard deviation of 0.5, using a paired t-test with a power of 1−β=0.8 and a level of significance of α=0.025 (one-sided). This estimation corresponds to the results obtained for low activity at night in “Example Study 1”, detailed herinabove. This sample size is also large enough to detect larger differences as observed for grooming at day time (difference in means of 1.0, standard deviation of 0.9), grooming at night (difference in means of 0.85, standard deviation of 0.65) and high activity at day time (difference in means of 2.0, standard deviation of 1.0).

To make up for possible drop-outs due to inadequate infestation, 12 dogs were included into the study.

### Description of Study Group

Appropriate statistical parameters were used for a descriptive analysis of the study population with regards to initial age and body weight on study day −3 (clinical examination).

### Analysis of Animo® Data

The objective of this analysis was to investigate possible changes in individual behaviour with respect to flea infestation following the therapeutic treatment and concerning the prophylactic effect of Bravecto® chewable tablet.

The Animo® device recorded the following parameters on the basis of events (in meaningful time intervals such as 10 seconds, 15 seconds, 30 seconds, 1 minute, etc.) per day: Grooming, barking, scratching, shaking, resting, high, mid and low activity.

The following parameters were selected for evaluation:

### Primary Parameter:

Grooming: The absolute number of events.

### Secondary Parameters:

High activity: The absolute number of events.
Low activity: The absolute number of events.

Additional parameters were also monitored:
Resting: The absolute number of events
Scratching: The absolute number of events
Shaking: The absolute number of events
Mid activity: The absolute number of events

Events were summed up per hour.

Observations from 7:00 p.m. to 6:00 a.m. were categorized as “night time” observations, observations from 7:00 a.m. to 6:00 p.m. were categorized as “day time” observations. Day and night time observations will be analysed separately.

Observations were additionally categorized into study periods as follows:
“untreated period”: prior to the 1^{st} infestation on study day −4;
“infested period”: between the 1^{st} infestation on study day −4 and the 1^{st} treatment on study day 0;
“treated period”: between the 1^{st} treatment on study day 0 and study day 4;
“protected period”: between the 2^{nd} infestation on study day 20 and the determination of the individual flea burden on study day 24.

For each dog and parameter and time of day (night time/day time) and study period (untreated/infested/treated/protected period), the mean number of events per hour was determined.

For each parameter and time of day (night time/day time), study periods were compared pairwise using t-tests for paired observations (two-sided, α=0.05):
“infested period” vs. “untreated period” to confirm the effect of the flea infestation on dog behaviour
“infested period” vs. “treated period” to investigate a possible treatment effect on dog behaviour
“infested period” vs. “protected period” to investigate if the treatment effect on dog behaviour persists if the flea infestation is after treatment.

For grooming, high activity, low activity and resting, the mean number of events per hour was determined per study day (between study day −8 and study day 24) and time of day (night time/day time) and results were graphically displayed.

### Description of the Study Group

Twelve dogs were included into the study. There were no dropouts during the study period, so the data of all 12 dogs were used for the statistical analysis.

The mean age in the study group was 3.5±1.2 years, the age of the dogs ranged from 2 to 5 years. Mean body weight on study day −6 was 13.0±2.0 kg, body weight ranged from 10.0 to 16.5 kg.

Sexes were almost evenly distributed within the study group. Seven of the 12 dogs (58%) were male and neutered. Five of the 12 dogs (42%) were female and neutered.

### Animal Health

### Clinical Examination

At clinical examination on study day −6 no signs were observed in the animals that would have interfered with the study objective. Concerning skin and fur no alterations were observed and all animals were found to be healthy. Therefore, all animals were included into the study.

At the end of the animal phase (study day 38) all animals were examined again. All animals were healthy throughout the study.

### Observation of Abnormal Reactions After Test Item Administration

No abnormal reactions were observed in all animals after test item administration. No animal suffered ill-health or discomfort that needed veterinary assistance.

### Observation of Abnormal Reactions After Flea Infestation

In all animals, at all time points, before and after the infestations on study day 0 and study day 20, every parameter was examined and scored as A (no alterations).

### Concomitant Medication

From the beginning of the study until the termination of the animal phase, dogs received no medication which might have interfered with the aim of the study. In all dogs no live fleas were found following the second infestation with fleas. Therefore, no treatment with another licensed product against fleas was necessary.

### Determination of Body Weights

Body weights assessed on study day −6 and study day 24 are shown in table 10:

TABLE 10
Body weights

| | | Body weight on | Body weight on |
|---|---|---|---|
| | | study day −6 | study day −24 |
| Animal Name | Sex | [kg] | [kg] |
| Anton | Mn | 13.6 | 15.0 |
| Falk | Mn | 11.5 | 12.1 |
| Josie | Fn | 10.4 | 10.5 |
| Lolly | Fn | 10.0 | 10.8 |
| Paul | Mn | 14.1 | 14.9 |
| Mable | Fn | 12.0 | 12.6 |
| Maggie | Fn | 12.3 | 12.6 |
| Sergeant Pepper | Mn | 15.3 | 15.7 |
| Rowdy | Mn | 14.6 | 13.8 |
| Buddha | Mn | 16.5 | 16.5 |
| Dumpty | Mn | 13.7 | 13.8 |
| Zuendie | Fn | 11.7 | 11.7 |

No.: Number,
ID: Identification,
D: Study day,
Mn: Male neutered,
Fn: Female neutered

### Assessment of Parasite Burden

At assessments on study day 7 and study day 24 no live fleas were found in any of the dogs. On study day 24, in two dogs (Anton and Humpty) one and two (respectively) live fleas were found (see Table 11). Therapeutic efficacy against C. felis seven days after treatment was 100%. Prophylactic efficacy against C. felis 24 days after treatment and 4 days after reinfestation with fleas was 100%.

TABLE 11
Determination of flea numbers on study day 7 and study day 24

| | | Number of | | | Number of | | |
|---|---|---|---|---|---|---|---|
| Animal | Infestation | Fleas D 7 | | E_{(T)} | Fleas D 24 | | E_{(P)} |
| Name | dose | dead | live | [%] | dead | live | [%] |
| Anton | 100 | 0 | 0 | 100 | 0 | 1 | 100 |
| Falk | 100 | 0 | 0 | 100 | 0 | 0 | 100 |
| Josie | 100 | 0 | 0 | 100 | 0 | 0 | 100 |
| Lolly | 100 | 0 | 0 | 100 | 0 | 0 | 100 |
| Paul | 100 | 0 | 0 | 100 | 0 | 0 | 100 |
| Mable | 100 | 0 | 0 | 100 | 0 | 0 | 100 |
| Maggie | 100 | 0 | 0 | 100 | 0 | 0 | 100 |
| Sergeant | 100 | 0 | 0 | 100 | 0 | 0 | 100 |
| Pepper | | | | | | | |
| Rowdy | 100 | 0 | 0 | 100 | 0 | 0 | 100 |
| Buddha | 100 | 0 | 0 | 100 | 0 | 0 | 100 |
| Dumpty | 100 | 0 | 0 | 100 | 0 | 2 | 100 |
| Zuendie | 100 | 0 | 0 | 100 | 0 | 0 | 100 |

No.: Number,
D: Study day,
E_{(T)}: Therapeutic efficacy,
E_{(P)}: Prophylactic efficacy

### Animal Welfare Parameter

Detailed results obtained for each dog, time of day, study period and observed parameter are provided hereinbelow. Table 12 below summarizes the mean number of events per hour for each parameter, time of day and study period. After infestation, a clear increase in the primary parameter, grooming, was observed. During day time, the frequency of grooming further increased in the first days after treatment and the slowly declined. During night time, the frequency of grooming decreased after treatment and was comparable to pre-infestation during the protected period. No change was observed in high activity during night time. During day time, high activity decreased after infestation and further decreased after treatment, then increased again during the protected period.

A clear change was observed in low activity during night time. Low activity increased after infestation, then decreased after treatment and was comparable to pre-infestation during the protected period. No change was observed in low activity during day time. Of the additionally observed parameters, a clear change was seen in resting at night time. Time of resting was clearly reduced after infestation, was almost back to the pre-infestation duration after treatment and further increased in the protected period.

TABLE 12
Mean number of events per hour

| | | Untreated | Infested | Treated | Protected |
|---|---|---|---|---|---|
| | | period | period | period | period |
| | | (study day −8 | (study day −4 | (study day 0 | (study day 20 |
| | Time | to study | to study | to study | to study |
| Parameter | of day | day −4) | day 0) | day 4) | day 24) |
| Grooming | Day | 1.039 | 2.080 | 2.254 | 1.926 |
| | Night | 0.710 | 1.499 | 1.237 | 0.706 |
| High activity | Day | 5.046 | 3.852 | 3.016 | 3.949 |
| | Night | 0.515 | 0.551 | 0.526 | 0.540 |
| Low activity | Day | 2.973 | 2.896 | 2.818 | 2.709 |
| | Night | 1.090 | 1.641 | 1.179 | 1.004 |
| Resting | Day | 21.113 | 20.664 | 24.500 | 24.135 |
| | Night | 36.898 | 30.563 | 35.520 | 42.575 |
| Scratching | Day | 1.339 | 1.668 | 1.929 | 1.416 |
| | Night | 0.327 | 0.629 | 0.608 | 0.371 |
| Shaking | Day | 0.383 | 0.444 | 0.479 | 0.513 |
| | Night | 0.226 | 0.269 | 0.237 | 0.246 |
| Mid activity | Day | 0.124 | 0.150 | 0.084 | 0.095 |
| | Night | 0.025 | 0.068 | 0.042 | 0.040 |

These observations are confirmed by the results of the statistical analysis.

Table 13 summarizes the p-values that resulted from the comparison of the infested period to the untreated period, to the treated period, and to the protected period. Significant results on the α=0.05 level of significance (two-sided) are marked with an asterisk (*).

TABLE 13
Results of pairwise comparison of study periods

| | Time | Infested | Infested | Infested |
|---|---|---|---|---|
| Parameter | of day | vs. untreated | vs. treated | vs. protected |
| Grooming | Day | p = 0.1095 | p = 0.7870 | p = 0.8099 |
| | Night | p = 0.0167 | p = 0.4132 | p = 0.0161 * |
| High activity | Day | p = 0.1797 | p = 0.3442 | p = 0.9130 |
| | Night | p = 0.6891 | p = 0.7871 | p = 0.9078 |
| Low activity | Day | p = 0.9308 | p = 0.9292 | p = 0.8318 |
| | Night | p = 0.0755 | p = 0.1343 | p = 0.0412 * |
| Resting | Day | p = 0.8560 | p = 0.1260 | p = 0.1652 |
| | Night | p = 0.0303 * | p = 0.0869 | p = 0.0001 * |
| Scratching | Day | p = 0.5699 | p = 0.6527 | p = 0.6632 |
| | Night | p = 0.1242 | p = 0.9126 | p = 0.1871 |
| Shaking | Day | p = 0.1994 | p = 0.4565 | p = 0.1476 |
| | Night | p = 0.2097 | p = 0.3513 | p = 0.4954 |
| Mid activity | Day | p = 0.6771 | p = 0.2873 | p = 0.3749 |
| | Night | p = 0.3640 | p = 0.5772 | p = 0.5522 |

* Significant results on the α = 0.05 level of significance (two-sided)

The flea infestation impacted dog behaviour, including grooming at night time (p=0.0167), resting at night time (p=0.0303), grooming at day time (p=0.1095), low activity at night time (p=0.0755) and scratching at night time (p=0.1242).

After treatment, tendency was visible for low activity at night time (p=0.1343), resting at day time (p=0.1260) and resting lw at night time (p=0.0869).

A long-term treatment effect on dog behaviour was observed for grooming at night time (p=0.0161), low activity at night time (p=0.0412) and resting at night time (p=0.0001).

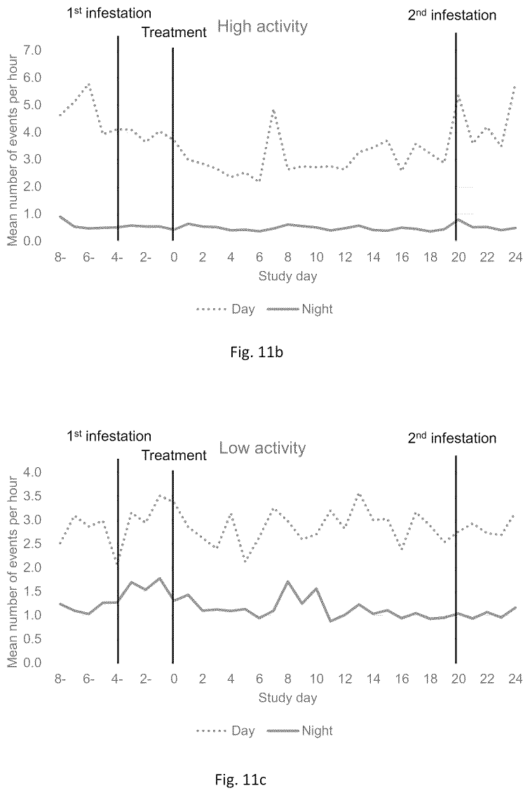

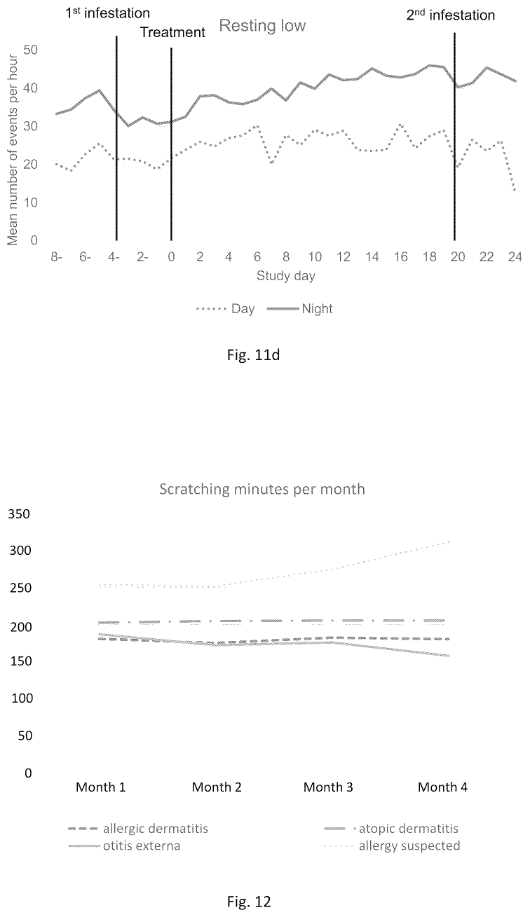

The course of the mean number of events per hour between study day −8 and study day 24 is displayed for both day time and night time results in FIG. 11a (Grooming), FIG. 11b (High activity), FIG. 11c (Low activity) and FIG. 11d (Resting lo).

### Discussion and Conclusions

Treatment with Bravecto® soft chew for middle sized dogs was well tolerated by all animals. No Adverse Events were observed throughout the animal phase.

As described above, statistically significant changes were observed for the animal welfare parameters grooming at night time and resting at night time. Grooming at nighttime: increase after flea infestation, decrease in protected period (study day 20 to study day 24) compared to infested period. Resting at nighttime: decrease after flea infestation, increase immediately after treatment, increase in protected period compared to infested period.

Changes were also observed for the animal welfare parameters grooming at day time and low activity at night time, and scratching at night time. Grooming at day time: increase after flea infestation. Low activity at nighttime: increase after flea infestation, decrease immediately after treatment, decrease in protected period compared to infested period. Scratching at nighttime: increase after flea infestation.

These results further illustrate that Animo® is suitable to detect changes in the animal welfare parameters of dogs when experimentally infested with Ctenocephalides felis, including grooming, resting, high activity, scratching, and low activity.

### Statistical Results:

Description of study group: sex, initial age, body weight:

| | | Body weight on | | Initial |
|---|---|---|---|---|
| Dog name | Sex | D −6 [kg] | Date of birth | age [years] |
| Anton | Male neutered | 13.6 | 26 Jun. 2018 | 2 |
| Falk | Male neutered | 11.5 | 4 Jun. 2018 | 2 |
| Josie | Female neutered | 10.4 | 13 Sep. 2016 | 4 |
| Lolly | Female neutered | 10 | 3 May 2018 | 2 |
| Mable | Female neutered | 12 | 12 Sep. 2016 | 4 |
| Maggie | Female neutered | 12.3 | 7 Sep. 2016 | 4 |
| Paul | Male neutered | 14.1 | 5 Mar. 2018 | 2 |
| Zuendie | Female neutered | 11.7 | 24 Aug. 2016 | 4 |
| Buddha | Male neutered | 16.5 | 13 Sep. 2016 | 4 |
| Dumpty | Male neutered | 13.7 | 15 Mar. 2016 | 4 |
| Rowdy | Male neutered | 14.6 | 28 Jul. 2015 | 5 |
| Sergeant Pepper | Male neutered | 15.3 | 28 Jul. 2015 | 5 |

| | Age [years] | Body weight [kg] |
|---|---|---|
| N | 12 | 12 |
| Mean | 3.5 | 13.0 |
| Std | 1.2 | 2.0 |
| Min | 2.0 | 10.0 |
| Median | 4.0 | 13.0 |
| Max | 5.0 | 16.5 |

| Sex | N | % |
|---|---|---|
| Female neutered | 5 | 41.7 |
| Male neutered | 7 | 58.3 |

### Mean Number of Events Per Hour for Each Dog, Time of Day, Study Period and Parameter

| | Grooming | | | | | | | |
|---|---|---|---|---|---|---|---|---|
| | Day | | | | Night | | | |
| Dog name | infested | protected | treated | untreated | infested | protected | treated | untreated |
| Anton | 0.28 | 0.30 | 0.42 | 0.12 | 0.95 | 0.40 | 0.51 | 0.37 |
| Buddha | 2.17 | 3.04 | 3.06 | 1.38 | 1.73 | 0.90 | 1.56 | 0.95 |
| Dumpty | 1.56 | 0.99 | 1.26 | 0.47 | 0.36 | 0.72 | 0.46 | 0.44 |
| Falk | 4.02 | 3.68 | 3.37 | 2.08 | 3.44 | 1.44 | 2.35 | 1.72 |
| Josie | 1.12 | 0.81 | 0.64 | 0.44 | 0.78 | 0.38 | 0.65 | 0.31 |
| Lolly | 2.87 | 3.23 | 2.56 | 1.24 | 1.67 | 0.55 | 1.13 | 0.53 |
| Mable | 3.30 | 1.38 | 2.69 | 2.05 | 2.44 | 0.76 | 1.77 | 1.27 |
| Maggie | 6.36 | 5.29 | 7.36 | 2.01 | 3.36 | 1.90 | 3.13 | 1.44 |
| Paul | 0.82 | 0.87 | 1.10 | 1.02 | 1.47 | 0.40 | 0.64 | 0.52 |
| Rowdy | 0.35 | 0.60 | 1.99 | 0.29 | 0.80 | 0.32 | 1.22 | 0.57 |
| Sergeant | 0.59 | 0.69 | 0.80 | 0.32 | 0.21 | 0.22 | 0.39 | 0.19 |
| Pepper | | | | | | | | |
| Zuendie | 1.53 | 2.22 | 1.79 | 1.05 | 0.76 | 0.48 | 1.06 | 0.21 |

| | High activity | | | | | | | |
|---|---|---|---|---|---|---|---|---|
| | Day | | | | Night | | | |
| Dog name | infested | protected | treated | untreated | infested | protected | treated | untreated |
| Anton | 6.16 | 5.66 | 3.92 | 7.12 | 0.63 | 0.43 | 0.49 | 0.46 |
| Buddha | 1.51 | 1.53 | 1.23 | 2.62 | 0.29 | 0.42 | 0.41 | 0.54 |
| Dumpty | 7.58 | 8.63 | 6.20 | 10.35 | 0.30 | 0.83 | 0.48 | 0.55 |
| Falk | 3.73 | 4.19 | 2.51 | 5.99 | 0.28 | 0.26 | 0.20 | 0.44 |
| Josie | 1.90 | 1.98 | 1.60 | 2.34 | 0.86 | 0.72 | 1.04 | 0.70 |
| Lolly | 5.01 | 5.53 | 4.02 | 8.05 | 0.81 | 0.80 | 0.55 | 0.61 |
| Mable | 2.41 | 2.22 | 2.13 | 3.75 | 0.28 | 0.37 | 0.19 | 0.24 |
| Maggie | 1.38 | 1.48 | 1.59 | 1.56 | 0.49 | 0.41 | 0.45 | 0.40 |
| Paul | 5.59 | 5.97 | 3.87 | 5.53 | 0.58 | 0.48 | 0.39 | 0.41 |
| Rowdy | 5.55 | 4.79 | 4.14 | 6.46 | 0.43 | 0.52 | 0.60 | 0.70 |
| Sergeant | 1.87 | 2.49 | 2.18 | 3.48 | 0.60 | 0.60 | 0.45 | 0.57 |
| Pepper | | | | | | | | |
| Zuendie | 3.53 | 2.90 | 2.81 | 3.30 | 1.06 | 0.63 | 1:07 | 0.55 |

| | Low activity | | | | | | | |
|---|---|---|---|---|---|---|---|---|
| | Day | | | | Night | | | |
| Dog name | infested | protected | treated | untreated | infested | protected | treated | untreated |
| Anton | 0.59 | 0.72 | 1.09 | 0.56 | 1.14 | 0.85 | 0.78 | 0.63 |
| Buddha | 2.63 | 1.68 | 3.31 | 2.07 | 1.46 | 0.65 | 0.96 | 1.47 |
| Dumpty | 3.20 | 1.59 | 1.92 | 2.17 | 2.16 | 0.92 | 1.23 | 2.03 |
| Falk | 3.74 | 3.43 | 3.64 | 3.57 | 1.29 | 0.80 | 0.76 | 1.31 |
| Josie | 7.73 | 7.39 | 6.40 | 9.34 | 4.45 | 2.15 | 3.36 | 2.96 |
| Lolly | 2.43 | 3.78 | 3.18 | 3.88 | 1.09 | 1.16 | 1.31 | 1.13 |
| Mable | 2.56 | 2.42 | 2.30 | 1.82 | 1.56 | 1.17 | 1.04 | 0.51 |
| Maggie | 5.78 | 5.26 | 4.41 | 7.85 | 1.27 | 1.06 | 0.86 | 0.62 |
| Paul | 1.94 | 2.52 | 2.72 | 1.71 | 1.86 | 0.80 | 1.15 | 0.86 |
| Rowdy | 0.95 | 1.22 | 1.41 | 1.23 | 1.10 | 1.04 | 0.90 | 0.74 |
| Sergeant | 1.28 | 1.26 | 1.33 | 0.85 | 0.81 | 0.59 | 0.46 | 0.35 |
| Pepper | | | | | | | | |
| Zuendie | 1.93 | 1.26 | 2.10 | 0.62 | 1.53 | 0.86 | 1.33 | 0.46 |

| | Resting low | | | | | | | |
|---|---|---|---|---|---|---|---|---|
| | Day | | | | Night | | | |
| Dog name | infested | protected | treated | untreated | infested | protected | treated | untreated |
| Anton | 19.23 | 17.96 | 23.61 | 16.74 | 19.56 | 39.36 | 29.78 | 29.98 |
| Buddha | 24.70 | 33.79 | 30.09 | 29.66 | 26.26 | 44.82 | 30.33 | 29.80 |
| Dumpty | 15.51 | 18.55 | 18.62 | 13.68 | 41.54 | 33.34 | 31.04 | 29.71 |
| Falk | 17.66 | 17.88 | 20.15 | 15.29 | 27.48 | 43.45 | 29.59 | 19.25 |
| Josie | 24.00 | 33.78 | 30.73 | 28.26 | 29.51 | 46.85 | 37.72 | 38.46 |
| Lolly | 23.14 | 24.58 | 27.24 | 20.56 | 30.46 | 46.22 | 40.92 | 46:01 |
| Mable | 17.79 | 22.21 | 20.39 | 19.15 | 29.57 | 43.10 | 40.45 | 44.21 |
| Maggie | 27.64 | 32.06 | 31.56 | 32.73 | 33.69 | 47.41 | 45.99 | 45.42 |
| Paul | 8.47 | 15.45 | 12.09 | 15.09 | 22.39 | 34.69 | 24.85 | 40.48 |
| Rowdy | 24.90 | 23.05 | 27.23 | 20.05 | 40.18 | 46.18 | 43.24 | 41.82 |
| Sergeant | 19.86 | 22.68 | 22.52 | 17.22 | 26.64 | 42.18 | 29.77 | 32.09 |
| Pepper | | | | | | | | |
| Zuendie | 25.05 | 27.62 | 29.77 | 24.92 | 39.50 | 43.30 | 42.57 | 45.54 |

| | Mid activity | | | | | | | |
|---|---|---|---|---|---|---|---|---|
| | Day | | | | Night | | | |
| Dog name | infested | protected | treated | untreated | infested | protected | treated | untreated |
| Anton | 0.00 | 0.01 | 0.00 | 0.01 | 0.00 | 0.00 | 0.00 | 0.01 |
| Buddha | 0.90 | 0.44 | 0.26 | 0.29 | 0.01 | 0.23 | 0.00 | 0.01 |
| Dumpty | 0.07 | 0.10 | 0.06 | 0.27 | 0.01 | 0.01 | 0.00 | 0.01 |
| Falk | 0.16 | 0.04 | 0.15 | 0.28 | 0.03 | 0.01 | 0.02 | 0.01 |
| Josie | 0.14 | 0.14 | 0.13 | 0.15 | 0.65 | 0.15 | 0.32 | 0.21 |
| Lolly | 0.12 | 0.06 | 0.05 | 0.14 | 0.01 | 0.01 | 0.00 | 0.01 |
| Mable | 0.02 | 0.02 | 0.03 | 0.08 | 0.02 | 0.01 | 0.01 | 0.00 |
| Maggie | 0.10 | 0.05 | 0.15 | 0.10 | 0.05 | 0.05 | 0.09 | 0.02 |
| Paul | 0.01 | 0.03 | 0.01 | 0.05 | 0.01 | 0.00 | 0.00 | 0.01 |
| Rowdy | 0.12 | 0.06 | 0.03 | 0.05 | 0.00 | 0.00 | 0.00 | 0.01 |
| Sergeant | 0.01 | 0.06 | 0.00 | 0.02 | 0.00 | 0.02 | 0.01 | 0.00 |
| Pepper | | | | | | | | |
| Zuendie | 0.14 | 0.13 | 0.13 | 0.06 | 0.04 | 0.01 | 0.06 | 0.02 |

### Comparison of Study Periods, Time: Day, Parameter: Grooming

| R-Square | Coeff Var | Root MSE | Grooming Mean |
|---|---|---|---|
| 0.089306 | 85.57875 | 1.561547 | 1.824690 |

| Source | DF | Type III SS | Mean Square | F Value | Pr > F |
|---|---|---|---|---|---|
| Status | 3 | 10.52129782 | 3.50709927 | 1.44 | 0.2445 |

| | |
|---|---|
| Level of | Grooming |

| Status | N | Mean | Std Dev |
|---|---|---|---|
| infested | 12 | 2.08023050 | 1.80158313 |
| protected | 12 | 1.92597518 | 1.56333868 |
| treated | 12 | 2.25354610 | 1.88021102 |
| untreated | 12 | 1.03900709 | 0.72717873 |

### Dependent Variable: Grooming Grooming

| | | | | F | |
|---|---|---|---|---|---|
| Contrast | DF | Contrast SS | Mean Square | Value | Pr > F |
| Infested vs. Untreated | 1 | 6.50487707 | 6.50487707 | 2.67 | 0.1095 |
| Infested vs. Treated | 1 | 0.18022979 | 0.18022979 | 0.07 | 0.7870 |
| Infested vs. Protected | 1 | 0.14276822 | 0.14276822 | 0.06 | 0.8099 |

### Comparison of Study Periods, Time: Night, Parameter: Grooming

| R-Square | Coeff Var | Root MSE | Grooming Mean |
|---|---|---|---|
| 0.175436 | 74.76874 | 0.776002 | 1.037869 |

| Source | DF | Type III SS | Mean Square | F Value | Pr > F |
|---|---|---|---|---|---|
| Status | 3 | 5.63733080 | 1.87911027 | 3.12 | 0.0354 |

| | |
|---|---|
| Level of | Grooming |

| Status | N | Mean | Std Dev |
|---|---|---|---|
| infested | 12 | 1.49869792 | 1.08867221 |
| protected | 12 | 0.70572917 | 0.50173556 |
| [symbol] | [symbol] | [symbol] | [symbol] 520 |
| [symbol] | [symbol] | [symbol] | [symbol] 829 |

[symbol]

indicates data missing or illegible when filed

### Dependent Variable: Grooming Grooming

| | | | | F | |
|---|---|---|---|---|---|
| Contrast | DF | Contrast SS | Mean Square | Value | Pr > F |
| Infested vs. Untreated | 1 | 3.73160920 | 3.73160920 | 6.20 | 0.0167 |
| Infested vs. Treated | 1 | 0.41098022 | 0.41098022 | 0.68 | 0.4132 |
| Infested vs. Protected | 1 | 3.77279663 | 3.77279663 | 6.27 | 0.0161 |

### Comparison of Study Periods, Time: Day, Parameter: High Activity

| R-Square | Coeff Var | Root MSE | High Mean |
|---|---|---|---|
| 0.109964 | 54.06451 | 2.143948 | 3.965536 |

| Source | DF | Type III SS | Mean Square | F Value | Pr > F |
|---|---|---|---|---|---|
| Status | 3 | 24.98747253 | 8.32915751 | 1.81 | 0.1589 |

| | |
|---|---|
| Level of | High |

| Status | N | Mean | Std Dev |
|---|---|---|---|
| infested | 12 | 3.85239362 | 2.08884829 |
| protected | 12 | 3.94858156 | 2.22068788 |
| [symbol] | [symbol] | [symbol] | [symbol] |
| [symbol] | [symbol] | [symbol] | [symbol] |

[symbol]

indicates data missing or illegible when filed

### Dependent Variable: High High Activity

| | | | | F | |
|---|---|---|---|---|---|
| Contrast | DF | Contrast SS | Mean Square | Value | Pr > F |
| Infested vs. Untreated | 1 | 8.54325109 | 8.54325109 | 1.86 | 0.1797 |
| Infested vs. Treated | 1 | 4.20220311 | 4.20220311 | 0.91 | 0.3442 |
| Infested vs. Protected | 1 | 0.05551272 | 0.05551272 | 0.01 | 0.9130 |

### Comparison of Study Periods, Time: Night, Parameter: High Activity

| R-Square | Coeff Var | Root MSE | High Mean |
|---|---|---|---|
| 0.004219 | 41.09820 | 0.219092 | 0.533095 |

| Source | DF | Type III SS | Mean Square | F Value | Pr > F |
|---|---|---|---|---|---|
| Status | 3 | 0.00894900 | 0.00298300 | 0.06 | 0.9795 |

| | |
|---|---|
| Level of | High |

| Status | N | Mean | Std Dev |
|---|---|---|---|
| infested | 12 | 0.55078125 | 0.25798223 |
| protected | 12 | 0.54036458 | 0.17894141 |
| treated | 12 | 0.52647569 | 0.27554055 |
| untreated | 12 | 0.51475694 | 0.13231876 |

### Dependent Variable: High High Activity

| | | | | F | |
|---|---|---|---|---|---|
| Contrast | DF | Contrast SS | Mean Square | Value | Pr > F |
| Infested vs. Untreated | 1 | 0.00778650 | 0.00778650 | 0.16 | 0.6891 |
| Infested vs. Treated | 1 | 0.00354456 | 0.00354456 | 0.07 | 0.7871 |
| Infested vs. Protected | 1 | 0.00065104 | 0.00065104 | 0.01 | 0.9078 |

### Comparison of Study Periods, Time: Day, Parameter: Low Activity

| R-Square | Coeff Var | Root MSE | Low Mean |
|---|---|---|---|
| 0.002251 | 75.46728 | 2.150031 | 2.848958 |

| Source | DF | Type III SS | Mean Square | F Value | Pr > F |
|---|---|---|---|---|---|
| Status | 3 | 0.45883495 | 0.15294498 | 0.03 | 0.9918 |

| | |
|---|---|
| Level of | Low |

| Status | N | Mean | Std Dev |
|---|---|---|---|
| infested | 12 | 2.89627660 | 2.05713322 |
| protected | 12 | 2.70877660 | 1.97410878 |
| treated | 12 | 2.81781915 | 1.51515576 |
| untreated | 12 | 2.97296099 | 2.84006008 |

### Dependent Variable: Low Low Activity

| | | | | F | |
|---|---|---|---|---|---|
| Contrast | DF | Contrast SS | Mean Square | Value | Pr > F |
| Infested vs. Untreated | 1 | 0.03528298 | 0.03528298 | 0.01 | 0.9308 |
| Infested vs. Treated | 1 | 0.03693343 | 0.03693343 | 0.01 | 0.9292 |
| Infested vs. Protected | 1 | 0.21093750 | 0.21093750 | 0.05 | 0.8318 |

### Comparison of Study Periods, Time: Night, Parameter: Low Activity

| R-Square | Coeff Var | Root MSE | Low Mean |
|---|---|---|---|
| 0.107187 | 60.40003 | 0.742155 | 1.228733 |

| Source | DF | Type III SS | Mean Square | F Value | Pr > F |
|---|---|---|---|---|---|
| Status | 3 | 2.90953686 | 0.96984562 | 1.76 | 0.1686 |

| | |
|---|---|
| Level of | Low |

| Status | N | Mean | Std Dev |
|---|---|---|---|
| infested | 12 | 1.64149306 | 0.95616632 |
| protected | 12 | 1.00434028 | 0.40293932 |
| [symbol] | [symbol] | [symbol] | [symbol] |
| [symbol] | [symbol] | [symbol] | [symbol] |

[symbol]

indicates data missing or illegible when filed

### Dependent Variable: Low Low Activity

| | | | | F | |
|---|---|---|---|---|---|
| Contrast | DF | Contrast SS | Mean Square | Value | Pr > F |
| Infested vs. Untreated | 1 | 1.82590174 | 1.82590174 | 3.32 | 0.0755 |
| Infested vs. Treated | 1 | 1.28199259 | 1.28199259 | 2.33 | 0.1343 |
| Infested vs. Protected | 1 | 2.43578197 | 2.43578197 | 4.42 | 0.0412 |

### Comparison of Study Periods, Time: Day, Parameter: Resting

| R-Square | Coeff Var | Root MSE | RestLow Mean |
|---|---|---|---|
| 0.082235 | 26.65740 | 6.025300 | 22.60273 |

| Source | DF | Type III SS | Mean Square | F Value | Pr > F |
|---|---|---|---|---|---|
| Status | 3 | 143.1313300 | 47.7104433 | 1.31 | 0.2818 |

| | |
|---|---|
| Level of | RestLow |

| Status | N | Mean | Std Dev |
|---|---|---|---|
| infested | 12 | 20.6635638 | 5.34870710 |
| protected | 12 | 24.1347518 | 6.41253366 |
| treated | 12 | 24.5000000 | 5.96686833 |
| untreated | 12 | 21.1125887 | 6.31539366 |

### Dependent Varialphe: RestLow Resting Low

| | | | | F | |
|---|---|---|---|---|---|
| Contrast | DF | Contrast SS | Mean Square | Value | Pr > F |
| Infested vs. Untreated | 1 | 1.20973975 | 1.20973975 | 0.03 | 0.8560 |
| Infested vs. Treated | 1 | 88.30945493 | 88.30945493 | 2.43 | 0.1260 |
| Infested vs. Protected | 1 | 72.29487442 | 72.29487442 | 1.99 | 0.1652 |

### Comparison of Study Periods, Time: Night, Parameter: Resting

| R-Square | Coeff Var | Root MSE | RestLow Mean |
|---|---|---|---|
| 0.293439 | 19.05487 | 6.933895 | 36.38911 |

| Source | DF | Type III SS | Mean Square | F Value | Pr > F |
|---|---|---|---|---|---|
| Status | 3 | 878.5660739 | 292.8553580 | 6.09 | 0.0015 |

| | |
|---|---|
| Level of | RestLow |

| Status | N | Mean | Std Dev |
|---|---|---|---|
| infested | 12 | 30.5633681 | 6.98079641 |
| protected | 12 | 42.5746528 | 4.59602792 |
| treated | 12 | 35.5203993 | 7.00191181 |
| untreated | 12 | 36.8980035 | 8.56935601 |

### Dependent Variable: RestLow Resting Low

| | | | | F | |
|---|---|---|---|---|---|
| Contrast | DF | Contrast SS | Mean Square | Value | Pr > F |
| Infested vs. Untreated | 1 | 240.7656352 | 240.7656352 | 5.01 | 0.0303 |
| Infested vs. Treated | 1 | 147.4329529 | 147.4329529 | 3.07 | 0.0869 |
| Infested vs. Protected | 1 | 865.6257641 | 865.6257641 | 18.00 | 0.0001 |

### Comparison of Study Periods, Time: Day, Parameter: Scratching

| R-Square | Coeff Var | Root MSE | Scratch Mean |
|---|---|---|---|
| 0.028586 | 88.72684 | 1.409168 | 1.588209 |

| Source | DF | Type III SS | Mean Square | F Value | Pr > F |
|---|---|---|---|---|---|
| Status | 3 | 2.57118134 | 0.85706045 | 0.43 | 0.7314 |

| | |
|---|---|
| Level of | Scratch |

| Status | N | Mean | Std Dev |
|---|---|---|---|
| infested | 12 | 1.66843972 | 1.44080193 |
| protected | 12 | 1.41622340 | 1.10960077 |
| [symbol] | [symbol] | [symbol] | [symbol] 133 |
| [symbol] | [symbol] | [symbol] | [symbol] 151 |

[symbol]

indicates data missing or illegible when filed

### Dependent Variable: Scratch Scratching

| | | | | F | |
|---|---|---|---|---|---|
| Contrast | DF | Contrast SS | Mean Square | Value | Pr > F |
| Infested vs. Untreated | 1 | 0.65080471 | 0.65080471 | 0.33 | 0.5699 |
| Infested vs. Treated | 1 | 0.40759393 | 0.40759393 | 0.21 | 0.6527 |
| Infested vs. Protected | 11 | 0.38167841 | 0.38167841 | 0.19 | 0.6632 |

### Comparison of Study Periods, Time: Night, Parameter: Scratching

| R-Square | Coeff Var | Root MSE | Scratch Mean |
|---|---|---|---|
| 0.082872 | 97.63410 | 0.472068 | 0.483507 |

| Source | DF | Type III SS | Mean Square | F Value | Pr > F |
|---|---|---|---|---|---|
| Status | 3 | 0.88600893 | 0.29533631 | 1.33 | 0.2783 |

| | |
|---|---|
| Level of | Scratch |

| Status | N | Mean | Std Dev |
|---|---|---|---|
| infested | 12 | 0.62890625 | 0.57148204 |
| protected | 12 | 0.37065972 | 0.24808723 |
| treated | 12 | 0.60763889 | 0.64963801 |
| untreated | 12 | 0.32682292 | 0.28499649 |

### Dependent Variable: Scratch Scratching

| | | | | F | |
|---|---|---|---|---|---|
| Contrast | DF | Contrast SS | Mean Square | Value | Pr > F |
| Infested vs. Untreated | 1 | 0.54752604 | 0.54752604 | 2.46 | 0.1242 |
| Infested vs. Treated | 1 | 0.00271380 | 0.00271380 | 0.01 | 0.9126 |
| Infested vs. Protected | 1 | 0.40014761 | 0.40014761 | 1.80 | 0.1871 |

### Comparison of Study Periods, Time: Night, Parameter: Shaking

| R-Square | Coeff Var | Root MSE | Shaking Mean |
|---|---|---|---|
| 0.038225 | 34.17863 | 0.083518 | 0.244358 |

| Source | DF | Type III SS | Mean Square | F Value | Pr > F |
|---|---|---|---|---|---|
| Status | 3 | 0.01219799 | 0.00406600 | 0.58 | 0.6294 |

| | |
|---|---|
| Level of | Shaking |

| Status | N | Mean | Std Dev |
|---|---|---|---|
| infested | 12 | 10.26909722 | 0.10331884 |
| protected | 12 | 0.24565972 | 0.07954311 |
| treated | 12 | 10.23697917 | 0.08420180 |
| untreated | 12 | 0.22569444 | 0.06171913 |

### Dependent Variable: Shaking Shaking

| Contrast | DF | Contrast SS | Mean Square | F Value | Pr > F |
|---|---|---|---|---|---|
| Infested vs. | 1 | 0.01130281 | 0.01130281 | 1.62 | 0.2097 |
| Untreated | | | | | |
| Infested vs. | 1 | 0.00618942 | 0.00618942 | 0.89 | 0.3513 |
| Treated | | | | | |
| Infested vs. | 1 | 0.00329590 | 0.00329590 | 0.47 | 0.4954 |
| Protected | | | | | |

### Comparison of Study Periods, Time: Day, Parameter: Mid Activity

| R-Square | Coeff Var | Root MSE | Mid Mean |
|---|---|---|---|
| 0.031181 | 132.7571 | 0.150205 | 0.113143 |

| Source | DF | Type III SS | Mean Square | F Value | Pr > F |
|---|---|---|---|---|---|
| Status | 3 | 0.03194967 | 0.01064989 | 0.47 | 0.7033 |

| | |
|---|---|
| Level of | Mid |

| Status | N | Mean | Std Dev |
|---|---|---|---|
| infested | 12 | 0.14982270 | 0.24455292 |
| protected | 12 | 0.09485816 | 0.11664334 |
| treated | 12 | 0.08377660 | 0.08083213 |
| untreated | 12 | 0.12411348 | 0.10149134 |

### Dependent Variable: Mid Mid Activity

| Contrast | DF | Contrast SS | Mean Square | F Value | Pr > F |
|---|---|---|---|---|---|
| Infested vs. | 1 | 0.00396578 | 0.00396578 | 0.18 | 0.6771 |
| Untreated | | | | | |
| Infested vs. | 1 | 0.02617252 | 0.02617252 | 1.16 | 0.2873 |
| Treated | | | | | |
| Infested vs. | 1 | 0.01812660 | 0.01812660 | 0.80 | 0.3749 |
| Protected | | | | | |

### Comparison of Study Periods, Time: Night, Parameter: Mid Activity

| R-Square | Coeff Var | Root MSE | Mid Mean |
|---|---|---|---|
| 0.019444 | 260.3842 | 0.113579 | 0.043620 |

| Source | DF | Type III SS | Mean Square | F Value | Pr > F |
|---|---|---|---|---|---|
| Status | 3 | 0.01125533 | 0.00375178 | 0.29 | 0.8318 |

| | |
|---|---|
| Level of | Mid |

| Status | N | Mean | Std Dev |
|---|---|---|---|
| infested | 12 | 0.06770833 | 0.18435654 |
| protected | 12 | 0.03993056 | 0.07319796 |
| treated | 12 | 0.04166667 | 0.09332818 |
| untreated | 12 | 0.02517361 | 0.05954311 |

### Dependent Variable: Mid Mid Activity

| Contrast | DF | Contrast SS | Mean Square | F Value | Pr > F |
|---|---|---|---|---|---|
| Infested vs. | 1 | 0.01085522 | 0.01085522 | 0.84 | 0.3640 |
| Untreated | | | | | |
| Infested vs. | 1 | 0.00406901 | 0.00406901 | 0.32 | 0.5772 |
| Treated | | | | | |
| Infested vs. | 1 | 0.00462963 | 0.00462963 | 0.36 | 0.5522 |
| Protected | | | | | |

### Daily Means Per Study Day and Time of Day

| Study | | Grooming | | High activity | | Low activity | | Resting low | |
|---|---|---|---|---|---|---|---|---|---|
| day | Date | Day | Night | Day | Night | Day | Night | Day | Night |
| −8 | 18 May 2020 | 1.123 | 0.921 | 4.630 | 0.892 | 2.512 | 1.233 | 20.046 | 33.292 |
| −7 | 19 May 2020 | 1.342 | 0.917 | 5.118 | 0.533 | 3.089 | 1.095 | 18.314 | 34.401 |
| −6 | 20 May 2020 | 0.708 | 0.625 | 5.783 | 0.467 | 2.861 | 1.024 | 22.670 | 37.392 |
| −5 | 21 May 2020 | 1.054 | 0.602 | 3.920 | 0.488 | 2.977 | 1.262 | 25.552 | 39.389 |
| −4 | 22 May 2020 | 0.797 | 0.859 | 4.106 | 0.505 | 2.063 | 1.264 | 21.396 | 34.313 |
| −3 | 23 May 2020 | 2.622 | 1.604 | 4.094 | 0.573 | 3.153 | 1.691 | 21.486 | 30.109 |
| −2 | 24 May 2020 | 2.090 | 1.283 | 3.642 | 0.538 | 2.939 | 1.533 | 20.865 | 32.323 |
| −1 | 25 May 2020 | 2.564 | 1.422 | 4.036 | 0.536 | 3.505 | 1.774 | 18.734 | 30.696 |
| 0 | 26 May 2020 | 2.492 | 1.620 | 3.703 | 0.422 | 3.369 | 1.302 | 21.595 | 31.179 |
| 1 | 27 May 2020 | 1.918 | 1.580 | 2.986 | 0.637 | 2.851 | 1.424 | 23.844 | 32.524 |
| 2 | 28 May 2020 | 2.743 | 1.293 | 2.844 | 0.538 | 2.622 | 1.095 | 25.974 | 37.887 |
| 3 | 29 May 2020 | 1.792 | 1.033 | 2.658 | 0.517 | 2.391 | 1.118 | 24.719 | 38.182 |
| 4 | 30 May 2020 | 2.793 | 0.823 | 2.352 | 0.398 | 3.142 | 1.087 | 26.944 | 36.316 |
| 5 | 31 May 2020 | 1.585 | 1.005 | 2.523 | 0.420 | 2.120 | 1.128 | 27.658 | 35.814 |
| 6 | 01 Jun. 2020 | 2.411 | 0.969 | 2.163 | 0.359 | 2.642 | 0.936 | 30.283 | 37.009 |
| 7 | 02 Jun. 2020 | 1.659 | 0.692 | 4.858 | 0.460 | 3.252 | 1.093 | 19.996 | 39.881 |
| 8 | 03 Jun. 2020 | 2.581 | 1.169 | 2.630 | 0.612 | 2.974 | 1.706 | 27.734 | 36.802 |
| 9 | 04 Jun. 2020 | 1.703 | 0.951 | 2.745 | 0.555 | 2.591 | 1.242 | 25.102 | 41.497 |
| 10 | 05 Jun. 2020 | 1.917 | 0.979 | 2.719 | 0.505 | 2.695 | 1.557 | 29.063 | 39.880 |
| 11 | 06 Jun. 2020 | 2.393 | 0.615 | 2.747 | 0.393 | 3.195 | 0.870 | 27.583 | 43.591 |
| 12 | 07 Jun. 2020 | 2.823 | 0.893 | 2.633 | 0.474 | 2.818 | 1.003 | 28.857 | 42.138 |
| 13 | 08 Jun. 2020 | 2.924 | 1.081 | 3.268 | 0.568 | 3.573 | 1.224 | 23.799 | 42.438 |
| 14 | 09 Jun. 2020 | 1.768 | 1.029 | 3.438 | 0.411 | 2.997 | 1.026 | 23.542 | 45.208 |
| 15 | 10 Jun. 2020 | 2.594 | 0.893 | 3.698 | 0.380 | 3.021 | 1.104 | 23.831 | 43.305 |
| 16 | 11 Jun. 2020 | 1.359 | 0.833 | 2.568 | 0.495 | 2.378 | 0.934 | 30.759 | 42.858 |
| 17 | 12 Jun. 2020 | 2.233 | 0.792 | 3.576 | 0.450 | 3.161 | 1.042 | 24.252 | 43.705 |
| 18 | 13 Jun. 2020 | 1.630 | 0.576 | 3.219 | 0.356 | 2.877 | 0.922 | 27.372 | 45.998 |
| 19 | 14 Jun. 2020 | 1.507 | 0.665 | 2.872 | 0.434 | 2.530 | 0.946 | 29.023 | 45.580 |
| 20 | 15 Jun. 2020 | 1.691 | 0.635 | 5.356 | 0.785 | 2.752 | 1.035 | 19.140 | 40.250 |
| 21 | 16 Jun. 2020 | 1.859 | 0.568 | 3.582 | 0.510 | 2.922 | 0.927 | 26.464 | 41.366 |
| 22 | 17 Jun. 2020 | 2.252 | 0.828 | 4.200 | 0.519 | 2.719 | 1.064 | 23.528 | 45.403 |
| 23 | 18 Jun. 2020 | 2.043 | 0.696 | 3.476 | 0.406 | 2.682 | 0.953 | 26.262 | 43.622 |
| 24 | 19 Jun. 2020 | 1.104 | 0.818 | 5.792 | 0.482 | 3.156 | 1.158 | 12.646 | 41.908 |

### Example Study 3

### Background:

Dog dermatologic diseases are frequently associated with pruritus that can lead to skin trauma, skin injury and impaired rest profiles for both owner and dog. Market surveys of dog owners show that the rest profile of their dog is an important concern. Flea and tick infestations are frequent causes of dermatologic disease and can also induce pruritus. Management of canine pruritus is an ongoing requirement with potential for recurrence or ‘flare up’ and with the need to manage anti-pruritic therapy to deliver an effective dose regime.

ANIMO® (provided as a non-limiting example) is a 3-dimensional accelerometer device that detects changes in dog motion (behavior) and enables tracking of such over time. This study illustrates the use of ANIMO's outputs to provide a valuable health profile for veterinarians to document dog therapeutic progress and for dog owners to see the improvement in their dogs' movement profiles with treatment of dermatologic diseases. Dogs wearing ANIMO had an initial 14 days of calibration installation to normalize to the expected movement profile of the individual dog before initiating monitoring.

Dogs in this study were also prescribed BRAVECTO® or another flea/tick medication by their veterinarians as treatment for pruritus from ectoparasites or for skin allergy triggered by ectoparasites. The Florida USA location of the study clinic is an endemic and high-risk region for fleas. Flea/tick medications were prescribed before enrolment in the study and were selected at the discretion of a veterinarian. BRAVECTO® Chews (by Merck Animal Health) are labeled to provide up to 12 weeks of flea and tick protection from a single dose. A key benefit of this extended protection is that dog owners increase their adherence to veterinary recommendations for year-round flea and tick protection by receiving 12 consecutive weeks of therapy without monthly re-dosing. This increased compliance is expected to deliver health benefits for Bravecto-treated dogs when compared to dogs treated with monthly flea/tick products or not treated with flea/tick products.

Dogs enrolled in this field trial wore ANIMO and were seen by a veterinary dermatologist for any cause of pruritus. All of the dogs' behaviors were tracked over the 4-month period following enrolment.

As described further below, this exemplary study illustrates the ability to analyze ANIMO delivered data showing detectable motion (including sleeping, shaking, grooming and scratching) in dogs receiving treatment for pruritus and/or flea infestation and derive useful insight therefrom. As one example, this study illustrates the use of ANIMO delivered data reports as early warning indications of pruritus recurrence (e.g.

‘flare up’) in dogs with allergic conditions. As another example, this study illustrates the ability to evaluate comparative rest profiles for dogs with confirmed dermatologic allergic disease using ANIMO activity data profiles over several months following treatment of pruritus. As another example, this study illustrates the ability to evaluate owner self- reported compliance with recommended flea and tick treatments and the possible role of owner compliance in the comfort of the dog.

### Summary of the Study:

36 dogs were enrolled in the Bravecto group and 1 dog discontinued during the trial. 60 dogs were enrolled in the other treatment group and 7 discontinued during the trial. The total enrollment was 96 dogs with 9 discontinuing so that 87 dogs including 36 (41%) BRAVECTO and 51 (59%) other treatment completed the trial.

Owners maintained notes of medications administered to their dog and other relevant behaviors over the study period as diary notes inserted into the notes field in the app. This is one example of increased communication between pet owners and caregivers.

Owners were asked to sync the ANIMO read-out daily, and this happened automatically as long as ANIMO and app were in bluetooth range and phone had an internet connection. However, there were reports of owners failing to synchronize for more than 72 hours. These were monitored weekly and owners were contacted to get them to synchronize. Any owner who did not synchronize after more than 14 days risked data loss and there were occasional episodes of data loss throughout the trial. A few enrolled dogs were eliminated from the trial for failure to synchronize data. All owners completing the trial were asked to return for a final examination and to complete an end-of-study survey form.

Alerts received during the trial were monitored and each owner contacted. In this example study, alerts indicated either scratching or shaking or possibly both.

### Scratching Analysis by Dermatologic Diagnosis

Several dogs with multiple alerts and/or recurrence of clinical signs as observed by the owner were presented to the veterinarian for re-examination. On average, dogs with atopic dermatitis scratched a similar amount (around 6 min per day) as dogs with allergic dermatitis, as detected by the ANIMO monitor. The “allergy suspected” dogs scratched 2-5 minutes more per day than the other dogs which was consistent month after month. When the scratching time was summed for each dog for each month, atopic dogs scratched 20-30 minutes more per month than allergic dermatitis dogs although this difference was clinically minimal when considered on a daily basis.

Table 14 below shows the mean time (in minutes) of scratching per day dermatologic diagnosis:

| | Otitis | Allergic | Atopic | Allergy |
|---|---|---|---|---|
| | Externa | Dermatitis | Dermatitis | Suspected |
| Mean minutes | 5.8 min | 6.0 min | 6.8 min | 9.1 min |
| per day | | | | |

Attention is also drawn in this respect to FIGS. 12 and 13, which show a graph illustrating the scratching minutes averages per month by dermatologic diagnosis as observed in the study and a graph illustrating the average scratching minutes per day by dermatologic diagnosis as observed in the study, respectively.

### Shaking Analysis by Dermatologic Diagnosis

For the analysis of shaking data, atopic and allergic dermatitis dogs were shaking for an average of 3-4 minutes per day. When shaking minutes were summed for each dog over each month, dogs with allergic dermatitis had approximately 20-30 minutes more shaking per month compared with dogs diagnosed with atopic dermatitis which was less than 1 minute per day.

Table 15 below shows the mean time (in minutes) of shaking per day by dermatologic diagnosis:

| | Otitis | Allergic | Atopic | Allergy |
|---|---|---|---|---|
| | Externa | Dermatitis | Dermatitis | Suspected |
| Mean minutes | 4.4 min | 4.3 min | 3.5 min | 3.6 min |
| per day | | | | |

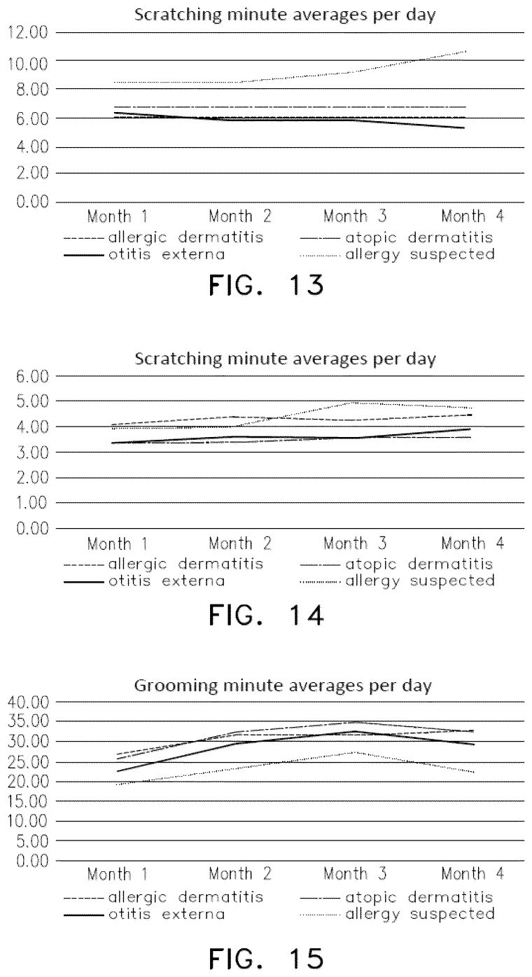

Attention is also drawn in this respect to FIG. 14, which show a graph illustrating the average shaking minutes per day by dermatologic diagnosis as observed in the study.

### Grooming Analysis by Dermatologic Diagnosis

The average grooming minutes per day per dog averaged around 25-35 minutes per day. There did not appear to be much difference in this variable for dogs with atopy compared to allergic dermatitis. Dogs with otitis externa (inflammation limited to the ears) had the lowest amount of daily grooming behaviour detected by ANIMO.

Table 16 below shows the mean time (in minutes) of grooming per day by dermatologic diagnosis:

| | Otitis | Allergic | Atopic | Allergy |
|---|---|---|---|---|
| | Externa | Dermatitis | Dermatitis | Suspected |
| Mean minutes | 22.9 min | 30.5 min | 31.3 min | 28.2 min |
| per day | | | | |

Attention is also drawn in this respect to FIG. 15, which show a graph illustrating the average grooming minutes per day by dermatologic diagnosis as observed in the study.

### Night Rest by Dermatologic Diagnosis

Average minutes of night rest were consistently lowest in dogs with allergic dermatitis and highest in dogs with “allergy suspected”. Night rest averaged 6.9-7.3 hours for dogs diagnosed with atopic dermatitis and allergic dermatitis.

Table 17 below shows the mean time (in hours) of night rest per night by dermatologic diagnosis:

| | Otitis | Allergic | Atopic | Allergy |
|---|---|---|---|---|
| | Externa | Dermatitis | Dermatitis | Suspected |
| Mean hours per night | 7.5 hours | 6.9 hours | 7.3 hours | 8.2 hours |
| % over Allergic | 9% | | 6% | 19% |
| Dermatitis per night | | | | |

Attention is also drawn in this respect to FIG. 16, which show a graph illustrating the average night rest minutes per night by dermatologic diagnosis as observed in the study.

### Sleep Ratio by Dermatologic Diagnosis

The actual vs. reported sleep ratio (actual dog sleep hours per night as determined by Animo data, divided by reported sleep hours reported by the dog owner using App) for the different dermatologic diagnoses were very similar, suggesting that, on average, dogs got similar amounts of quality sleep regardless of the pruritis diagnosis. A sleep ratio of 1 indicates that Animo recorded that the dog slept the entire period which the owner identified as sleep time. A number less than one indicates that the dog was awake for some portion of the sleep time.

Table 18 below shows the mean time (in hours) of night rest per night by dermatologic diagnosis:

| | Otitis | Allergic | Atopic | Allergy |
|---|---|---|---|---|
| | Externa | Dermatitis | Dermatitis | Suspected |
| Average sleep | 0.89 | 0.87 | 0.89 | 0.91 |
| ratio per dog | | | | |

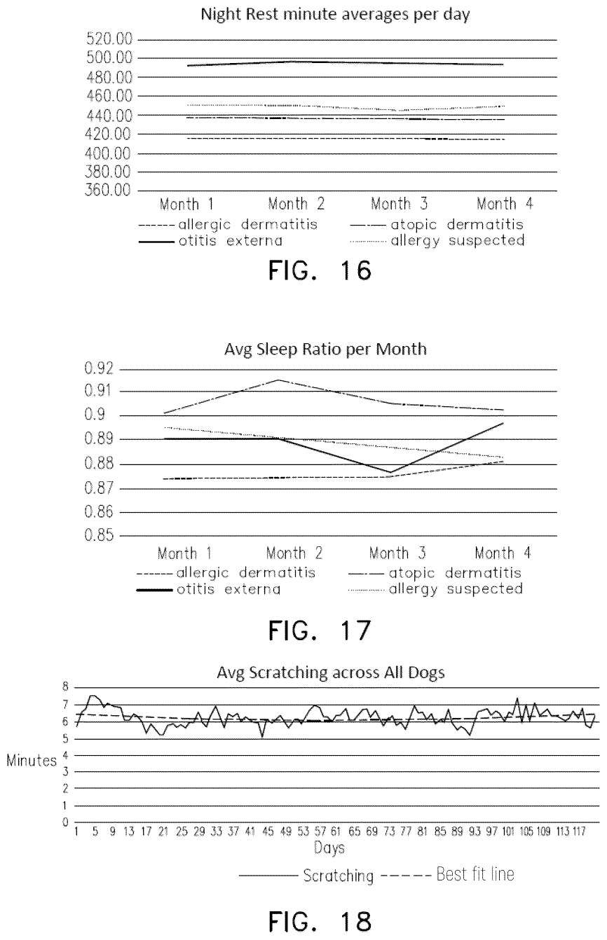

Attention is also drawn in this respect to FIG. 17, which show a graph illustrating the average sleep ratio per night by dermatologic diagnosis as observed in the study.

### Clinical Signs Summary Across All Dogs Over the Entire Study Period

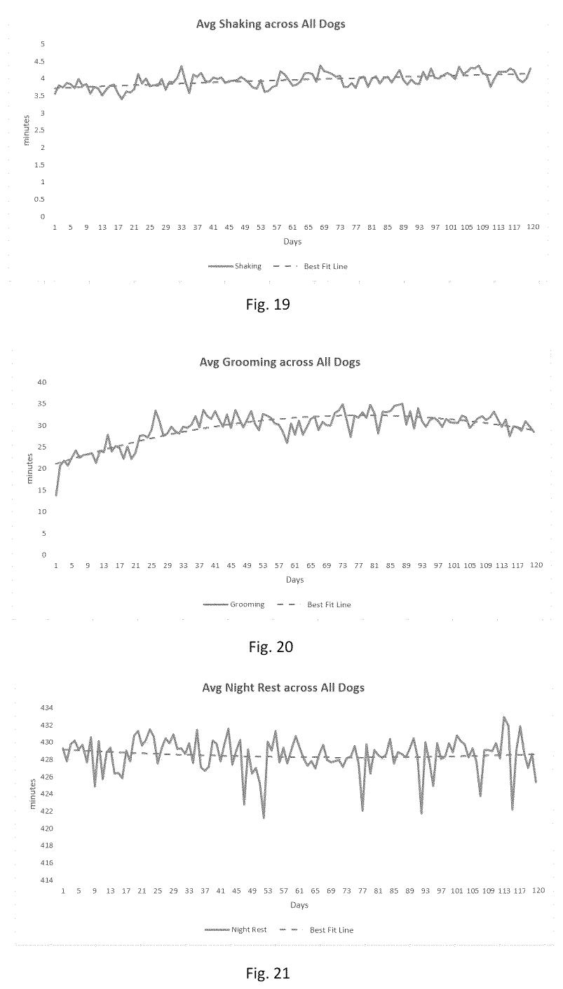

All dogs, regardless of dermatologic diagnoses or flea/tick medication assignment, were combined into a single chart to visualize the pattern of scratching (FIG. 18 shows the average scratching minutes per day during the 120 days study period), shaking (FIG. 19 shows the average shaking minutes per day during the 120 days study period), grooming (FIG. 20 shows the average grooming minutes per day during the 120 days study period) and night rest (FIG. 21 shows the average night rest minutes per night during the 120 days study period), seen over the 120-day study period. As a group, the dogs were consistent in the amount of each behavior exhibited over time. A best fit line was applied to the data to demonstrate this consistency. For shaking and grooming, the best fit line slowly increased over time, although average scratching and night rest did not appear to shift much over time. Dogs were treated for pruritis early in the study when they were seen in the dermatology clinic for pruritis (day 0 and 14). The shift in shaking and grooming correlates with a gradual return of pruritis as time passes following initial treatment.

### Number of Scratching, Shaking and Total Alerts Across 120 Days of Monitoring

There were 77 dogs that completed the full 120 days of study monitoring. These animals were used to compare the number of alerts generated by Animo during the study. Ten dogs were dropped from this analysis because they had less than 120 days in the study, ranging from 91-119 days total. Alerts were triggered by the Animo App interpretation of recorded data indicating a deviation from the expected, baseline, data. The alerts were classified into categories based on algorithms for interpreting recorded data.

### Scratch Alerts

There were 537 scratch alerts generated by 77 dogs over 120 days. Individual dogs in this study generated from 0-29 alerts over 120 days, with the average dog generating 7.4 scratch alerts in total. 17/77 dogs (22%) generated no scratch alerts at all and 60/77 (78%) generated 1 or more alerts.

When the number of scratch alerts were placed into blocked ranges, it was clear that most dogs diagnosed with allergic dermatitis generated very few (0 or 1-5) scratch alerts over the entire study period. Dogs diagnosed with atopic dermatitis were more likely to generate 1-15 scratch alerts over the same period. The difference in the number of scratch alerts generated between allergic dermatitis and atopic dermatitis was statistically significant. The pattern of scratch alerts was similar between dogs assigned to the Bravecto and Monthly flea/tick medication groups. The difference between means for dogs prescribed different flea/tick medications was not statistically significantly different.

Table 19 shows the scratch alerts generated by dermatologic diagnoses:

| | 0 | 1-5 | 6-10 | 11-15 | 16-20 | >21 | Total |
|---|---|---|---|---|---|---|---|
| Allergic | 11 | 16 | 5 | 1 | 4 | 2 | 39 |
| Dermatitis | (28%) | (41%) | (13%) | (3%) | (10%) | (5%) | (100%) |
| Atopic | 4 | 6 | 8 | 10 | 0 | 2 | 30 |
| Dermatitis | (13%) | (20%) | (27%) | (33%) | | (7%) | (100%) |
| Otitis | 2 | 1 | 2 | 1 | 0 | 0 | 6 |
| Externa | (33%) | (17%) | (33%) | (17%) | | | (100%) |
| Allergy | 0 | 1 | 0 | 0 | 1 | 0 | 2 |
| Suspected | | (50%) | | | (50%) | | (100%) |

### Shake Alerts

There were 672 shake alerts generated by 77 dogs over 120 days. Dogs in this study generated from 0-32 alerts over 120 days, with the average dog generating 8.6 shake alerts in total. 7/77 dogs (9%) generated no shake alerts at all and 70/77 (91%) generated 1 or more alerts. The pattern of shake alerts was similar between dogs diagnosed with allergic dermatitis and atopic dermatitis.

Table 20 shows the shake alerts generated by dermatologic diagnoses:

| | 0 | 1-5 | 6-10 | 11-15 | 16-20 | >20 | Total |
|---|---|---|---|---|---|---|---|
| Allergic | 3 | 14 | 10 | 2 | 6 | 4 | 39 |
| Dermatitis | (8%) | (36%) | (26%) | (5%) | (15%) | (10%) | (100%) |
| Atopic | 2 | 9 | 9 | 5 | 4 | 1 | 30 |
| Dermatitis | (7%) | (30%) | (30%) | (17%) | (13%) | (3%) | (100%) |
| Otitis | 1 | 2 | 0 | 1 | 0 | 2 | 6 |
| Externa | (17%) | (33%) | | (17%) | | (33%) | (100%) |
| Allergy | 1 | 1 | 0 | 0 | 0 | 0 | 2 |
| Suspected | (50%) | (50%) | | | | | (100%) |

### Total Alerts

The canine behavior that triggers a scratch alert could be generated independently of the canine behavior that generated a shake alert. In case there was some shared commonality, where one dog doing a particular motion might generate a scratch alert but another dog doing a similar motion might generate a shake alert, we created a category of “total alerts” which was the sum of scratch plus shake alerts.

There were 1209 total alerts generated by 77 dogs over 120 days. Dogs in this study generated from 0-57 total alerts over 120 days, with the average dog generating 16.8 total alerts. 4/77 dogs (5%) generated no total alerts at all, meaning they generated no scratch or shake alerts over the study period. Dogs that generated no alerts came from the allergic dermatitis group (n=2), atopic dermatitis group (n=1) and otitis externa group (n=1). 73/77 (95%) dogs generated 1 or more alerts over the study period. The largest proportion of allergic dermatitis dogs generated 1-10 total alerts with the largest proportion of atopic dermatitis dogs generating 11-20 total alerts, which is a similar pattern seen when we looked at scratch alerts alone.

Table 21 shows the total alerts generated by dermatologic diagnoses:

| | 0 | 1-10 | 11-20 | 21-30 | 31-40 | >40 | Total |
|---|---|---|---|---|---|---|---|
| Allergic | 2 | 16 | 11 | 6 | 3 | 1 | 39 |
| Dermatitis | (5%) | (41%) | (28%) | (15%) | (8%) | (3%) | (100%) |
| Atopic | 1 | 4 | 19 | 3 | 2 | 1 | 30 |
| Dermatitis | (3%) | (13%) | (64%) | (10%) | (7%) | (3%) | (100%) |
| Otitis | 1 | 1 | 2 | 2 | 0 | 0 | 6 |
| Externa | (17%) | (17%) | (33%) | (33%) | | | (100%) |
| Allergy | 0 | 1 | 1 | 0 | 0 | 0 | 2 |
| Suspected | | (50%) | (50%) | | | | (100%) |

On average, allergic dermatitis dogs generated a little over half (63%; 5.6/8.9) of the scratching alerts generated by atopic dermatitis dogs but had a similar average number of shake alerts (9.2 versus 8.3).

Table 22 shows the average number of alerts by dermatologic diagnoses over 120 days:

| | | Scratch | Shake | Total |
|---|---|---|---|---|
| | n | Alerts | Alerts | Alerts |
| Allergic Dermatitis | 39 | 5.6 | 9.2 | 14.7 |
| Atopic Dermatitis | 30 | 8.9 | 8.3 | 17.3 |
| Otitis Externa | 6 | 5.5 | 10.5 | 16.0 |
| Allergy Suspected | 2 | 9.0 | 1.0 | 10.0 |

It is to be understood that the presently disclosed subject matter is not limited in its application to the details set forth in the description contained herein or illustrated in the drawings. The presently disclosed subject matter is capable of other embodiments and of being practiced and carried out in various ways. Hence, it is to be understood that the phraseology and terminology employed herein are for the purpose of description and should not be regarded as limiting. As such, those skilled in the art will appreciate that the conception upon which this disclosure is based may readily be utilized as a basis for designing other structures, methods, and systems for carrying out the several purposes of the present presently disclosed subject matter.

It will also be understood that the system according to the presently disclosed subject matter can be implemented, at least partly, as a suitably programmed computer. Likewise, the presently disclosed subject matter contemplates a computer program being readable by a computer for executing the disclosed methods. The presently disclosed subject matter further contemplates a machine-readable memory tangibly embodying a program of instructions executable by the machine for executing the disclosed methods.

1. A system for identifying irregularities in behaviors of a non-human animal, the system comprising a processing circuitry configured to:
provide a behavioral baseline including first information on regular behaviors of the non-human animal over a given period of time when no irregularities occur;
obtain data on a series of consecutively identified behaviors of the non-human animal identified over a second period of time;
perform an action upon the data not complying with the behavioral baseline, thereby indicating an irregularity in the non-human animal behavior.

2. The system of claim 1, wherein the information on regular behaviors includes, for each regular behavior: (a) an indication of a type of behavior, and (b) one or more of: (i) regular frequency range of the behavior, (ii) regular duration range for the behavior, (iii) regular intensity range for the behavior, (iv) regular score range of a score calculated for the behavior.

3. The system of claim 1, wherein the processing circuitry is further configured to analyze the data to determine a cause for the irregularity.

4. The system of claim 3, wherein the cause is one or more of: pruritus, a cardiological problem, a neurological problem, obesity, diabetes, separation anxiety, arthritis, ear inflammation, a musculo-skeletal problems.

5. The system of claim 1, wherein the consecutively identified behaviors are determined based on analysis of three-dimensional (3D) accelerometer data acquired by a 3D accelerometer comprised in a device attached to the non-human animal

6. The system of claim 1, wherein the behavioral baseline is an animal specific behavioral baseline determined using baseline creation data including a baseline series of consecutively identified baseline behaviors of the non-human animal identified over a third period of time in which the non-human animal is assumed to behave regularly.

7. The system of claim 1, wherein the action is triggering an alert to a caregiver of the non-human animal.

8. The system of claim 1, wherein the regular behaviors and the consecutively identified behaviors include one or more of: shaking, grooming, scratching, resting, sleeping, high-activity, medium activity, low-activity, barking, calories burned, walking, running, sitting, lying, jumping, chewing, sniffing, or licking.

9. The system of claim 1, wherein the information on the regular behaviors includes a sleep score.

10. The system of claim 1, wherein the processing circuitry is further configured to provide one or more irregularity preventing recommendations to a caregiver of the non-human animal based on historical behavioral data associated with the non-human animal.

11. A method for identifying irregularities in behaviors of a non-human animal, the method comprising:
providing, by a processing circuitry, a behavioral baseline including first information on regular behaviors of the non-human animal over a given period of time when no irregularities occur;
obtaining, by the processing circuitry, data on a series of consecutively identified behaviors of the non-human animal identified over a second period of time;
performing, by the processing circuitry, an action upon the data not complying with the behavioral baseline, thereby indicating an irregularity in the non-human animal behavior.

12. The method of claim 11, wherein the information on regular behaviors includes, for each regular behavior: (a) an indication of a type of behavior, and (b) one or more of: (i) regular frequency range of the behavior, (ii) regular duration range for the behavior, (iii) regular intensity range for the behavior, (iv) regular score range of a score calculated for the behavior.

13. The method of claim 12, further comprising analyzing, by the processing circuitry, the data to determine a cause for the irregularity.

14. The method of claim 13, wherein the cause is one or more of: pruritus, a cardiological problem, a neurological problem, obesity, diabetes, separation anxiety, arthritis, ear inflammation, a musculo-skeletal problems.

15. The method of claim 11, wherein the consecutively identified behaviors are determined based on analysis of three-dimensional (3D) accelerometer data acquired by a 3D accelerometer comprised in a device attached to the non-human animal.

16. The method of claim 11, wherein the behavioral baseline is an animal specific behavioral baseline determined using baseline creation data including a baseline series of consecutively identified baseline behaviors of the non-human animal identified over a third period of time in which the non-human animal is assumed to behave regularly.

17. The method of claim 11, wherein the regular behaviors and the consecutively identified behaviors include one or more of: shaking, grooming, scratching, resting, sleeping, high-activity, medium activity, low-activity, barking, calories burned, walking, running, sitting, lying, jumping, chewing, sniffing, or licking.

18. The method of claim 11, wherein the information on the regular behaviors includes a sleep score.

19. The method of claim 11, further comprising providing, by the processing circuitry, one or more irregularity preventing recommendations to a caregiver of the non-human animal based on historical behavioral data associated with the non-human animal

20. A non-transitory computer readable storage medium having computer readable program code embodied therewith, the computer readable program code, executable by at least one processing circuitry of a computer to perform a method for identifying irregularities in behaviors of a non-human animal, the method comprising:
providing, by the processing circuitry, a behavioral baseline including first information on regular behaviors of the non-human animal over a given period of time when no irregularities occur;
obtaining, by the processing circuitry, data on a series of consecutively identified behaviors of the non-human animal identified over a second period of time;
performing, by the processing circuitry, an action upon the data not complying with the behavioral baseline, thereby indicating an irregularity in the non-human animal behavior.
